(19) United States
(12) Patent Application Publication
(10) Pub. No.: US 2005/0055742 A1
(43) Pub. Date: Mar. 10, 2005

(54) Plant regulatory element

(76) Inventors: Brian L. Miki, Ottawa (CA); Jiro Hattori, Ottawa (CA); Teresa Martin-Heller, Gloucester (CA); Helene Labbe, Ottawa (CA); Kamal Malik, West Lafayette, IN (US); Elizabeth Foster, Nepean (CA); Keqiang Wu, Morgantown, WV (US); Daniel Charles William Brown, Ilderton (CA); Lining Tian, London (CA); Therese Ouellet, Nepean (CA); Peijun Zhang, Ottawa (CA); Elizabeth E. James, Arnprior (CA); Pierre Fobert, Saskatoon (CA); Venkatram Iyer, Kelowna (CA)

Correspondence Address: Kathy Smith Dias, Esq.;Heslin Rothenberg Farley & Mesiti P.C., 5 Columbia Circle, Albany, NY, 12203, US

(21) Appl. No.: 10/866,529

(22) Filed: Jun. 10, 2004

Related U.S. Application Data
(63) Continuation-in-part of application No. 09/457,123, filed on Dec. 7, 1999, now abandoned.
Application No. 09/457,123 is a continuation-in-part of application No. 09/174,999, filed on Oct. 19, 1998, now abandoned.
Application No. 09/174,999 is a continuation of application No. 08/593,121, filed on Feb. 1, 1996, now Pat. No. 5,824,872.

(30) Foreign Application Priority Data
Jun. 22, 1999 (WO) PCT/CA99/00578

(51) Int. Cl. C12Q 1/68; C07H 21/04; A01H 1/00; C12N 15/82; C12N 5/04

(52) U.S. Cl. 800/279; 800/294; 530/370; 435/6; 435/419; 536/23.6

(57) ABSTRACT

An nucleotide sequence and that exhibits regulatory element activity is disclosed. The nucleotide sequence may be defined by SEQ ID NO:22, a nucleotide sequence that hybridizes to the nucleic acid sequence of SEQ ID NO:22, or a compliment thereof. Also disclosed is a chimeric construct comprising the nucleotide sequence operatively linked with a coding region of interest. A method of expressing a coding region of interest within a plant by introducing the chimeric construct described above, into the plant, and expressing the coding region of interest is also provided. Also disclosed are plants, seed, or plant cells comprising the chimeric construct as defined above.

This application is a continuation-in-part of U.S. Ser. No. 09/457,123, filed Dec. 7, 1999, which is a continuation-in-part of U.S. Ser. No. 09/174,999, filed Oct. 19, 1998, now abandoned, which is a continuation of U.S. Ser. No. 08/593,121, filed Feb. 1, 1996, now U.S. Pat. No. 5,824,872, issued Oct. 20, 1998, the entire disclosures of each of which are incorporated herein by reference.

### FIELD OF INVENTION

The present invention relates to regulatory elements obtained from a plant. This invention further relates to the use of one or more than one regulatory element to control the expression of exogenous DNAs of interest in a desired host.

### BACKGROUND OF THE INVENTION

Bacteria from the genus Agrobacterium have the ability to transfer specific segments of DNA (T-DNA) to plant cells, where they stably integrate into the nuclear chromosomes. Analyses of plants harbouring the T-DNA have revealed that this genetic element may be integrated at numerous locations, and can occasionally be found within genes. One strategy which has been exploited to identify integration events within genes is to transform plant cells with specially designed T-DNA vectors which contain a reporter gene, devoid of cis-acting transcriptional and translational expression signals (i.e. promoterless), located at the end of the T-DNA. Upon integration, the initiation codon of the promoterless gene (reporter gene) will be juxtaposed to plant sequences. The consequence of T-DNA insertion adjacent to, and downstream of, gene promoter elements may be the activation of reporter gene expression. The resulting hybrid genes, referred to as T-DNA-mediated gene fusions, consist of unknown and thus un-characterized plant promoters residing at their natural location within the chromosome, and the coding sequence of a marker gene located on the inserted T-DNA (Fobert et al., 1991, Plant Mol. Biol. 17, 837-851).

It has generally been assumed that activation of promoterless or enhancerless marker genes result from T-DNA insertions within or immediately adjacent to genes. The recent isolation of several T-DNA insertional mutants (Koncz et al., 1992, Plant Mol. Biol. 20, 963-976; reviewed in Feldmann, 1991, Plant J. 1, 71-82; Van Lijsebettens et al., 1991, Plant Sci. 80, 27-37; Walden et al., 1991, Plant J. 1: 281-288; Yanofsky et al., 1990, Nature 346, 35-39), shows that this is the case for at least some insertions. However, other possibilities exist. One of these possibilities is that integration of the T-DNA activates silent regulatory sequences that are not associated with genes. Lindsey et al. (1993, Transgenic Res. 2, 33-47) referred to such sequences as “pseudo-promoters” and suggested that they may be responsible for activating marker genes in some transgenic lines. Fobert et al. (1994, Plant J. 6, 567-577) have cloned such sequences and have referred to these as “cryptic promoters”.

Mandel et al (1995, Plant Molec. Biol. 29:995-1004) discloses a promoter which is active in leaves, stem, and apical meristem tissues. This promoter was obtained from translation initiation factor 4A (NeIF-4A), a house keeping gene found in metabolically active cells.

Other regulatory elements are located within the 5′ and 3′ untranslated regions (UTR) of genes. These regulatory elements can modulate gene expression in plants through a number of mechanisms including translation, transcription and RNA stability. For example, some regulatory elements are known to enhance the translational efficiency of mRNA, resulting in an increased accumulation of recombinant protein by many folds. Some of those regulatory elements contain translational enhancer sequences or structures, such as the Omega sequence of the 5′ leader of the tobacco mosaic virus (Gallie and Walbot, 1992, Nucleic Acid res. 20, 4631-4638), the 5′ alpha-beta leader of the potato virus X (Tomashevskaya et al, 1993, J. Gen. Virol. 74, 2717-2724), and the 5′ leader of the photosystem I gene psaDb of Nicotiana sylvestris (Yamamoto et al., 1995, J. Biol. Chem 270, 12466-12470). Other 5′ regulatory elements affect gene expression by quantitative enhancement of transcription, as with the UTR of the thylakoid protein genes PsaF, PetH and PetE from pea (Bolle et al., 199, Plant J. 6, 513-523), or by repression of transcription, as for the 5′ UTR of the pollen-specific LA T59 gene from tomato (Curie and McCormick, 1997, Plant Cell 9, 2025-2036). Some 3′ regulatory regions contain sequences that act as mRNA instability determinants, such as the DST element in the Small Auxin-Up RNA (SAUR) genes of soybean and Arabidopisis (Newman et al., 1993, Plant Cell 5, 701-714). Other translational enhancers are also well documented in the literature (e.g. Helliwell and Gray 1995, Plant Mol. Bio. vol 29, pp. 621-626; Dickey L. F. al. 1998, Plant Cell vol 10, 475-484; Dunker B. P. et al. 1997 Mol. Gen. Genet. vol 254, pp. 291-296).

### SUMMARY OF THE INVENTION

The present invention relates to regulatory elements obtained from a plant. This invention further relates to the use of one or more than one regulatory element to control the expression of exogenous DNAs of interest in a desired host.

It is an object of the invention to provide an improved constitutive regulatory element.

The transgenic tobacco plant, T1275, contained a 4.38 kb EcoRI/XbaI fragment containing the 2.15 kb promoterless GUS-nos gene and 2.23 kb of 5′ flanking tobacco DNA (2225 bp). This 5′ flanking DNA shows no homology to known sequences, and exhibits constitutive regulatory element activity. Analysis of the 5′ flanking DNA revealed the occurrence of several additional regulatory elements, and that this DNA is a member of a large family of repetitive elements.

The present invention relates in part to an isolated plant constitutive regulatory element that directs expression in at least ovary, flower, immature embryo, mature embryo, seed, stem, leaf, root and cultured tissues of a plant. preferably, the regulatory element is not obtained from a IFA-4A gene. The isolated plant constitutive regulatory element may also be characterised by lacking an intron in its 5′ UTR and a TATA box.

The constitutive regulatory element could not be detected in soybean, potato, sunflower, Arabidopsis, B. napus, B. oleracea, corn, wheat or black spruce by Southern blot analysis. However, expression of a coding region of interest, under control of the regulatory element, or a fragment thereof, was observed in transgenic tobacco, N. tabacum c.v. Petit Havana, SRI, transgenic B. napus c.v. Westar, transgenic alfalfa, and transgenic Arabidopsis, and was observed in leaf, stem, root, developing seed and flower. In transient expression analysis, GUS activity was also observed in leaf tissue of soybean, alfalfa, Arabidopsis, tobacco, B. napus, pea, potato, peach, Ginseng and suspension cultured cells of white spruce, oat, corn, wheat and barley.

Thus this invention also provides for a regulatory element that is a constitutive regulatory element. Furthermore, this regulatory element functions in diverse plant species when introduced on a cloning vector, and maybe used to drive the expression of a coding region of interest within a range of plant species.

The present invention also relates to an isolated plant regulatory element that directs expression in at least ovary, flower, immature embryo, mature embryo, seed, stem, leaf, root and cultured tissues of a plant, wherein the regulatory element, or a fragment thereof, is a repetitive element. Preferably, the isolated plant regulatory element is a member of the RENT family of repetitive elements.

This invention pertains to a regulatory element characterized in that it comprises at least an 18 bp contiguous sequence of any one of SEQ ID NO's: 1, 5, 6, 7, 8, 9, 21 and 22.

The present invention also embraces a regulatory element having a nucleotide sequence that hybridizes to a nucleotide sequence, or a fragment thereof, as defined by the nucleotide sequence of any one of SEQ ID NO: 1, 5, 6, 7, 8, 9, 21 and 22 under the following hybridization conditions: 4×SSC at 65° C. overnight, followed by washing in 0.1×SSC at 65° C. for one hour, or twice for 30 minutes each, wherin the nucleotide sequence exhibits regulatory element activity.

The transcription start site for the introduced GUS gene in transgenic tobacco was located in the plant DNA upstream of the insertion site. It was the same in leaf, stem, root, seeds and flower. Furthermore, the native site was silent in both untransformed and transgenic tobacco.

This invention also relates to a chimeric construct comprising a coding region of interest for which constitutive expression is desired, and a constitutive regulatory element, comprising at least an 18 bp contiguous sequence of any one SEQ ID NO's: 1, 5, 6, 7, 8, 9, 21 and 22. This invention further relates to a cloning vector containing the chimeric gene construct.

This invention also includes a plant cell which has been transformed with the chimeric gene, or cloning vector as defined above. Furthermore, this invention embraces transgenic plants, and seeds, containing the chimeric gene, or the cloning vector as defined above.

This invention further relates to any transgenic host, for example, but not limited to a transgenic plant, containing a nucleotide sequence selected from the group consisting of SEQ ID NO's: 1, 5, 6, 7, 8, 9, 21 and 22 or nucleic acid sequence that hybridizes to the nucleotide sequence, a complement, or a fragment thereof, as defined by the nucleotide sequence of any one of SEQ ID NO's: 1, 5, 6, 7, 8, 9, 21 and 22 under the following hybridization conditions: 4×SSC at 65° C. overnignt, followed by washing in 0.1×SSC at 65° C. for one hour, or twice for 30 minutes each. The nucleotide sequence may also be operatively linked to a coding region of interest that is transcribed into RNA. Preferably, the coding region is heterologous with respect to the regulatory region.

Also included in the present invention is a method of conferring expression of a coding region of interest in a plant, comprising: operatively linking an exogenous coding region of interest, for which constitutive expression is desired, with a regulatory element comprising at least an 18 bp contiguous sequence of any one of SEQ ID NO's:1, 5, 6, 7, 8, 9, 21 and 22 to produce a chimeric construct and introducing the chimeric construct into a plant, and expressing the coding region of interest.

The present invention also provides an isolated nucleotide sequence comprising the nucleic acid sequence defined by SEQ ID NO:22, a nucleotide sequence that hybridizes to the nucleic acid sequence of SEQ ID NO:22, or a nucleotide sequence that hybridizes to a compliment of the nucleotide sequence of SEQ ID NO:22, wherein hybridization condition is selected from the group consisting of
hybridizing overnight in a solution comprising 7% SDS, 0.5M NaPO4 buffer at pH 7.2, and 10 mM EDTA at 65° C. and washing for one hour at 60° C. in a solution comprising 0.1×SSC and 0.1% SDS;
hybridizing overnight in a solution comprising 7% SDS, 0.5M NaPO4 buffer at pH 7.2, and 10 mM EDTA at 65° C. and washing for one hour at 65° C. in a solution comprising 2×SSC and 0.1% SDS; and
hybridizing overnight in a solution comprising 4×SSC at 65° C. and washing one hour in 0.1×SSC at 65° C., and
   wherein the nucleotide sequence exhibits regulatory element activity and is capable of mediating transcriptional efficiency of a transcript encoding a gene of interest.

The present invention also pertains to a chimeric construct comprising the isolated nucleotide sequence as just described operatively linked with a coding region of interest. Furthermore, the present invention provides a method of expressing a coding region of interest within a plant comprising introducing the chimeric construct just defined, into a plant, and expressing the coding region of interest. The invention also includes a plant comprising the chimeric construct, a seed comprising the chimeric construct, a plant cell comprising the chimeric construct. The plant, seed or plant cell may be selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

The present invention pertains to the isolated nucleotide sequence a just defined, wherein the nucleotide sequence is defined by SEQ ID NO: 1, 5, 6, 7,8, 9, 21 Or 22, a nucleic acid sequence that hybridizes to the nucleotide sequence of SEQ ID NO:1, 5, 6, 7, 8, 9, 21 or 22, or a nucleic acid sequence that hybridizes to a compliment of the nucleotide sequence of SEQ ID NO: 1, 5, 6, 7, 8, 9, 21 or 22.

The present invention also pertains to a chimeric construct comprising the isolated nucleotide sequence as just described operatively linked with a coding region of interest. Furthermore, the present invention provides a method of expressing a coding region of interest within a plant comprising introducing the chimeric construct just defined, into a plant, and expressing the coding region of interest. The invention also includes a plant comprising the chimeric construct, a seed comprising the chimeric construct, a plant cell comprising the chimeric construct. The plant, seed or plant cell may be selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

The present invention also provides an isolated nucleotide sequence comprising the nucleic acid sequence defined by nucleotides 1660-1875 of SEQ ID NO: 1, a nucleotide sequence that hybridizes to nucleotides 1660-1875 of SEQ ID NO: 1, or a nucleotide sequence that hybridizes to a compliment of nucleotides 1660-1875 of SEQ ID NO: 1, wherein hybridization condition is 65° C. over night in 7% SDS; 0.5M NaPO₄; 10 mM EDTA, followed by two washes at 50° C. in 0.1×SSC, 0.1% SDS for 30 minutes each, wherein the nucleotide sequence exhibits regulatory element activity and is capable of mediating transcriptional efficiency of a transcript encoding a gene of interest.

The present invention also pertains to a chimeric construct comprising the isolated nucleotide sequence as just described operatively linked with a coding region of interest. Furthermore, the present invention provides a method of expressing a coding region of interest within a plant comprising introducing the chimeric construct just defined, into a plant, and expressing the coding region of interest. The invention also includes a plant comprising the chimeric construct, a seed comprising the chimeric construct, a plant cell comprising the chimeric construct. The plant, seed or plant cell may be selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

The present invention pertains to the isolated nucleotide sequence just defined, wherein the nucleotide sequence is defined by nucleotides 1660-1992 of SEQ ID NO:1.

The present invention also pertains to a chimeric construct comprising the isolated nucleotide sequence as just described operatively linked with a coding region of interest. Furthermore, the present invention provides a method of expressing a coding region of interest within a plant comprising introducing the chimeric construct just defined, into a plant, and expressing the coding region of interest. The invention also includes a plant comprising the chimeric construct, a seed comprising the chimeric construct, a plant cell comprising the chimeric construct. The plant, seed or plant cell may be selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

The present invention relates to an isolated nucleotide sequence comprising the nucleic acid sequence defined by nucleotides 2091-2170 of SEQ ID NO: 1, a nucleotide sequence that hybridizes to nucleotides 2091-2170 of SEQ ID NO: 1, or a nucleotide sequence that hybridizes to a compliment of nucleotides 2091-2170 of SEQ ID NO: 1, wherein hybridization condition is 65° C. over night in 7% SDS; 0.5M NaPO₄; 10 mM EDTA, followed by two washes at 50° C. in 0.1×SSC, 0.1% SDS for 30 minutes each, wherein the nucleotide sequence exhibits regulatory element activity and is capable of mediating transcriptional efficiency of a transcript encoding a gene of interest.

The present invention also pertains to a chimeric construct comprising the isolated nucleotide sequence as just described operatively linked with a coding region of interest. Furthermore, the present invention provides a method of expressing a coding region of interest within a plant comprising introducing the chimeric construct just defined, into a plant, and expressing the coding region of interest. The invention also includes a plant comprising the chimeric construct, a seed comprising the chimeric construct, a plant cell comprising the chimeric construct. The plant, seed or plant cell may be selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

The present invention also pertains to the isolated nucleotide sequence as just described, wherein the nucleotide sequence is defined by nucleotides 1660-2224 of SEQ ID NO: 1, 1723-2224 of SEQ ID NO: 1, 415-2224 of SEQ ID NO: 1, 1040-2224 of SEQ ID NO:1, 1370-2224 of SEQ ID NO:1, 2084-2224 of SEQ ID NO:1, or 2042-2224 of SEQ ID NO: 1.

The present invention also pertains to a chimeric construct comprising the isolated nucleotide sequence as just described operatively linked with a coding region of interest. Furthermore, the present invention provides a method of expressing a coding region of interest within a plant comprising introducing the chimeric construct just defined, into a plant, and expressing the coding region of interest. The invention also includes a plant comprising the chimeric construct, a seed comprising the chimeric construct, a plant cell comprising the chimeric construct. The plant, seed or plant cell may be selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

The present invention provides an isolated nucleotide sequence comprising the nucleic acid sequence defined by nucleotides 1875-1992 of SEQ ID NO: 1, a nucleotide sequence that hybridizes to nucleotides 1875-1992 of SEQ ID NO: 1, or a nucleotide sequence that hybridizes to a compliment of nucleotides 1875-1992 of SEQ ID NO: 1, wherein hybridization condition is 65° C. over night in 7% SDS; 0.5M NaPO₄; 10 mM EDTA, followed by two washes at 50° C. in 0.1×SSC, 0.1% SDS for 30 minutes each, wherein the nucleotide sequence exhibits regulatory element activity and is capable of mediating transcriptional efficiency of a transcript encoding a gene of interest.

The present invention also pertains to a chimeric construct comprising the isolated nucleotide sequence as just described operatively linked with a coding region of interest. Furthermore, the present invention provides a method of expressing a coding region of interest within a plant comprising introducing the chimeric construct just defined, into a plant, and expressing the coding region of interest. The invention also includes a plant comprising the chimeric construct, a seed comprising the chimeric construct, a plant cell comprising the chimeric construct. The plant, seed or plant cell may be selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

The present invention pertains to an isolated nucleotide sequence as just described, wherein the nucleotide sequence is defined by nucleotides 1875-2084 of SEQ ID NO: 1. Furthermore, the nucleotide sequence defined by nucleotides 1875-2084 of SEQ ID NO: 1 may be present in tandem.

The present invention also pertains to a chimeric construct comprising the isolated nucleotide sequence as just described operatively linked with a coding region of interest. Furthermore, the present invention provides a method of expressing a coding region of interest within a plant comprising introducing the chimeric construct just defined, into a plant, and expressing the coding region of interest. The invention also includes a plant comprising the chimeric construct, a seed comprising the chimeric construct, a plant cell comprising the chimeric construct. The plant, seed or plant cell may be selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

The present invention also provides an isolated nucleotide sequence comprising the nucleic acid sequence defined by nucleotides 1-1660 of SEQ ID NO: 1, a nucleotide sequence that hybridizes to nucleotides 1875-1660 of SEQ ID NO: 1, or a nucleotide sequence that hybridizes to a compliment of nucleotides 1-1660 of SEQ ID NO: 1, wherein hybridization condition is 65° C. over night in 7% SDS; 0.5M NaPO₄; 10 mM EDTA, followed by two washes at 50° C. in 0.1×SSC, 0.1% SDS for 30 minutes each, wherein the nucleotide sequence exhibits regulatory element activity and is capable of mediating transcriptional efficiency of a transcript encoding a gene of interest.

The present invention also pertains to a chimeric construct comprising the isolated nucleotide sequence as just described operatively linked with a coding region of interest. Furthermore, the present invention provides a method of expressing a coding region of interest within a plant comprising introducing the chimeric construct just defined, into a plant, and expressing the coding region of interest. The invention also includes a plant comprising the chimeric construct, a seed comprising the chimeric construct, a plant cell comprising the chimeric construct. The plant, seed or plant cell may be selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

The present invention provides an isolated nucleotide sequence comprising the following nucleic acid sequence:

| | | | | |
|---|---|---|---|---|
| TTATAATTAC | AAAATTGATT | MTAGTWYYTT | TAATTTAATR | YTTWTACATT |
| ATTAATTAAY | TTAGHWSTTT | YAATTYDTTT | TCARAAAYYA | TTTTACTATK |
| KTT(T/-)RT | AAAAWMAAAR | GGRRAAARTG | GYTATTTAAA | TACYAAC(M/-) |
| CTATTTYATT | TCAATTWTAR | CCTAAAATCA | R(M/-)CCC(C/-) | ARTTARCCCC |
| (W/-)(A/-) | (T/-)(T/-) | (Y/-)(C/-) | (A/-)(A/-) | (A/-)(T/-) |
| (T/-)(C/-) | AAAYGGBMYA | KCCCARTTCC | TAAA(A/-)Y | RACYCDCYCC |
| TAACCC(K/-) | (C/-)(T/-) | (T/-)(W/-) | (T/-)(C/-) | (C/-)(A/-) |
| (A/-)(C/-) | (C/-)(C/-) | RCCCKRTTYC | CYCTTTTGAT | CCAGGYYGTT |
| GATCATTTTG | ATCAACGVCC | ARAATTTCCC | CYTTYC(Y/-) | (K/-)TTTT |
| TMATTCCCAA | ACACC(S/-) | CCYAAMYYTA | TCCCRTTTCT | CACCAACCGC |
| CAGATMT(R/-) | (W/-)(A/-) | (T/-)CCTCT | TATCTCTCAA | ACTCTCTCGA |
| ACCTTCCCCT | AACCCTAGCA | GCCTCTCATC | ATCCTCACCT | CAAAACCCAC |
| CGGMMWMCAT | GCCYTCTMRA | G(S/-)(M/-) | (K/-)(Y/-) | (G/-)(R/-) |
| (W/-)(M/-) | (M/-)(C/-) | (C/-)(K/-) | (K/-)(R/-) | (T/-)(R/-) |
| (S/-)(T/-) | (C/-)(A/-) | (S/-)(Y/-) | YCCYYD(T/-) | (G/-)(Y/-) |
| (N/-)(M/-) | (T/-)(T/-) | (A/-), | | |

a nucleotide sequence that hybridizes to the nucleic acid sequence, or a nucleotide sequence that hybridizes to a compliment of the nucleotide sequence, where R is G or A; Y is T or C; M is A or C; K is G or T; S is G or C; W is A or T; B is G or C or T; D is A or G or T; H is A or C or T; and N is A or C or T or G, and wherein hybridization is selected from the group consisting of:
hybridizing overnight in a solution comprising 7% SDS, 0.5M NaPO4 buffer at pH 7.2, and 10 mM EDTA at 65° C. and washing for one hour at 60° C. in a solution comprising 0.1×SSC and 0.1% SDS;
hybridizing overnight in a solution comprising 7% SDS, 0.5M NaPO4 buffer at pH 7.2, and 10 mM EDTA at 65° C. and washing for one hour at 65° C. in a solution comprising 2×SSC and 0.1% SDS; and
hybridizing overnight in a solution comprising 4×SSC at 65° C. and washing one hour in 0.1×SSC at 65° C., and
   wherein the nucleotide sequence exhibits regulatory element activity and is capable of mediating transcriptional efficiency of a transcript encoding a gene of interest.

The present invention also pertains to a chimeric construct comprising the isolated nucleotide sequence as just described operatively linked with a coding region of interest. Furthermore, the present invention provides a method of expressing a coding region of interest within a plant comprising introducing the chimeric construct just defined, into a plant, and expressing the coding region of interest. The invention also includes a plant comprising the chimeric construct, a seed comprising the chimeric construct, a plant cell comprising the chimeric construct. The plant, seed or plant cell may be selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

The present invention discloses transgenic plants generated by tagging with a promoterless GUS (β-glucuronidase) T-DNA vector and the isolation and characterization of a regulatory element identified using this protocol. Cloning and characterization of this insertion site uncovered a unique regulatory element not conserved among related species. The novel constitutive regulatory element is expressed in tissues throughout a plant and across a broad range of plant species. The novel constitutive regulatory element as described herein comprises additional regulatory elements, and is a member of a large family of repetitive elements that also exhibit regulatory element activity. Therefore, the present invention also describes one or more than one novel regulatory element and its homologs. Furthermore, novel non-translated 5′ sequences have been identified within the regulatory element that function as post transcriptional regulatory elements.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows the constitutive expression of GUS in all tissues of plant T1275, including leaf segments (a), stem cross-sections (b), roots (c), flower cross-sections (d), ovary cross-sections (e), immature embryos (f), mature embryos (g), and seed cross-sections (h).

FIG. 2 shows GUS specific activity within a variety of tissues throughout the plant T1275, including leaf (L), stem (S), root (R), anther (A), petal (P), ovary (O), sepal (Se), seeds 10 days post anthesis (S 1), and seeds, 20 days post anthesis (S2).

FIG. 3 is the Southern blot analysis of Eco RI digested T1275 DNA with a GUS gene coding region probe (lane 1) and a nptII gene coding region probe (lane 2) revealing a single T-DNA insertion site in plant T1275.

FIG. 4 shows the cloned GUS gene fusion from pT1275. FIG. 4(A) shows a restriction map of the plant DNA sequence fused with GUS. FIG. 4(B) shows the restriction map of the plant DNA. The arrow indicates the GUS mRNA start site within the plant DNA sequence.

FIG. 5 shows deletion constructs of the T1275 (tCUP) regulatory element, and several results obtained with these constructs. FIG. 5(A) shows the restriction map of the plant DNA of pT1275 upstream from the GUS insertion site. FIG. 5(B) shows further deletion constructs of −62-GUS-nos, −12-GUS-nos, −62(-tsr)-GUS-nos and +30-GUS-nos, relative to −197-GUS-nos (see FIG. 5(A)). FIG. 5(C) shows the 5′ endpoints of each construct as indicated by the restriction endonuclease site, relative to the full length T1275 (tCUP) regulatory element, the arrow indicates the transcriptional start site. Plant DNA is indicated by the solid line, the promoterless GUS-nos gene is indicated by the open box and the shaded box indicates the region coding for the amino terminal peptide fused to GUS. The XbaI fragment in pT1275 was subcloned to create pT1275-GUS-nos. Deletion constructs truncated at the SphI, PstI, SspI, BstYI, and DraI sites were also subcloned to create −1639-GUS-nos, −1304-GUS-nos, −684-GUS-nos, −394-GUS-nos, and −197-GUS-nos, respectively. FIG. 5(D) shows modified constructs of the T1275 regulatory elements. T1275 is indicated by the open box, the CaMV35S promoter element is indicated by the black box. The activity of these constructs is also indicated. GUS activity was determined in tobacco leaves following transient expression using microparticle bombardment. TA30-GUS: a TATATAA element was inserted into the −30 position of −62-GUS; TA35S-GUS: the −62 to −20 fragment of −62-GUS was substituted with the −46 to −20 fragment of the 35S promoter; GCC-62-GUS: a GCC box was fused with −62-GUS; DRA2-GUS: the −197 to −62 fragment was repeated; BST2-GUS: the −394 to −62 fragment was repeated; −46-35S: 35S minimal promoter; DRAI-35S: the −197 to −62 fragment of T1275 was fused with −46-35S; BSTI-35S: the −394 to −62 fragment of T1275 was fused with −46-35S; BST2-35S: two copies of the −394 to −62 fragment of T1275 were fused with −46-35S. FIG. 5(E) shows constructs of the −197 to −62 fragment fused with the 35S minimal promoter. −46-35S: 35S minimal promoter; DRAI-35S: the −197 to −62 fragment of T1275 was fused with −46-35S; DRA1R-35S: the −197 to −62 fragment of T1275 was fused with −46-35S in a reversed orientation; DRA2-35S: two copies of the −197 to −62 fragment of T1275 were fused with −46-35S. FIG. 5(F) shows GUS specific activity of transgenic Arabidopsis plants. Leaf tissues from Arabidopsis plants transformed with −47-35S, DRA1-35S, DRA1R-35S and DRA2-35S constructs were used for GUS assay. FIG. 5(G) shows the constitutive expression of GUS in Arabidopsis plants transformed with DRA1-35S. From top to bottom (i.e. FIGS. 5G(i), 5G(ii) and 5G(iii), respectively): flower, silque and seedling. FIG. 5(H) shows the schematic diagram of the chimerical constructs. The numbers on the top indicate deletion end points relative to the transcription initiation site (+1) of the tCUP. The position of transcription start site is indicated by an arrow. The dot line indicates the sequence been deleted. These constructs include (see FIG. 5(B) for more information): “-62” (−62T1275-GUS-nos); “−12” (−12T1275-GUS-nos); “−62-tsr” (−61 (-tsr)-GUS-nos); TA30 (sequence −30 to −24 of T1275 is replaced with TATATAA); GCC-62 (addition of GCC-box sequences). FIG. 5(I) shows the relative activity of the constructs outlined in FIG. 5(H) within tomato protoplasts. Each value represents the average of four independent experiments. Error bars indicate SE values. FIG. 5(J) shows schematic diagrams of the 5′ deletions chimerical constructs. −394(2×)-GUS and −197(2×)-GUS are the two constructs to test the effect of reiteration of the tCUP upstream regions (−394 to −62 and −197 to −62) on promoter activity. The numbers on the top indicate deletion end points relative to the transcription initiation site (+1) of the tCUP promoter. FIG. 5(K), shows the average GUS specific activity (pmol MU/min/mg protein) in transgenic Arabidopsis plants containing constructs shown in FIG. 5(J). 15-20 independent transgenic plants were tested for each construct. FIG. 5(L) shows the schematic diagram of chimerical constructs to study the effect of the tCUP upstream region −197 to −62 on −46 minimal CaMV 35S promoter activity. The numbers on the top indicate deletion end points relative to the transcription initiation site. Open-boxes represent the tCUP sequence and filled-boxes represent the CaMV 35S promoter sequence. FIG. 5(M) shows the verage GUS specific activity (pmol MU/min/mg protein) in transgenic Arabidopsis plants containing constructs shown in A. 15-20 independent transgenic plants were tested for each construct.

FIG. 6 shows the GUS specific activity, mRNA, and protein levels in leaves of individual, regenerated, greenhouse-grown transgenic tobacco plants containing T1275-GUS-nos (T plants), or 35S-GUS-nos (S plants). FIG. 6(A) shows the levels of GUS expression in leaves from randomly selected plants containing either T1275-GUS-nos (left-hand side) or 35S-GUS-nos (right-hand side). FIG. 6(B) shows the level of accumulated GUS mRNA measured by RNase protection assay and densitometry of autoradiograms in leaves from the same randomly selected plants containing either T1275-GUS-nos (left-hand side) or 35S-GUS-nos (right-hand side). FIG. 6(C) shows a Western blot of GUS fusion protein obtained from T1275-GUS-nos and 35S-GUS-nos plants. Leaf extracts were equally loaded onto gels and GUS was detected using anti-GUS antibodies. The molecular weight markers are indicated on the right-hand side of the gel; untransformed control (SRI) and GUS produced in E. coli (Ec).

FIG. 7 shows deletion and insertion constructs of the 5′ untranslated leader region of T1275 regulatory element and construction of transformation vectors. The constructs are presented relative to T1275-GUS-nos or 35S-GUS-nos. The arrow indicates the transcriptional start site. Plant DNA is indicated by the solid line labeled T1275, the 35S regulatory region by the solid line labelled CaMV35S, the NdeI-SmaI region by a filled in box, the shaded box coding for the amino terminal peptide, and the promoterless GUS-nos gene is indicated by an open box. The deletion construct removing the NdeI-SmaI fragment of T1275-GUS-nos is identified as T1275-N-GUS-nos. The NdeI-SmaI fragment from T1275-GUS-nos was also introduced into 35S-GUS-nos to produce 35S+N-Gus-nos.

FIG. 8 shows the region surrounding the insertion site in untransformed plants, positions of various probes used for RNase protection assays, and results of the RNase protection assay. FIG. 8(A) shows a restriction map of the insertion site and various probes used for the assay (IP: insertion point of GUS in transformed plants; *: that T1275 probe ended at the BstYI site, not the IP; **: probe 7 included 600 bp of the T1275 plant sequence and 400 bp of the GUS gene). FIG. 8(B) shows results of an RNase protection assay of RNA isolated from leaf (L), stem (St), root (R), flower bud (F) and developing seed (Se) tissues of tobacco transformed with T1275-GUS-nos (10 μg RNA) and untransformed tobacco (30 μg RNA). Undigested probe (P), tRNA negative control (−) lanes and markers are indicated. RNase protection assays shown used a probe to detect sense transcripts between about −446 and +596 of T1275-GUS-nos or between about −446 to +169 of untransformed tobacco. The protected fragment in transformed plants is about 596 bp (upper arrowhead) and, if present, accumulated transcripts initiated at this site in untransformed plants are predicted to protect a fragment of about 169 bp (lower arrowhead). Upper band in RNA-containing lanes was added to samples to indicate loss of sample during assay.

FIG. 9 shows the levels of mRNA, as well as the ratio between GUS specific activity and mRNA levels in leaves of individual, regenerated, greenhouse-grown transgenic plants containing T1275-GUS-nos (i.e. tCUP-GUS-nos), or 35S-GUS-nos constructs, with or without the NdeI-SmaI fragment (see FIG. 7). FIG. 9(A) shows the level of accumulated GUS mRNA measured by RNase protection assay and densitometry of autoradiograms in leaves from the same randomly selected plants containing either T1275-GUS-nos, T1275-N-GUS-nos. FIG. 9(B) shows the level of accumulated GUS mRNA measured by RNase protection for 35S-GUS-nos or 35S+N-GUS-nos. FIG. 9(C) shows the ratio between GUS specific activity and mRNA levels in leaves of individual, regenerated, greenhouse-grown transgenic plants containing tCUP-GUS-nos, tCUP-N-GUS-nos, 35S-GUS-nos, or 35S+N-GUS-nos constructs.

FIG. 10 shows the maps of T1275-GUS-nos and T1275(ΔN)-GUS-nos. FIG. 10(A) shows T1275-GUS-nos (also referred to as tCUP-GUS-nos). FIG. 10(B) shows T1275(ΔN)-GUS-nos (also referred to as tCUPdelta-GUS-nos). “ΔN”, (also referred to as “dN” or “deltaN”) was created by changing the NdeI site “a” in the leader sequence of T1275-GUS-nos (FIG. 10(A)) to a BglII site “b” (see FIG. 10(B)) to eliminate the upstream ATG at nucleotides 2087-2089 of SEQ ID NO:2. A Kozak consensus sequence “c” was constructed at the initiator MET codon and a NcoI site was added. The transcriptional start site, determined for T1275, is indicated by the arrow.

FIG. 11 shows constructs used for the transient expression via particle bombardment of corn callus. Maps for 35S-GUS-nos, 35S (+N)-GUS-nos, 35S (ΔN)-GUS-nos and 35S(+i)-GUS-nos are presented indicating the “N” region, ADH1 intron, and the arrow indicates the transcriptional start site. Note that 35S(ΔN)-GUS-nos is referred to as 35S+deltaN-dK-GUS-nos. Also shown are the associated activities of the constructs in the callus expressed as a ratio of GUS to luciferase (control) activity.

FIG. 12 shows maps of the constructs used for transient expression in yeast. Shown are pYES-GUS-nos (also referred to as PYEGUS); pYES(+N)-GUS-nos (also referred to as PYENGUS); pYES(AN)-GUS-nos (also referred to as pYEdNGUS) and pYES(ΔN^{M})-GUS-nos (also referred to as pYEdN^{M}GUS), which lacks the Kozak consensus sequence.

FIG. 13 shows the sequence similarity between several members of the RENT family of highly repetitive sequences. FIG. 13(A) shows a homology tree of an approximately 600 bp fragment of RENT 1 (SEQ ID NO:5), RENT 2 (SEQ ID NO:6), RENT 3 (SEQ ID NO:7), RENT 5 (SEQ ID NO:8), RENT 7 (SEQ ID NO:9) and T1275 (tCUP; SEQ ID NO: 1). FIG. 13(B) shows a graphic representation of the sequence alignments between the different RENT clones and T1275 (tCUP). FIG. 13(C) shows the actual sequence alignments of FIG. 13(B), where the numbering above the sequences indicates the numbering relative to RENT 7 (SEQ ID NO:9), and the numbering below the sequences indicate the alignment of the RENT consensus sequence (SEQ ID NO:21) relative to the tCUP sequence (SEQ ID NO: 1). The consensus sequence relative to tCUP is presented. Inserts within the RENT consensus nucleotide sequence that are not present in tCUP, are indicated above the consensus sequence. Deletions in the nucleotide sequence in at least one member of the RENT family of nucleotide sequences that are not present in tCUP, are indicated as “-” above the consensus sequence. R is G or A; Y is T or C; M is A or C; K is G or T; S is G or C; W is A or T; B is G or C or T; D is A or G or T; H is A or C or T; and N is A or C or T or G. FIG. 13(D) shows the RENT consensus sequence (SEQ ID NO:21), see legend of FIG. 13(C) for details of sequence presentation. FIG. 13(E) shows the nucleotide sequence for tCUP-RENT (SEQ ID NO:22) where nucleotides 1-1723 comprise the nucleotide sequence of tCUP (SEQ ID NO: 1), and nucleotides from 1724 to 2224 comprise the RENT consensus sequence (SEQ ID NO:21).

FIG. 14 shows the expression of a coding region of interest driven by regulatory elements obtained from several members of the RENT family of highly repetitive sequences. FIG. 14(A) shows the transient expression of constructs comprising a RENT regulatory element in operative association with GUS-nos, and the expression of these constructs in pea protoplasts. The constructs were introduced into pea protoplasts via electroporation (see methods for details). tCUP RENT (PCR fragment from 1772 of SEQ ID NO: 1 fused to delta N); RENT 1 (SEQ ID NO:5), RENT 2 (SEQ ID NO:6), RENT 3 (SEQ ID NO:7), RENT 5 (SEQ ID NO:8), RENT 7 (SEQ ID NO:9), 35S-46 (35S minimal promoter. FIG. 14(B) shows histochemical analysis of GUS expression in transgenic Arabidopsis plants containing −394tCUP-GUS construct. GUS gene was expressed in leaves, stems, flowers, siliques and roots of transgenic Arabidopsis plants.

### DETAILED DESCRIPTION

The present invention relates to regulatory elements obtained from a plant. This invention further relates to the use of one or more than one regulatory element to control the expression of exogenous DNAs of interest in a desired host.

The following description is of a preferred embodiment.

T-DNA tagging with a promoterless β-glucuronidase (GUS) gene generated several transgenic Nicotiana tabacum plants that expressed GUS activity. An example, which is not to be considered limiting in any manner, of transgenic plants displaying expression of the promoterless reporter gene, includes a plant that expressed GUS in all organs, T1275 (see co-pending patent applications U.S. Ser. No. 08/593,121, PCT/CA97/00064, and PCT/CA99/0057 which are incorporated by reference).

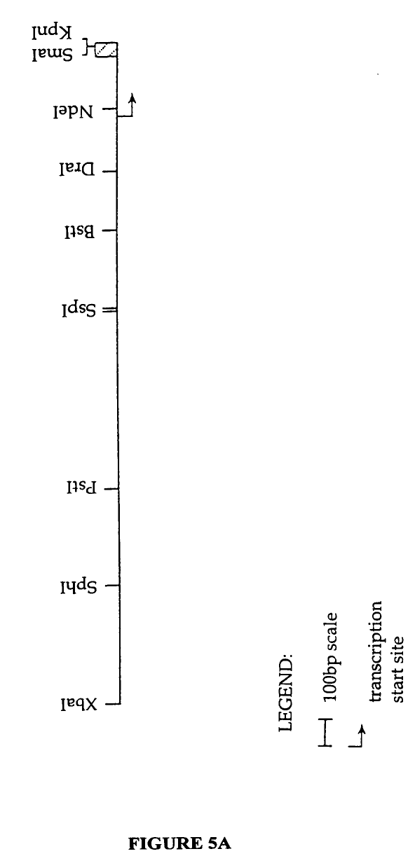

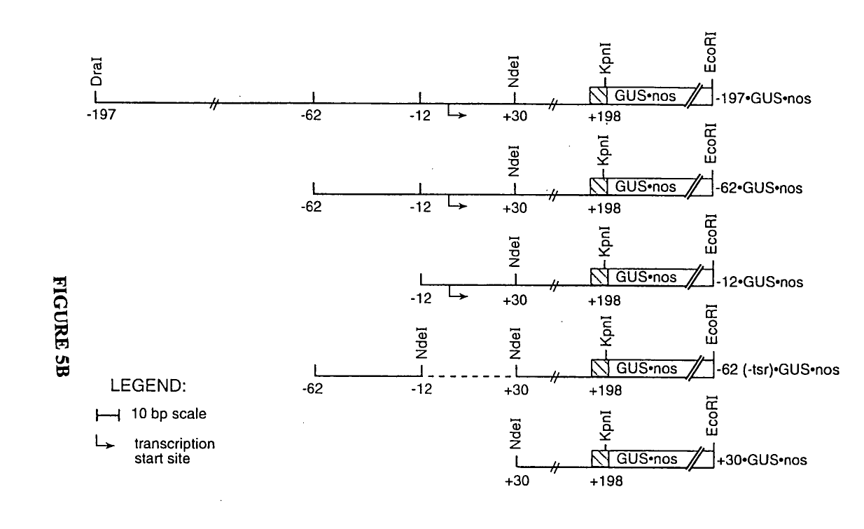

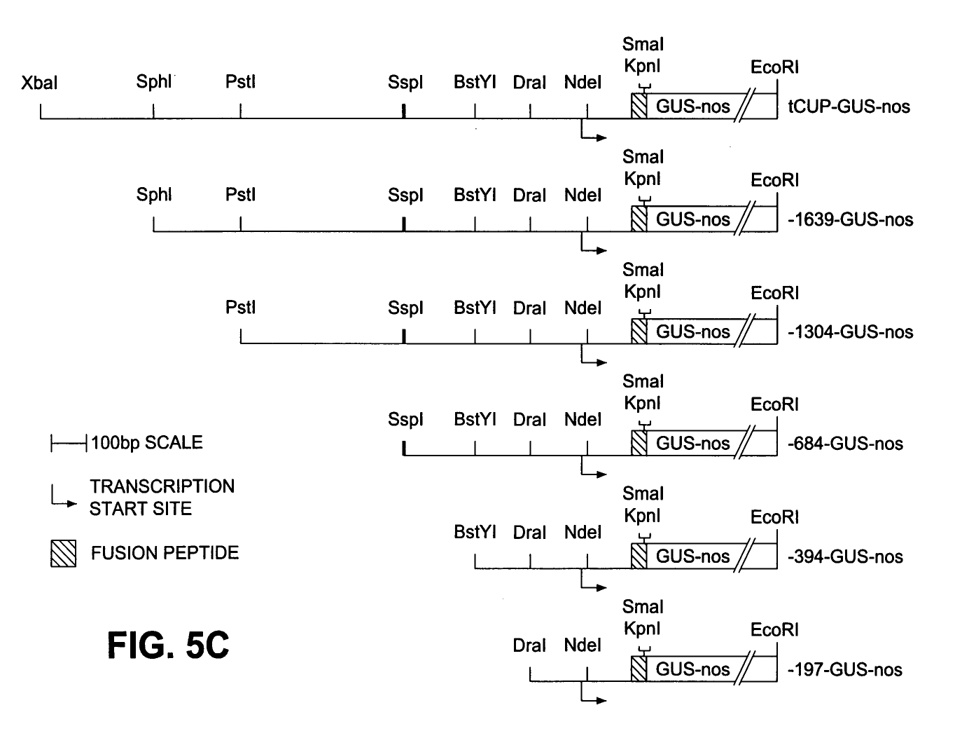

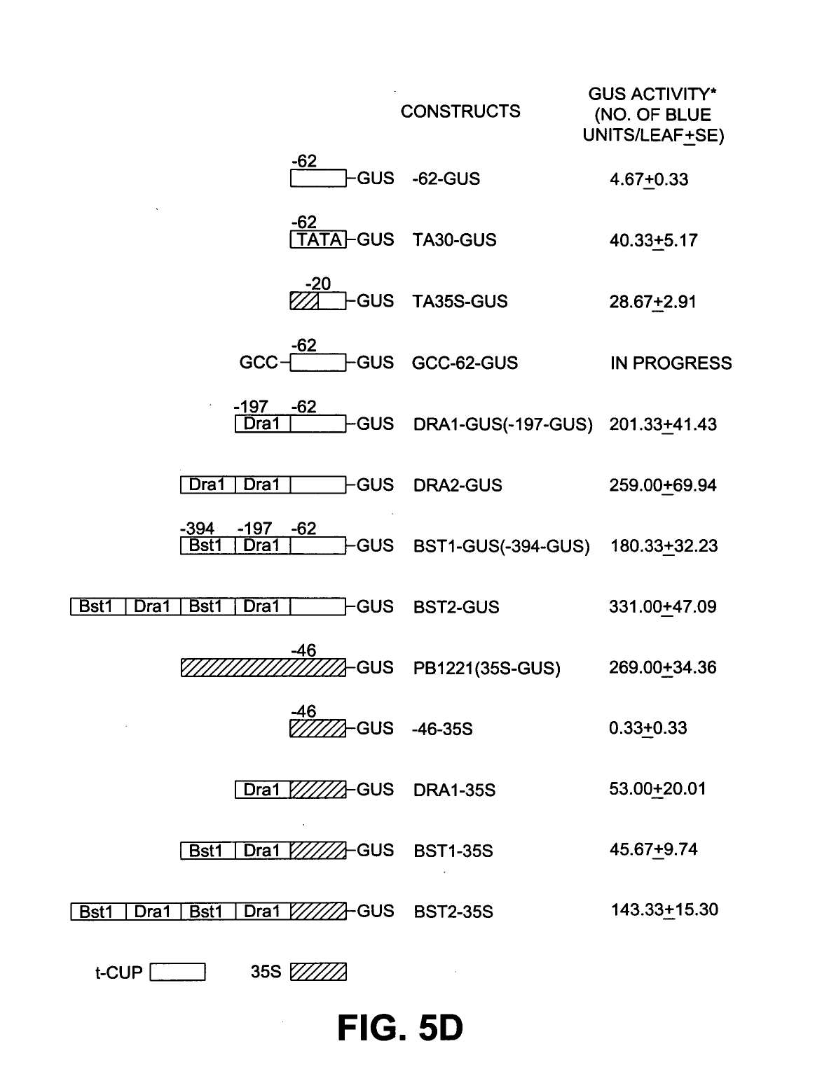

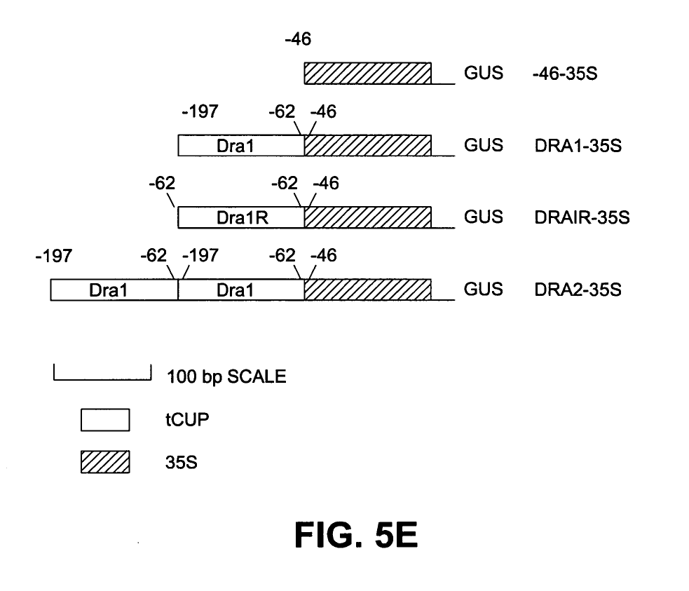

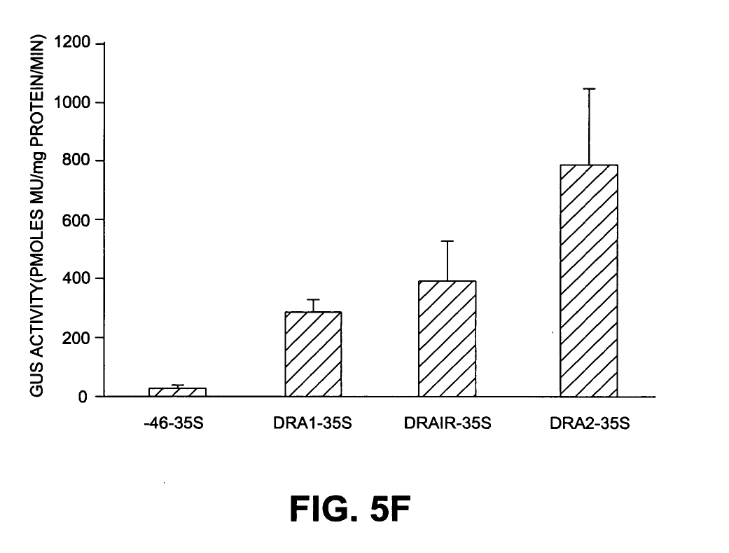

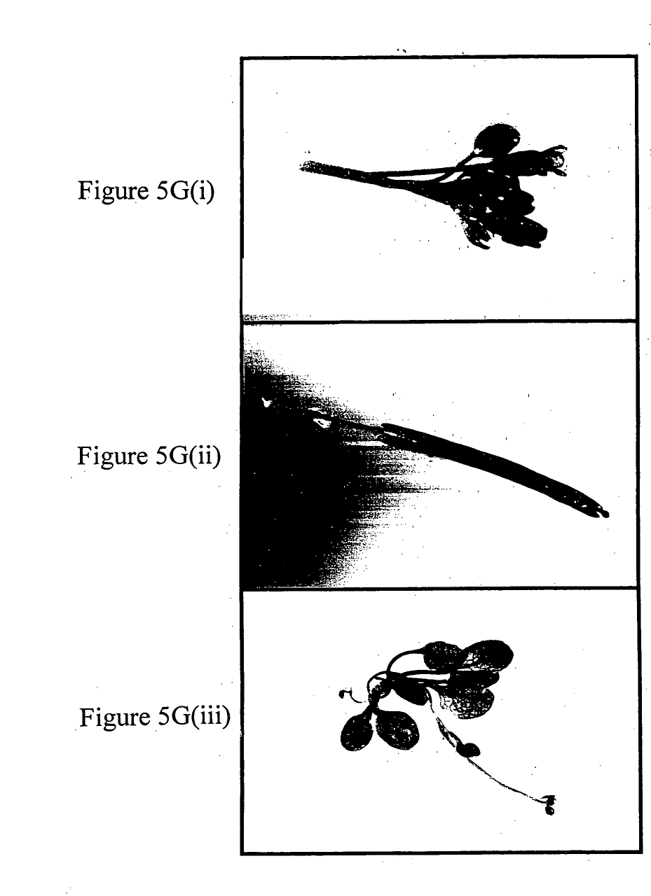

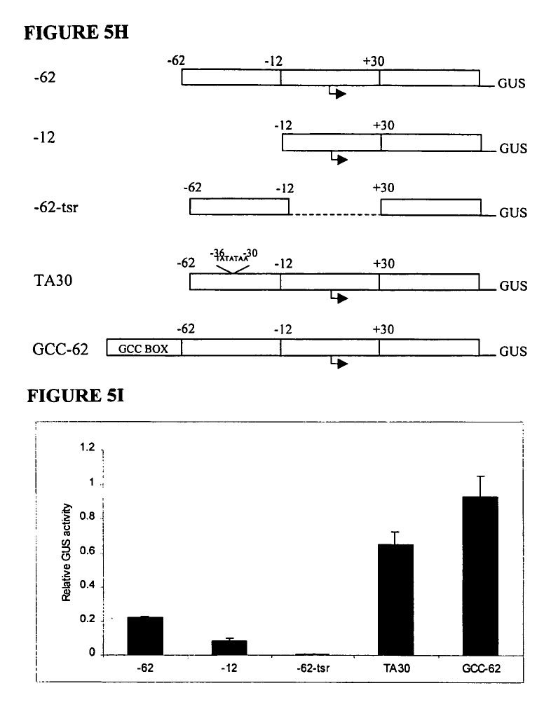

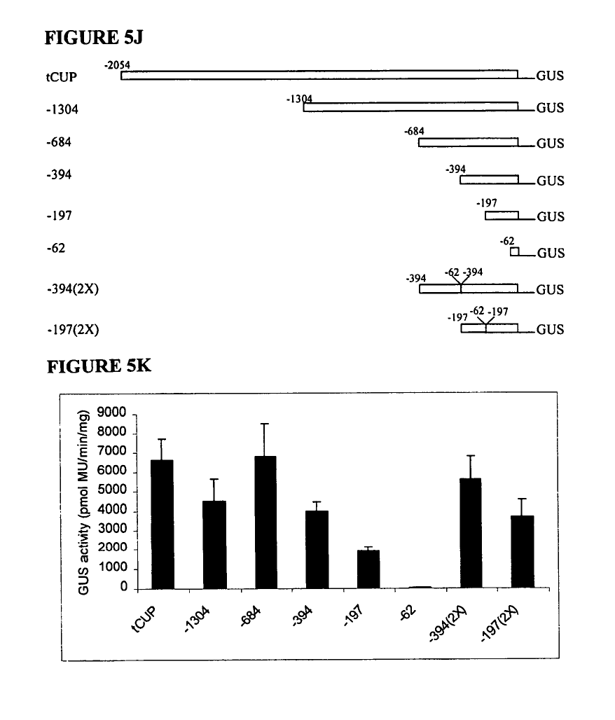

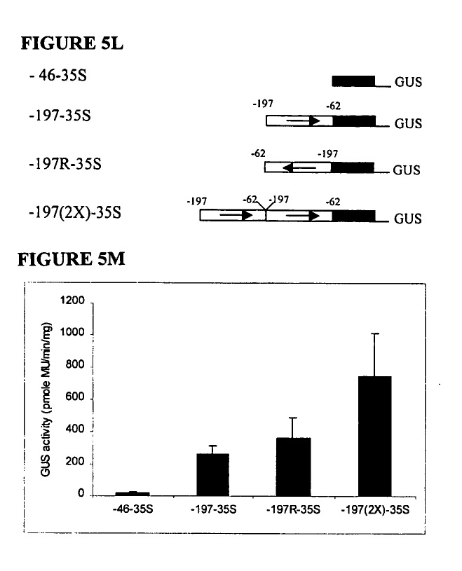

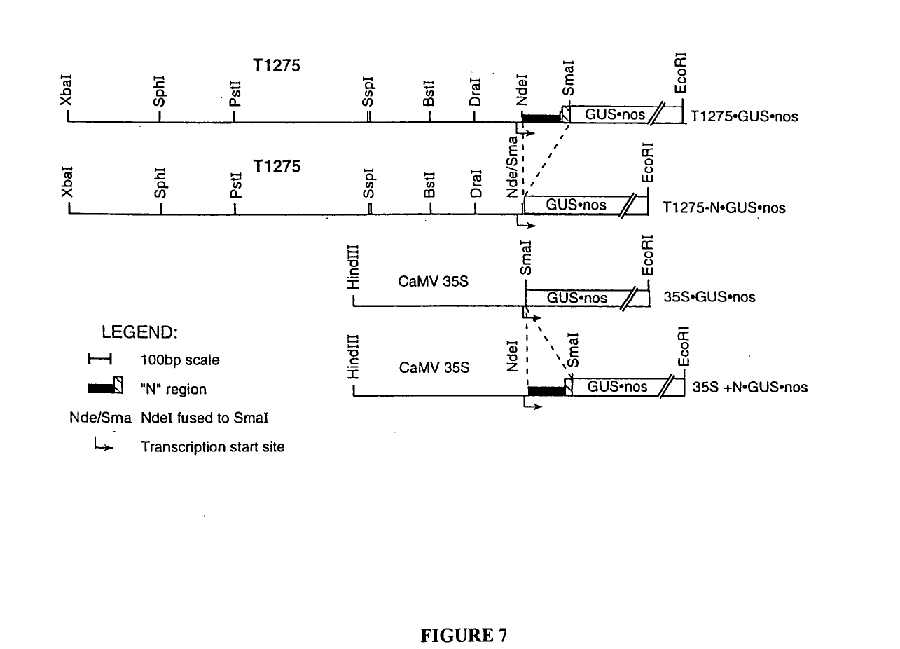

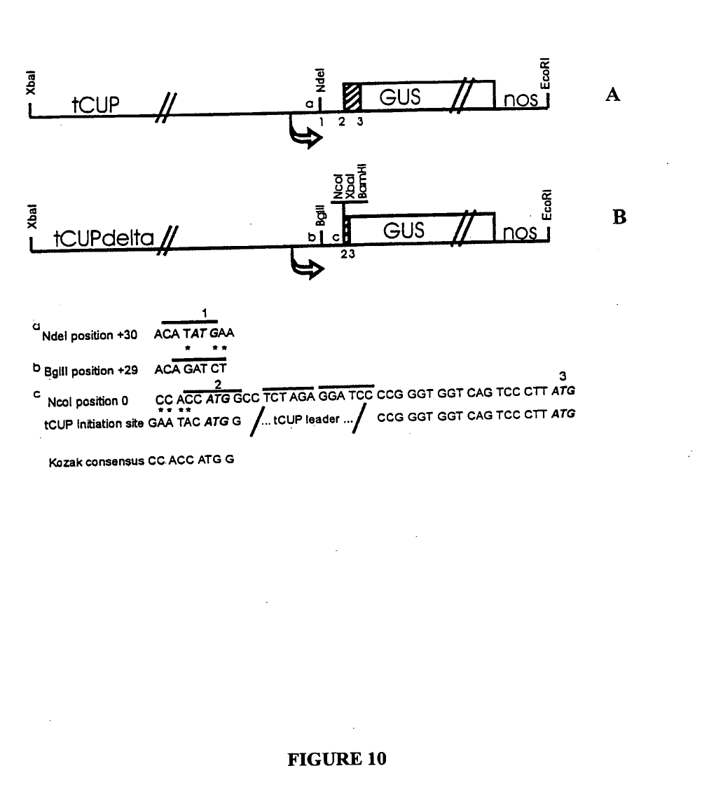

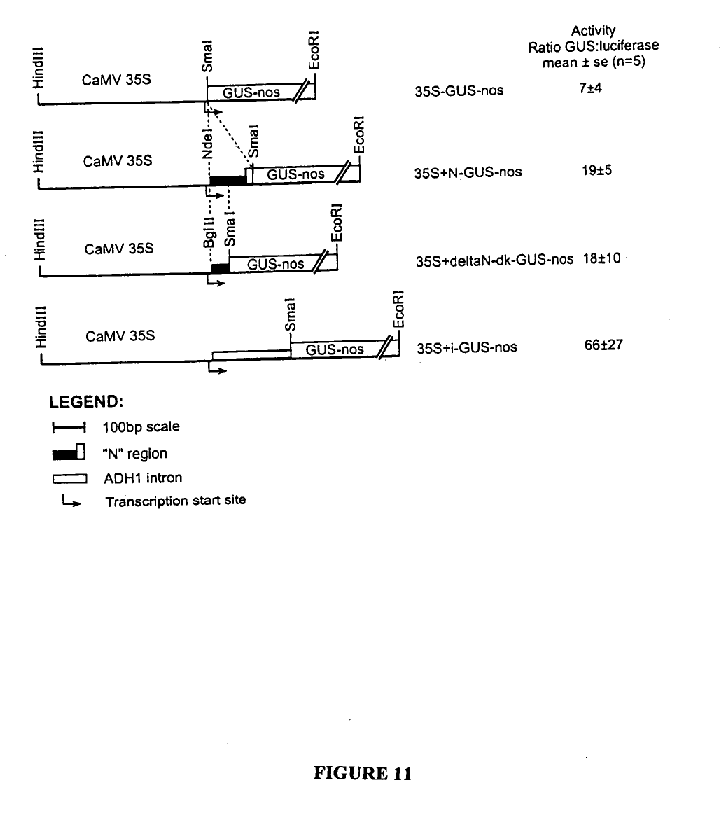

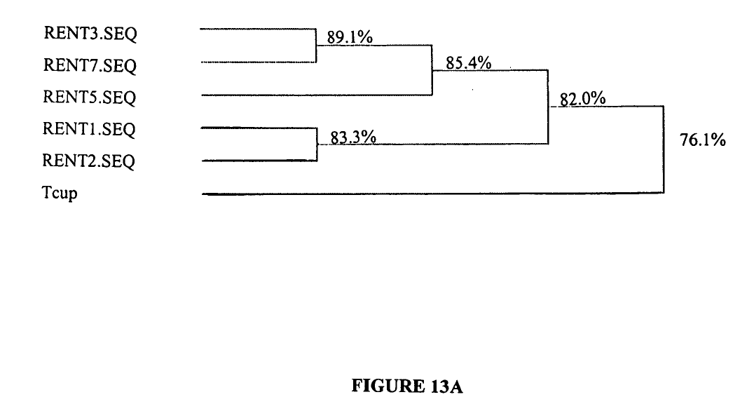

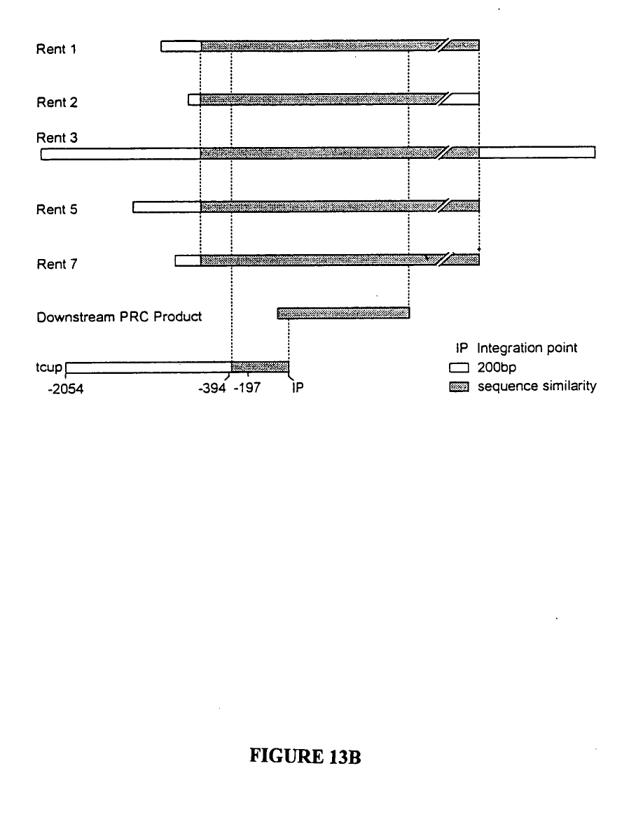

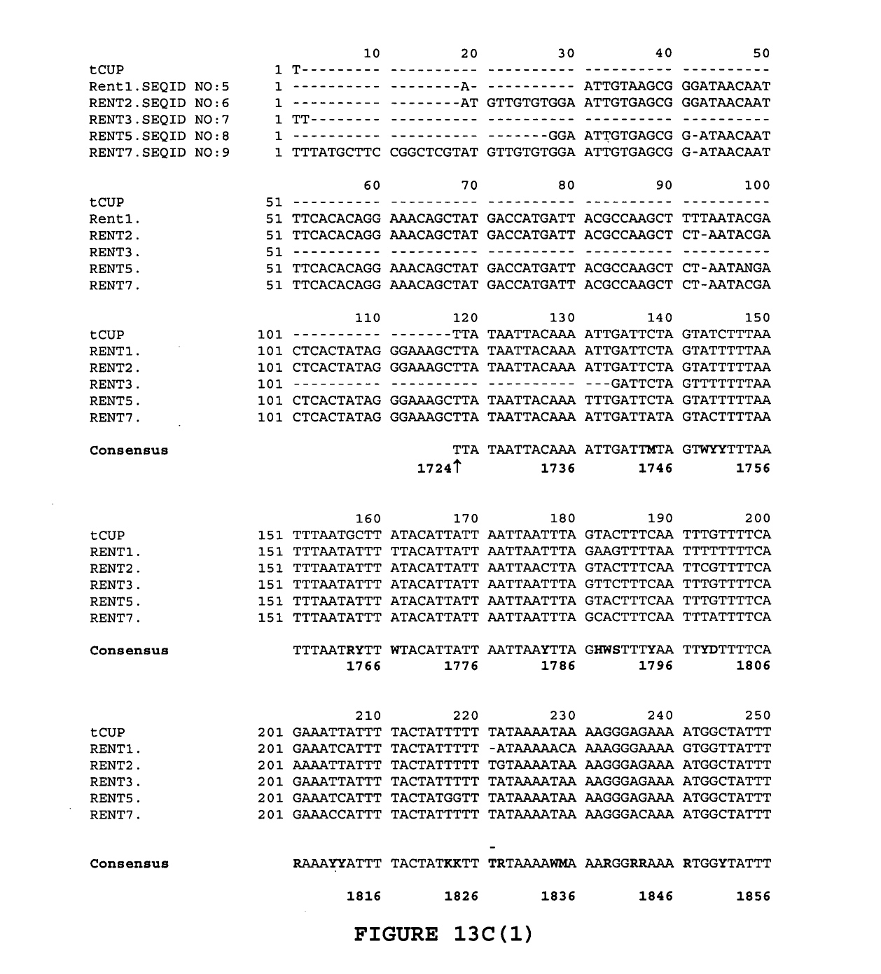

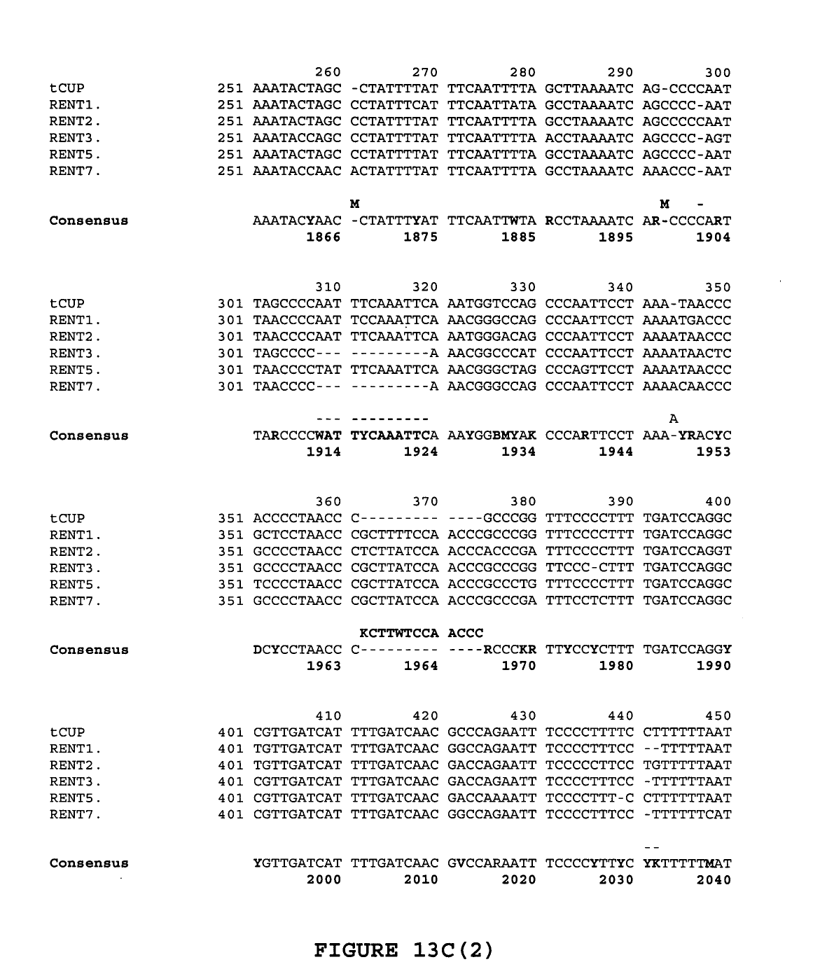

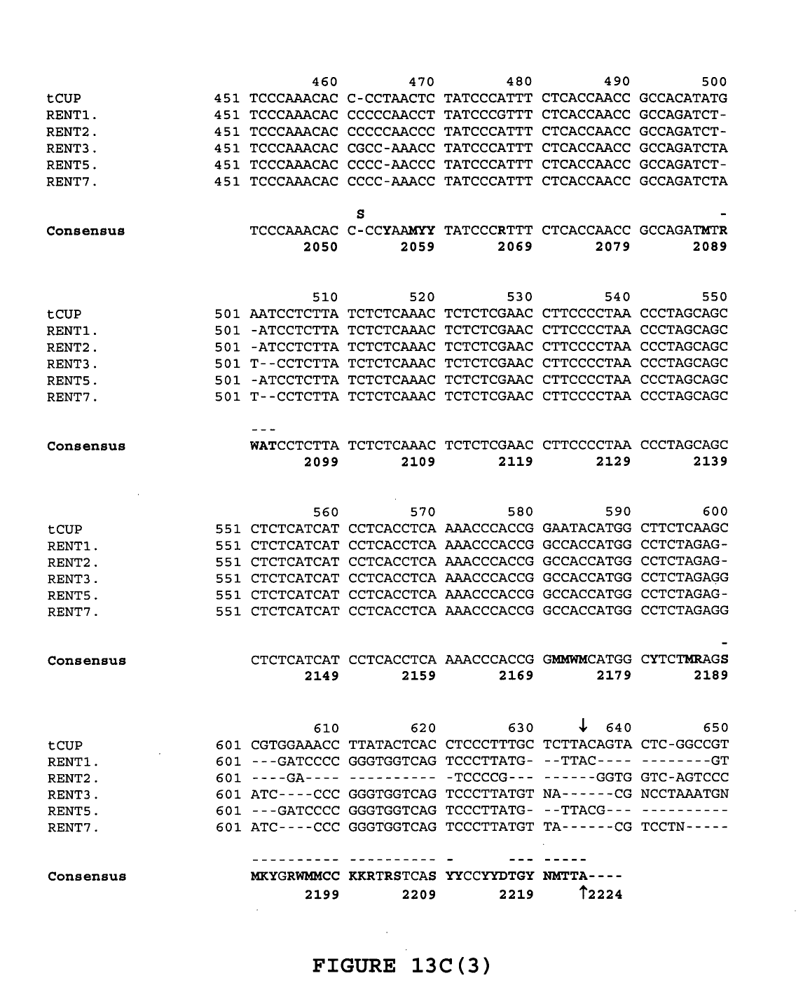

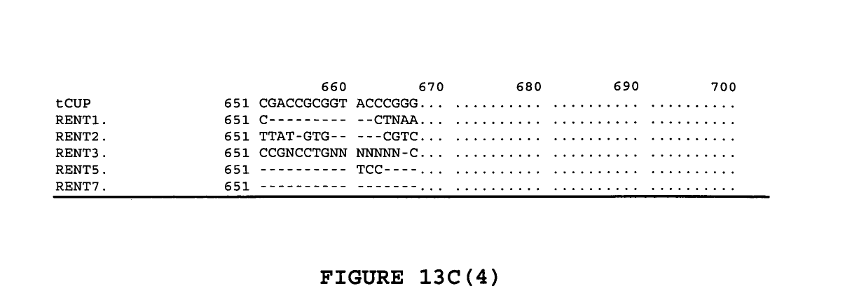

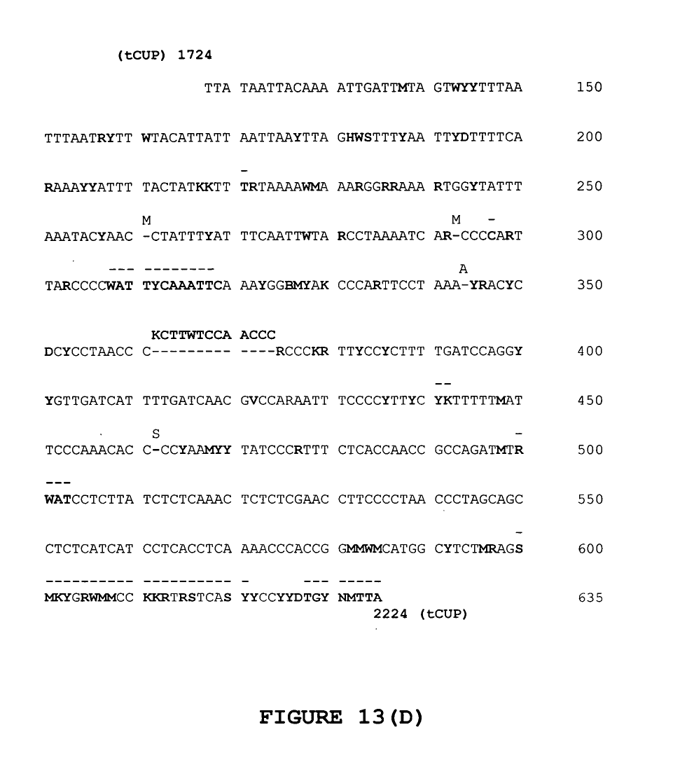

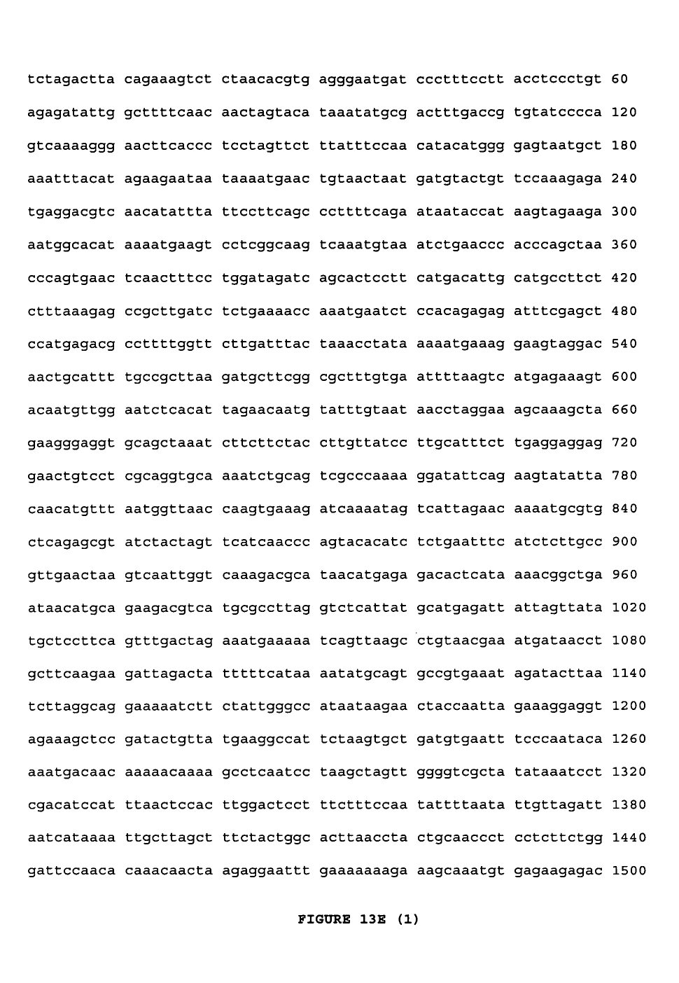

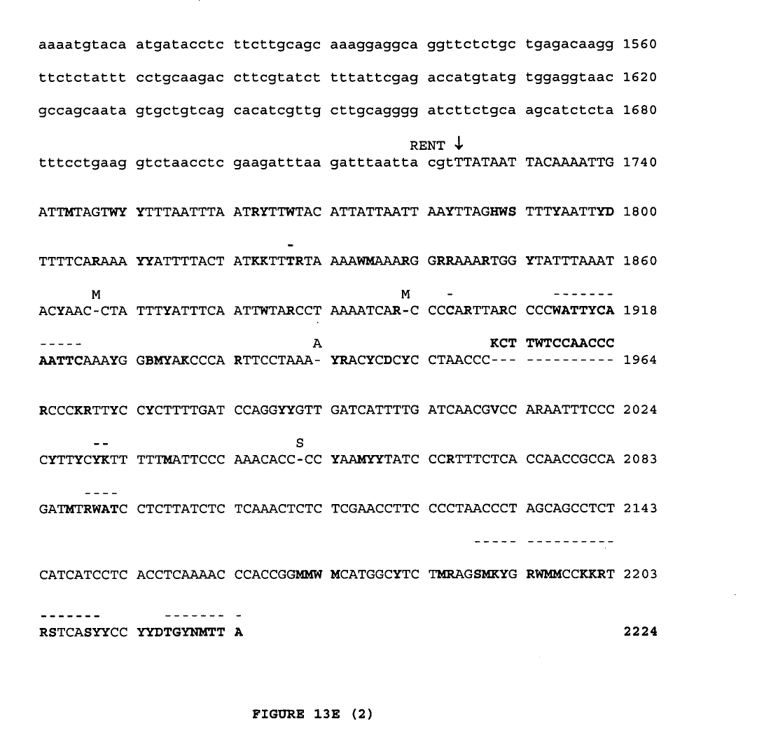

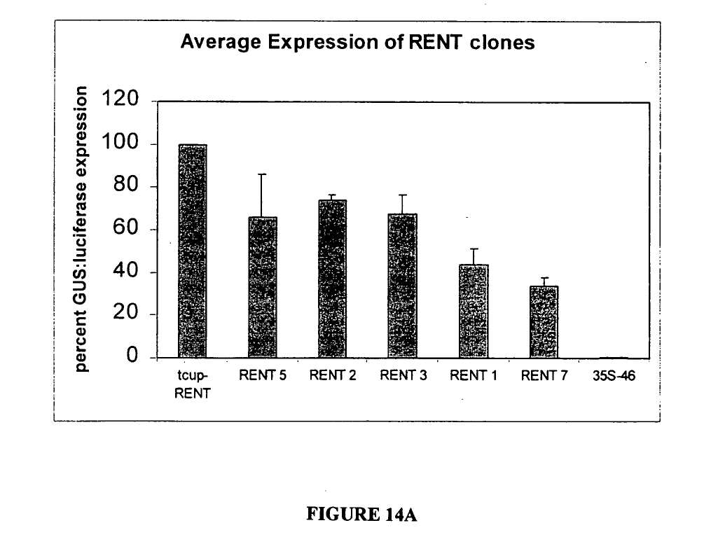

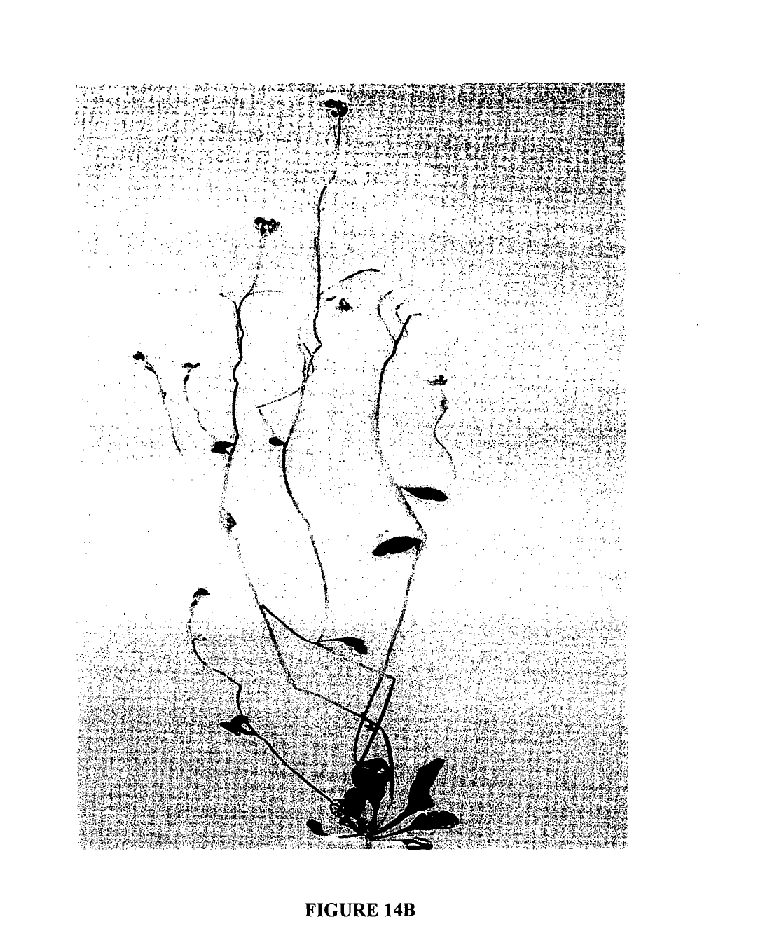

Cloning and deletion analysis of the GUS fusions in these plants revealed that one or more than one regulatory region was located in the plant DNA proximal to the GUS gene. In T1275, a regulatory region was identified within an XbaI-SmaI fragment that exhibits constitutive activity in all organs, tissues and plants tested. This constitutive regulatory element, is referred to as T1275, or tCUP herein (SEQ ID NO's: 1 or 22), and comprises several other regulatory elements throughout the sequence, and that exhibit regulatory region activity as defined herein, for example:
a minimal promoter region between DraI and NdeI sites (1875-2084 of SEQ ID NO's: 1 and 22), also referred to as a core promoter element; see FIG. 5C “−197-GUS-nos”, and Table 6;
negative regulatory elements between 1040-1370 of SEQ ID NO's: 1 and 22 (“−1304 to −684”; see FIGS. 5J and K, where activity obtained for “tCUP” and “−684” are each above that of the activity obtained for “−1304”);
a transcriptional enhancer between BstYI and DraI sites (1660-1875 of SEQ ID NO's: 1 and 22), also referred to as a BstYI-DraI fragment; see FIG. 5C e.g. “−394 GUS-nos”, and Table 6);
a translational enhancer regulatory element between NdeI and SmaI sites (2084-2224 of SEQ ID NO's: 1 and 22) see FIG. 5B (+30-GUS-nos), FIG. 7 (T1275-GUS-nos; 35S-GUS-nos) and Tables 7-13. This fragment is also referred to as “N” herein. Also see FIG. 11 (compare the activity of 35S+N-GUS-nos, comprising the NdeI-SmaI fragment, with that of 35S-GUS-nos, lacking the NdeI-SmaI fragment). A shortened fragment of N comprising nucleotides 2091-2170 of SEQ ID NO's:1 and 22 (presented in SEQ ID NO:2; also referred to as dN, deltaN, tCUP delta), ΔN^{M} (a fragment that lacks a Kozak sequence; SEQ ID NO:4), or a fragment that comprises a Kozak sequence (FIG. 10, SEQ ID NO:3) also exhibit enhancer regulatory element activity.
an enhancer element between 1660-1992 of SEQ ID NO's: 1 and 22 (fragment between BstYI (“−394”) and “−62”), see FIG. 5D (see Bst1-GUS; Bst1-35S, and tandem fragments: Bst2-GUS, Bst2-35S);
a transcriptional enhancer between 1875-1992 of SEQ ID NO's: 1 and 22 (fragment between Dra1 (“−197”) and “−62”), see FIG. 5D (Dra1-GUS; Dra2-GUS; Dra1-35S; Dra2-35S), and FIGS. 5E-G (Dra1-35S; Dra2-35S); and
members of the RENT family exhibit greater than 75% sequence identity with nucleotides 1724-2224 of SEQ ID NO: 1, or more preferably, from about 77% to 92% sequence identity with nucleotides 1724-2224 of SEQ ID NO: 1 (see FIGS. 13C, 13D and 14A. This region includes several of the regulatory elements identified above including the minimal promoter between DraI and NdeI sites (1875-2086 of SEQ ID NO: 1) and the translational enhancer between NdeI and SmaI sites (2084-2224 of SEQ ID NO: 1). The consensus sequence for members of the RENT family (SEQ ID NO's: 21 and 22) is presented in FIGS. 13(C)-13(E).

Therefore, the present invention provides one or more than one regulatory region obtained from T1275 (tCUP; SEQ ID NO's: 1 or 22), wherein the regulatory region may comprise:
the full length sequence of SEQ ID NO: 1, SEQ ID NO:21, or SEQ ID NO:22;
a nucleotide sequence that hybridizes to SEQ ID NO: 1, SEQ ID NO:21, or SEQ ID NO:22;
a nucleotide sequence that hybridizes to the compliment of SEQ ID NO: 1, SEQ ID NO:21, or SEQ ID NO:22;
a fragment of SEQ ID NO:1, SEQ ID NO:21 or SEQ ID NO:22, or
a nucleotide sequence that hybridizes to a fragment of SEQ ID NO: 1, SEQ ID NO:21, or SEQ ID NO:22,
   wherein the nucleotide sequence exhibits regulatory element activity, or is capable of mediating transcriptional efficiency of a transcript encoding a gene of interest that is operatively linked thereto.

By a nucleotide sequence exhibiting regulatory element activity it is meant that the nucleotide sequence, when operatively linked with a coding sequence of interest, regulates, modifies or mediates the expression of the coding sequence. For example, a nucleotide sequence exhibiting regulatory element activity may function as a promoter, a core promoter, a constitutive regulatory element, a negative element or silencer (i.e. elements that decrease promoter activity), or a transcriptional or translational enhancer, thereby regulating, modifying or mediating expression of a coding region of interest that may be operatively linked thereto. Hybridization condition may be selected from the group consisting of:
hybridizing overnight in a solution comprising 7% SDS, 0.5M NaPO4 buffer at pH 7.2, and 10 mM EDTA at 65° C. and washing for one hour at 60° C. in a solution comprising 0.1×SSC and 0.1% SDS;
hybridizing overnight in a solution comprising 7% SDS, 0.5M NaPO4 buffer at pH 7.2, and 10 mM EDTA at 65° C. and washing for one hour at 65° C. in a solution comprising 2×SSC and 0.1% SDS; and
hybridizing overnight in a solution comprising 4×SSC at 65° C. and washing one hour in 0.1×SSC at 65° C.

Furthermore, the present invention exemplifies the use of one or more probes, for example but not limited to nucleotides 1660-2224 of SEQ ID NO: 1 (BstYI-SmaI fragment), that may be used identify members of the RENT family of sequences (see Examples “RENT Repetitive Element from N. tabacum family of repetitive elements” in the Examples).

However, it is to be understood that other portions of the isolated disclosed regulatory elements within T1275 (tCUP) may also exhibit activities in directing organ specificity, tissue specificity, or a combination thereof, or temporal activity, or developmental activity, or a combination thereof, or other regulatory attributes including, negative regulatory elements, enhancer sequences, or post transcriptional regulatory elements, including sequences that affect stability of the transcription or initiation complexes or stability of the transcript. The full-length nucleotide sequence of the T1275 (tCUP) regulatory region is provided in SEQ ID NO: 1. Nucleotide sequences that exhibit from about 75% sequence identity with nucleotides from about 1724 to 2224 of the T1274 regulatory region (SEQ ID NO: 1), and that exhibit regulatory element activity, are also disclosed. These nucleotide sequences include members of the RENT family of nucleotide sequences (see FIG. 13C), and when operatively linked with a coding region of interest, drive the expression of the coding region of interest (see FIG. 14(A)).

Thus, the present invention includes, but is not limited to one or more than one regulatory element obtained from plants that is capable of conferring, mediating, modifying, reducing, or enhancing expression upon a coding region of interest operatively linked therewith. Furthermore, the present invention includes one or more than one regulatory element obtained from a plant that is capable of mediating the translational efficiency of a transcript produced from a coding region of interest linked in operative association therewith. It is to be understood that the regulatory elements of the present invention may also be used in combination with other regulatory elements, either cryptic or otherwise, such as promoters, enhancers, or fragments thereof, and the like.

Furthermore, the present invention provides an isolated plant constitutive regulatory element. This regulatory element may be characterized in that:
it directs expression in a variety of plant tissues and organs, for example, the ovary, flower, immature embryo, mature embryo, seed, stem, leaf, root and cultured tissues;
it lacks a TATA box;
it is not detected in untransformed soybean, potato, sunflower, Arabidopsis, B. napus, or B. oleracea, corn, wheat, black spruce, by Southern analysis under the following conditions: 4×SSC at 65° C. overnight (from 12-18 hours), followed by washing in 0.1×SSC at 65° C. for an hour; and
it is a member of a large family of repetitive elements (RENT).

The regulatory element described herein is a member of a large family of repetitive elements identified within the Nicotiana tabacum SR1 genome that exhibits greater than about 75%, and preferably from about 77% to about 90% sequence similarity to fragment of approximately 532 bp of SEQ ID NO: 1 (including nucleotides 1724 to 2224; see FIGS. 13(A) and (C); the sequence of tCUP in FIG. 13(C) includes the tDNA portion of the T1275 sequence which comprise nucleotides 635-667 of FIG. 13(C)). This family of repetitive elements has been termed RENT (Repetitive Element Nicotiana tabacum). The approximately 532 bp fragment of SEQ ID NO: 1, and related nucleotide sequences as determined within the RENT family (SEQ ID NO's: 5 to 9), exhibit regulatory element activity and are capable of directing GUS expression in a range of plants. The RENT consensus sequence is provided in FIGS. 13(C)-(E) and in SEQ ID NO's:21 and 22.

This invention is also directed to a regulatory element that comprises a nucleotide sequence of at least 18 contiguous base pairs of SEQ ID NO's: 1, 5, 6, 7, 8, 9, 21 or 22. Oligonucleotides of 18 bp or more are useful in constructing heterologous regulatory elements that comprise fragments of the regulatory element as defined in SEQ ID NO's:1, 5, 6, 7, 8, 9, 21, or 22. The use of such heterologous regulatory elements is well established in the literature. For example, fragments of specific elements within the 35S CaMV promoter have been duplicated or combined with other promoter fragments to produce chimeric promoters with desired properties (e.g. U.S. Pat. No. 5,491,288; U.S. Pat. No. 5,424,200; U.S. Pat. No. 5,322,938; U.S. Pat. No. 5,196,525; U.S. Pat. No. 5,164,316). Oligonucleotides of 18 bps or longer are useful as probes or PCR primers in identifying or amplifying related DNA or RNA sequences in other tissues or organisms. Furthermore, oligonucleotides of 18 bps or more are useful in identifying sequences homologous to those identified within SEQ ID NO's:1, 5 to 9, 21 or 22 for example, but not limited to, the RENT family of elements, as described herein.

By “regulatory element” or “regulatory region”, it is meant a portion of nucleic acid typically, but not always, upstream of a gene, and may be comprised of either DNA or RNA, or both DNA and RNA. The regulatory elements of the present invention include those which are capable of mediating organ specificity, or controlling developmental or temporal gene activation. Furthermore, “regulatory element” includes promoter elements, core promoter elements, elements that are inducible in response to an external stimulus, elements that are activated constitutively, or elements that decrease or increase promoter activity such as negative regulatory elements or transcriptional enhancers, respectively. By a nucleotide sequence exhibiting regulatory element activity it is meant that the nucleotide sequence when operatively linked with a coding sequence of interest functions as a promoter, a core promoter, a constitutive regulatory element, a negative element or silencer (i.e. elements that decrease promoter activity), or a transcriptional or translational enhancer.

By “operatively linked” it is meant that the particular sequences, for example a regulatory element and a coding region of interest, interact either directly or indirectly to carry out an intended function, such as mediation or modulation of gene expression. The interaction of operatively linked sequences may, for example, be mediated by proteins that interact with the operatively linked sequences.

Regulatory elements as used herein, also includes elements that are active following transcription initiation or transcription, for example, regulatory elements that modulate gene expression such as translational and transcriptional enhancers, translational and transcriptional repressors, and mRNA stability or instability determinants. In the context of this disclosure, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements (as listed above) that modify gene expression. It is to be understood that nucleotide sequences, located within introns, or 3′ of the coding region sequence may also contribute to the regulation of expression of a coding region of interest. A regulatory element may also include those elements located downstream (3′) to the site of transcription initiation, or within transcribed regions, or both. In the context of the present invention a post-transcriptional regulatory element may include elements that are active following transcription initiation, for example translational and transcriptional enhancers, translational and transcriptional repressors, and mRNA stability determinants.

The regulatory elements, or fragments thereof, of the present invention may be operatively associated (operatively linked) with heterologous regulatory elements or promoters in order to modulate the activity of the heterologous regulatory element. Such modulation includes enhancing or repressing transcriptional activity of the heterologous regulatory element, modulating post-transcriptional events, or both enhancing or repressing transcriptional activity of the heterologous regulatory element and modulating post-transcriptional events. For example, one or more regulatory elements, or fragments thereof, of the present invention may be operatively associated with constitutive, inducible, tissue specific promoters or fragment thereof, or fragments of regulatory elements, for example, but not limited to TATA or GC sequences may be operatively associated with the regulatory elements of the present invention, to modulate the activity of such promoters within plant, insect, fungi, bacterial, yeast, or animal cells.

There are generally two types of promoters, inducible and constitutive promoters. An inducible promoter is a promoter that is capable of directly or indirectly activating transcription of one or more DNA sequences or genes in response to an inducer. In the absence of an inducer the DNA sequences or genes will not be transcribed. Typically the protein factor that binds specifically to an inducible promoter to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible promoter may be exposed to an inducer by externally applying the inducer to the cell or plant such as by spraying, watering, heating or similar methods.

A constitutive promoter directs the expression of a gene throughout the various parts of a plant and continuously throughout plant development. Examples of known constitutive promoters include those associated with the CaMV 35S transcript. (Odell et al., 1985, Nature, 313: 810-812), the rice actin 1 (Zhang et al, 1991, Plant Cell, 3: 1155-1165) and triosephosphate isomerase 1 (Xu et al, 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), and the tobacco translational initiation factor 4A gene (Mandel et al, 1995 Plant Mol. Biol. 29: 995-1004). The present invention is directed to a DNA sequence which contains a regulatory element capable of directing the expression of a gene. Preferably the regulatory element is a constitutive regulatory element isolated from N. tabacum.

The term “constitutive” as used herein does not necessarily indicate that a gene is expressed at the same level in all cell types, but that the gene is expressed in a wide range of cell types, although some variation in abundance is often observed.

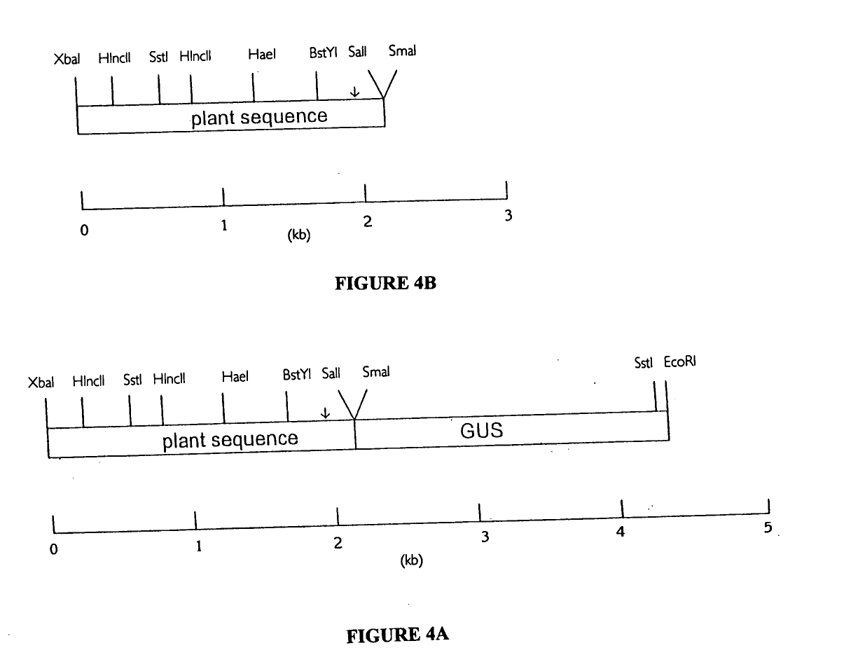

An example, which is not to be considered limiting in any manner, of a regulatory element of the present invention includes a constitutive regulatory element obtained from the plant T1275, as described herein and analogues or fragments thereof, or a nucleic acid fragment localized between XbaI-SmaI, as identified by the restriction map of FIG. 4(B) or a fragment thereof. Furthermore, the regulatory element may be defined as a nucleic acid fragment localized between XbaI-SmaI as identified by the restriction map of FIG. 5(C) or a fragment thereof. The regulatory element may also be defined by a nucleotide sequence comprising at least an 18 bp fragment of the regulatory region defined in SEQ ID NO's: 1, 5, 6, 7, 8, 9, 21 or 22 The regulatory element may also be defined by a nucleic acid comprising from about 70%, preferably greater than about 75%, nucleotide sequence similarity to the nucleotide sequence of SEQ ID NO's:1, 5, 6, 7, 8, 9, 21 or 22 or a fragment thereof, or by a nucleic acid substantially homologous to the nucleotide sequence of SEQ ID NO's: 1, 5, 6, 7, 8, 9, 21 or 22 or a fragment thereof, wherein the nucleic acid exhibits regulatory element activity.

Another regulatory element of the present invention includes, but is not limited to, a post-transcriptional or translational enhancer regulatory element localized between NdeI-SmaI (see FIGS. 5(A), (B) or (C), FIG. 7, and FIG. 11), or the post-transcriptional or translational enhancer regulatory element may comprise the nucleotide sequence as defined by nucleotides 2084-2224 of SEQ ID NO: 1 or an analog thereof, or the element may comprise 70% similarity to the nucleotide sequence of nucleotides 2084-2224 of SEQ ID NO: 1 (i.e. a portion of the NdeI-SmaI fragment from NdeI to the integration point of T1275 at nucleotide 2224).

Furthermore, other regulatory elements of the present invention include negative regulatory elements (for example located within an XbaI-BstYI fragment as defined by FIG. 5(C), and described in more detail below), a transcriptional enhancer localized within the BstYI-DraI fragment of FIG. 5(C), a core regulatory element located within the DraI-NdeI fragment of FIG. 5(C), or a regulatory element or post-transcriptional element downstream of the transcriptional start site.

A further regulatory element of the present invention includes an enhancer element within the −394 to −62 fragment of T1275 (nucleotides 1660 to 1992 of SEQ ID NO: 1). This fragment may also be duplicated and fused to a regulatory region, for example a core promoter, producing an increase in the activity of the regulatory region (see FIG. 5(D)). A portion of the −394 to −62 fragment of T1275 (tCUP), from nucleotides 1724-1992 of SEQ ID NO: 1 or 22 exhibits substantial homology with other members of the RENT family of repetitive sequences (FIGS. 13(A)-(C)). The homologous fragment present within the RENT family of sequences also exhibit regulatory element activity (FIG. 14(A)) and are active in a range of plants, and direct the constitutive expression of a coding region of interest throughout a plant (FIG. 14(B)).

Therefore, the present invention also provides for a chimeric nucleic acid construct comprising a regulatory element in operative association with a coding region of interest, the regulatory element comprising nucleotides 1660-1992 of SEQ ID NO: 1 (or SEQ ID NO:22), or a duplicate thereof.

Another regulatory element of the present invention includes, but is not limited to, a post-transcriptional or translational enhancer regulatory element localized between NdeI-SmaI (see FIG. 7, nucleotides 2084-2224 of SEQ ID NO: 1 or 22; or nucleotides 1-188 of SEQ ID NO:2), also referred to as “N”. The post-transcriptional or translational enhancer regulatory element may also comprise the nucleotide sequence as defined by nucleotides 1-141 of SEQ ID NO:2 (nucleotides 2084-2224 of SEQ ID NO: 1 or 22) or an analog thereof, or the element may comprise 70% similarity (sequence identity) to the nucleotide sequence of nucleotides 1-141 of SEQ ID NO:2 (nucleotides 2084-2224 of SEQ ID NO: 1 or 22). This regulatory element also exhibits substantial homology with members of the RENT family of repetitive elements (see FIG. 13(C); nucleotides 495-635 or nucleotides 2084-2224 of tCUP).

A shortened fragment of the NdeI-SmaI fragment, referred to as ΔN, dN, deltaN, or tCUP delta, is also characterized within the present invention. ΔN was prepared by mutagenesis replacing the out of frame ATG (located at nucleotides 2087-2089, SEQ ID NO: 1) within the NdeI-SmaI fragment (see FIG. 10). ΔN constructs with (SEQ ID NO:3) or without (SEQ ID NO:4) a Kozak consensus sequence was also characterized (Tables 10, and 12) and found to exhibit enhancer activity. Therefore, other cryptic regulatory elements of the present invention include, but are not limited to, post-transcriptional or translational enhancer regulatory elements localized at nucleotides 1-97 of SEQ ID NO's:3 and nucleotides 1-86 of SEQ ID NO's: 3 or 4. These post-transcriptional or translational enhancer regulatory elements may comprise the nucleotide sequence as defined by nucleotides 1-86 of SEQ ID NO's:3 or 4 (nucleotides 2091-2170 of SEQ ID NO:1) or an analog thereof, or the element may comprise 70% similarity to the nucleotide sequence of nucleotides 1-86 of SEQ ID NO's:3 or 4 (nucleotides 2091-2170 of SEQ ID NO:1). Furthermore, these regulatory elements may comprise the nucleotide sequence as defined by nucleotides 1-97 of SEQ ID NO:3 and comprising a Kozack sequence or an analog thereof, or the element may comprise 70% similarity to the nucleotide sequence of nucleotides 1-97 of SEQ ID NO:3.

Furthermore, other regulatory elements of the present invention include negative regulatory elements (for example located within an XbaI-BstYI fragment as defined by FIG. 5(C); nucleotides 1-1660 of SEQ ID NO: 1), a transcriptional enhancer localized within the BstYI-DraI fragment of FIG. 5(C) (nucleotides 1660-1875 of SEQ ID NO: 1), a core promoter element located within the DraI-NdeI fragment of FIG. 5(C) (nucleotides 1875-2084 of SEQ ID NO: 1 or 22), a transcriptional enhancer within the Dra1 to −62 fragment (nucleotides 1875-1992 of SEQ ID NO: 1 or 22; FIGS. 5(D) to (G)), or a regulatory element or post-transcriptional element downstream of the transcriptional start site, for example but not limited to the NdeI-SmaI fragment (nucleotides 1-188 of SEQ ID NO2) and derivatives and fragments thereof (for example nucleotides 1-141 of SEQ ID NO:2), including ΔN (nucleotides 1-129 or 1-97 of SEQ ID NO:3, ΔN^{M} (nucleotides 1-119 or 1-86 SEQ ID NO:4), and nucleotides 1-86 of SEQ ID NO:3 or 4 (nucleotides 2084 to 2170 of SEQ ID NO:1).

The following non-limiting list of fragments of SEQ ID NO: 1 or 22 have been characterized and their utility demonstrated herein, nucleotides:

1660-1992 (“−394” to “−62” fragment) enhances expression of the −46 minimal promoter of 35S, and a fragment of T1275 (see Bst1-GUS; Bst1-35S, Bst2-GUS, Bst2-35S, of FIG. 5D);
1660-1875 (BstYI-DraI fragment; see FIG. 5C; and Table 6; −394 GUS-nos) exhibits enhancer activity;
1660-2224 (BstYI-SmaI fragment; see FIG. 5C; and Tables 5 and 6; −394-GUS-nos) also exhibits enhancer activity;
1724-2224 (FIG. 13C, and FIG. 14A, “tCUP RENT”) exhibits regulatory element activity and comprises several regulatory elements (core promoter element and an translational enhancer element). Nucleic acid sequences that hybridize to nucleotides 1724-2224 under stringent hybridization conditions and that exhibit one or more than one regulatory element activity, or nucleic acid sequences that exhibit greater than 75% sequence identity with nucleotides 1724-2224 and that exhibit one or more than one regulatory element activities, are members of the RENT family (SEQ ID NO's:21 and 22);
1875-2084 (DraI-NdeI fragment; core promoter element), see FIG. 5C and Table 6 (−197-GUS-nos);
1875-1992 (DraI—“−62” fragment) This fragment is shown to enhance expression of the −46 minimal promoter of 35S, and a fragment of T1275, as shown in FIG. 5D (see Dra1-GUS; Dra2-GUS; Dra1-35S; Dra2-35S), and FIGS. 5E-G (Dra1-35S; Dra2-35S), and functions as a transcriptional enhancer;
2084-2224 (NdeI-SmaI fragment, or “N”; Tables 10-12, FIG. 5B (+30-GUS-nos), FIG. 7 (T1275-GUS-nos; 35S-GUS-nos), and FIG. 11 (35S+N-GUS-nos) exhibits translational regulatory element activity; and
2091-2170 (ΔN; see Tables 10-12) exhibits translational enhancer activity.

Therefore, the present invention is directed to an isolated nucleic acid sequence comprising a regulatory element selected from the group consisting of a nucleotide sequence:
defined by nucleotides 1-1660 of SEQ ID NO: 1 or 22 (XbaI-BstYI),
defined by nucleotides 1660-1992 of SEQ ID NO:1 or 22 (BstYI to −62),
defined by nucleotides 1660-1875 of SEQ ID NO:1 or 22 (BstYI-DraI),
defined by nucleotides 1660-2224 of SEQ ID NO:1 or 22 (BstYI-SmaI),
defined by nucleotides 1724-2224 of SEQ ID NO:1 or 22 (RENT),
defined by nucleotides 1875-2084 of SEQ ID NO:1 or 22 (DraI-NdeI),
defined by nucleotides 1875-1992 of SEQ ID NO:1 or 22(Dra1 to −62),
defined by nucleotides 2084-2224 of SEQ ID NO:1 or 22 (NdeI-SmaI),
defined by nucleotides 2091-2170 of SEQ ID NO:1 or 22 (N),
defined by nucleotides 1992-2042 of SEQ ID NO:1 or 22 (−62 to −12),
defined by nucleotides 415-2224 of SEQ ID NO:1 or 22 (SphI-Sma1),
defined by nucleotides 1040-2224 of SEQ ID NO:1 or 22 (PstI-Sma1), and
defined by nucleotides 1370-2224 of SEQ ID NO:1 or 22 (SspI-Sma1).

The present invention also provides an isolated nucleic acid sequence comprising a regulatory element selected from the group consisting of a nucleotide sequence:
that hybridizes to nucleotides 1-1660 of SEQ ID NO: 1 or 22 or a compliment thereof,
that hybridizes to nucleotides 1660-1992 of SEQ ID NO: 1 or 22 or a compliment thereof,
that hybridizes to nucleotides 1660-1875 of SEQ ID NO:1 or 22 or a compliment thereof,
that hybridizes to nucleotides 1724-2224 of SEQ ID NO: 1 or 22 or a compliment thereof,
that hybridizes to nucleotides 1875-2084 of SEQ ID NO: 1 or 22 or a compliment thereof,
that hybridizes to nucleotides 1875-2224 of SEQ ID NO: 1 or 22 or a compliment thereof,
that hybridizes to nucleotides 1875-1992 of SEQ ID NO: 1 or 22 or a compliment thereof,
that hybridizes to nucleotides 2084-2224 of SEQ ID NO: 1 or 22 or a compliment thereof, and
that hybridizes to nucleotides 2091-2170 of SEQ ID NO:1 or 22 or a compliment thereof,
   wherein hybridization is under a condition selected from the group consisting of:
hybridizing overnight (16-20 hours) in a solution comprising 7% SDS, 0.5M NaPO4 buffer at pH 7.2, and 10 mM EDTA at 65° C. and washing for one hour at 60° C. in a solution comprising 0.1×SSC and 0.1% SDS;
hybridizing overnight (16-20 hours) in a solution comprising 7% SDS, 0.5M NaPO4 buffer at pH 7.2, and 10 mM EDTA at 65° C. and washing for one hour at 65° C. in a solution comprising 2×SSC and 0.1% SDS; and
hybridizing overnight (16-20 hours) in a solution comprising 4×SSC at 65° C. and washing one hour in 0.1×SSC at 65° C., and
   wherein the regulatory element exhibits regulatory element activity and is capable of mediating the transcriptional or translational efficiency of a transcript encoding a coding region of interest that is operatively linked thereto.

Furthermore, the present invention provides an isolated nucleotide sequence comprising nucleotides defined by the nucleotide sequence of SEQ ID NO:22, or a compliment thereof comprising the following nucleotides at the positions indicated in Table 1a.

TABLE 1a

Identification of nucleotides of the RENT family
and their positions within within SEQ ID NO:22

Nucleotide

| | | | | | | |
|---|---|---|---|---|---|---|
| Position* | tCUP | RENT1 | RENT2 | RENT3 | RENT5 | RENT7 |

| | | | | | | |
|---|---|---|---|---|---|---|
| 1744 | C | C | C | C | C | A |
| 1749 | A | A | A | T | A | A |
| 1750 | T | T | T | T | T | C |
| 1751 | C | T | T | T | T | T |
| 1763 | G | A | A | A | A | A |
| 1764 | C | T | T | T | T | T |
| 1767 | A | T | A | A | A | A |
| 1783 | T | T | C | T | T | T |
| 1788 | T | A | T | T | T | C |
| 1789 | A | A | A | T | A | A |
| 1790 | C | G | C | C | C | C |
| 1794 | C | T | C | C | C | C |
| 1799 | T | T | C | T | T | T |
| 2000 | G | T | G | G | G | A |
| 1807 | G | G | A | G | G | G |
| 1811 | T | T | T | T | T | C |
| 1812 | T | C | T | T | C | C |
| 1823 | T | T | T | T | G | T |
| 1824 | T | T | T | T | G | T |
| 1827 | T | — | T | T | T | T |
| 1828 | A | A | G | A | A | A |
| 1834 | T | A | T | T | T | T |
| 1835 | A | C | A | A | A | A |
| 1839 | G | A | G | G | G | G |
| 1842 | A | G | A | A | A | A |
| 1843 | G | A | G | G | G | G |
| 1847 | A | G | A | A | A | A |
| 1851 | C | T | C | C | C | C |
| 1863 | T | T | T | C | T | C |
| 1866-7 | — | C | C | C | C | A |
| 1873 | T | C | T | T | T | T |
| 1883 | T | A | T | T | T | T |
| 1886 | G | G | G | A | G | G |
| 1897 | G | G | G | G | G | A |
| 1897-8 | — | C | C | C | C | A |
| 1901 | C | — | C | — | — | — |
| 1903 | A | A | A | G | A | A |
| 1907 | G | A | A | G | A | A |
| 1912 | A | A | A | — | T | — |
| 1913 | A | A | A | — | A | — |
| 1914 | T | T | T | — | T | — |
| 1915 | T | T | T | — | T | — |
| 1916 | T | C | T | — | T | — |
| 1917 | C | C | C | — | C | — |
| 1918 | A | A | A | — | A | — |
| 1919 | A | A | A | — | A | — |
| 1920 | A | A | A | — | A | — |
| 1921 | T | T | T | — | T | — |
| 1922 | T | T | T | — | T | — |
| 1923 | C | C | C | — | C | — |
| 1927 | T | C | T | C | C | C |
| 1930 | T | G | G | C | G | G |
| 1931 | C | C | A | C | C | C |
| 1932 | C | C | C | C | T | C |
| 1934 | G | G | G | T | G | G |
| 1939 | A | A | A | A | G | A |
| 1947-8 | — | A | A | A | A | A |
| 1949 | T | T | T | T | T | C |
| 1950 | A | G | A | A | A | A |
| 1952 | C | C | C | T | C | C |
| 1954 | A | G | G | G | T | G |
| 1956 | C | T | C | C | C | C |
| 1964-5 | — | GCTTTTC | TCTTATC | GCTTATC | GCTTATC | GCTTATC |
| | | CAACCC | CAACCC | CAACCC | CAACCC | CAACCC |
| 1966 | G | G | A | G | G | G |
| 1969 | G | G | G | G | T | G |
| 1970 | G | G | A | G | G | A |
| 1973 | T | T | T | C | T | T |
| 1976 | C | C | C | — | C | T |
| 1990 | C | C | T | C | C | C |
| 1991 | C | T | T | C | C | C |
| 2012 | C | G | A | A | A | G |
| 2026 | T | T | C | T | T | T |
| 2029 | T | C | C | C | — | C |
| 2031 | C | — | T | — | C | — |
| 2032 | T | — | G | T | T | T |
| 2038 | A | A | A | A | A | C |
| 2051-2 | — | C | C | G | C | C |
| 2054 | T | C | C | — | — | — |
| 2057 | C | C | C | A | C | A |
| 2058 | T | C | C | C | C | C |
| 2059 | C | T | C | C | C | C |
| 2066 | A | G | A | A | A | A |
| 2086 | A | C | C | C | C | C |
| 2089 | G | — | — | A | — | A |
| 2090 | A | — | — | T | — | T |
| 2091 | A | A | A | — | A | — |
| 2092 | T | T | T | — | T | — |
| 2171 | A | C | C | C | C | C |
| 2172 | A | C | C | C | C | C |
| 2173 | T | A | A | A | A | A |
| 2174 | A | C | C | C | C | C |
| 2181 | T | C | C | C | C | C |
| 2185 | C | A | A | A | A | A |
| 2186 | A | G | G | G | G | G |
| 2189 | C | — | — | G | — | G |
| 2190 | C | — | — | A | — | A |
| 2191 | G | — | — | T | — | T |
| 2192 | T | — | — | C | — | C |
| 2193 | G | G | — | — | G | — |
| 2194 | G | A | G | — | A | — |
| 2195 | A | T | A | — | T | — |
| 2196 | A | C | — | — | C | — |
| 2197 | A | C | — | C | C | C |
| 2198 | C | C | — | C | C | C |
| 2199 | C | C | — | C | C | C |
| 2200 | T | G | — | G | G | G |
| 2201 | T | G | — | G | G | G |
| 2202 | A | G | — | G | G | G |
| 2204 | A | G | — | G | G | G |
| 2205 | C | G | — | G | G | G |
| 2209 | C | G | — | G | G | G |
| 2210 | C | T | — | T | T | T |
| 2211 | T | C | T | C | C | C |
| 2214 | C | T | C | T | T | T |
| 2215 | T | T | C | T | T | T |
| 2216 | T | A | G | A | A | A |
| 2217 | T | T | — | T | T | T |
| 2218 | G | G | — | G | G | G |
| 2219 | C | — | — | T | — | T |
| 2220 | T | — | — | N | — | T |
| 2221 | C | — | — | A | — | A |
| 2222 | T | T | — | — | T | — |
| 2223 | T | T | — | — | T | — |
| 2224 | A | A | — | — | A | — |

*position within SEQ ID NO:22

wherein the nucleotide sequence exhibits regulatory element activity and is capable of conferring or enhancing expression on a coding region of interest linked in operative association therewith.

An “analogue” of the above identified regulatory elements includes any substitution, deletion, or additions to the sequence of a regulatory element provided that said analogue maintains at least one regulatory property associated with the activity of the regulatory element. Such properties include directing organ specificity, tissue specificity, or a combination thereof, or temporal activity, or developmental activity, or a combination thereof, or other regulatory attributes including, negative regulatory elements, enhancer sequences, or sequences that affect stability of the transcription or translation complexes or stability of the transcript.

The present invention is further directed to a chimeric gene construct containing a DNA of interest operatively linked to the regulatory element of the present invention. Any exogenous gene can be used and manipulated according to the present invention to result in the expression of said exogenous gene. A DNA or coding region of interest may include, but is not limited to, a gene encoding a protein, a DNA that is transcribed to produce antisense RNA, or a transcript product that functions in some manner that mediates the expression of other DNAs, for example that results in the co-suppression of other DNAs or the like. A coding region of interest may also include, but is not limited to, a gene that encodes a pharmaceutically active protein, for example growth factors, growth regulators, antibodies, antigens, their derivatives useful for immunization or vaccination and the like. Such proteins include, but are not limited to, interleukins, insulin, G-CSF, GM-CSF, hPG-CSF, M-CSF or combinations thereof, interferons, for example, interferon-α, interferon-β, interferon-τ, blood clotting factors, for example, Factor VIII, Factor IX, or tPA or combinations thereof. A coding region of interest may also encode an industrial enzyme, protein supplement, nutraceutical, or a value-added product for feed, food, or both feed and food use. Examples of such proteins include, but are not limited to proteases, oxidases, phytases, chitinases, invertases, lipases, cellulases, xylanases, enzymes involved in oil biosynthesis etc.

The chimeric gene construct of the present invention can further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon.

Examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes and the small subunit of the ribulose-1,5-bisphosphate carboxylase (ssRUBISCO) gene. The 3′ untranslated region from the structural gene of the present construct can therefore be used to construct chimeric genes for expression in plants.

The chimeric gene construct of the present invention can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene, and can be specifically modified so as to increase translation of the mRNA.

To aid in identification of transformed plant cells, the constructs of this invention may be further manipulated to include plant selectable markers. Useful selectable markers include enzymes which provide for resistance to an antibiotic such as gentamycin, hygromycin, kanamycin, and the like. Similarly, enzymes providing for production of a compound identifiable by colour change such as GUS (β-glucuronidase), or luminescence, such as luciferase are useful.

Also considered part of this invention are transgenic plants, trees, yeast, bacteria, fungi, insect and animal cells containing the chimeric gene construct comprising a regulatory element of the present invention. However, it is to be understood that the regulatory elements of the present invention may also be combined with coding region of interest for expression within a range of host organisms that are amenable to transformation. Such organisms include, but are not limited to:
plants, both monocots and dicots, for example, corn, cereal plants, wheat, barley, oat, tobacco, Brassica, soybean, pea, alfalfa, potato, ginseng, Arabidopsis;
trees, gymnosperms and angiosperms, including both hardwood and softwood trees, for example peach, plum, spruce;
yeast, fungi, insects, animal and bacteria cells.

Methods for the transformation and regeneration of these organisms are established in the art and known to one of skill in the art and the method of obtaining transformed and regenerated plants is not critical to this invention.

In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques.

The constructs of the present invention can be introduced into plant cells using Ti plasmids, Ri plasmids, plant virus vectors, direct DNA transformation, microinjection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579 (1997). The present invention further includes a suitable vector comprising the chimeric gene construct.

When specific sequences are referred to in the present invention, it is understood that these sequences include within their scope sequences that are “substantially homologous” to the specific sequences, or sequences or a compliment of the sequences hybridise to one or more than one nucleotide sequence as defined herein under stringent hybridisation conditions. Sequences are “substantially homologous” when at least about 70%, or more preferably 75% of the nucleotides match over a defined length of the nucleotide sequence providing that such homologous sequences exhibit one or more than one regulatory element activity as disclosed herein. For example which is not to be considered limiting, the RENT family of nucleotide sequences as defined herein exhibits greater than about 75% sequence similarity with a fragment (nucleotides 1724 to 2224) of the nucleotide sequence of SEQ ID NO: 1 or 22. Furthermore, members of the RENT family also hybridise with the nucleotide sequence defined by SEQ ID NO: 1 or 22 under stringent hybridisation conditions and exhibits one or more than one regulatory element activity.

Such a sequence similarity may be determined using a nucleotide sequence comparison program, such as that provided within DNASIS (using, for example but not limited to, the following parameters: GAP penalty 5, #of top diagonals 5, fixed GAP penalty 10, k-tuple 2, floating gap 10, and window size 5). However, other methods of alignment of sequences for comparison are well-known in the art for example the algorithms of Smith & Waterman (Adv. Appl. Math. 2:482, 1981), Needleman & Wunsch (J. Mol. Biol. 48:443, 1970), Pearson & Lipman (Proc. Nat'l. Acad. Sci. USA 85:2444, 1988), and by computerized implementations of these algorithms (GAP, BESTFIT, FASTA, and BLAST, available through the NIH.), or by manual alignment and visual inspection (see, e.g., Current Protocols in Molecular Biology, Ausubel et al., eds. 1995 supplement), or using Southern or Northern hybridization under stringent conditions (see Maniatis et al., in Molecular Cloning (A Laboratory Manual), Cold Spring Harbor Laboratory, 1982) to the nucleotide sequence of SEQ ID NO's:1, 5, 6, 7, 8, 9, 21 or 22 provided that the sequences maintain at least one regulatory property or regulatory element activity, as defined herein. Preferably, sequences that are substantially homologous exhibit at least about 80% and most preferably at least about 90% sequence similarity over a defined length of the molecule.

The DNA sequences of the present invention thus include the DNA sequences of SEQ ID NO's:1, 5, 6, 7, 8, 9 21 or 22, their regulatory regions and fragments thereof, as well as analogues of, or nucleic acid sequences comprising about 70% similarity with the nucleic acids, or fragments thereof, as defined in SEQ ID NO: 1, 5 to 9, 21 and 22. Sequences that are “substantially homologous” include any substitution, deletion, or addition within the sequence.

An example of one such stringent hybridization conditions may be overnight (from about 16-20 hours) hybridization in 4×SSC at 65° C., followed by washing in 0.1×SSC at 65° C. for an hour, or 2 washes in 0.1×SSC at 65° C. each for 20 or 30 minutes. Alternatively an exemplary stringent hybridization condition could be overnight (16-20 hours) in 50% formamide, 4×SSC at 42° C., followed by washing in 0.1×SSC at 65° C. for an hour, or 2 washes in 0.1×SSC at 65° C. each for 20 or 30 minutes, or overnight (16-20 hours), or hybridization in Church aqueous phosphate buffer (7% SDS; 0.5M NaPO₄ buffer pH 7.2; 10 mM EDTA) at 65° C., with 2 washes either at 50° C. in 0.1×SSC, 0.1% SDS for 20 or 30 minutes each, or 2 washes at 65° C. in 2×SSC, 0.1% SDS for 20 or 30 minutes each for unique sequence regions.

Analogues also include those DNA sequences which hybridize to the sequence of SEQ ID NO:1, 5, 6, 7, 8, 9, 21 or 22 or a fragment thereof, under relaxed hybridization conditions, provided that said sequences maintain at least one regulatory property of the activity of the regulatory element. Examples of such relaxed hybridization conditions includes overnight (16-20 hours) hybridization in 4×SSC at 50° C., with 30-40% formamide at 42° C., or 65° C. in 2×SSC, 0.1% SDS for example for analysis of repetitive regions as described hererin.

The specific sequences, referred to in the present invention, also include sequences which are “functionally equivalent” to said specific sequences. In the present invention functionally equivalent sequences refer to sequences which although not identical to the specific sequences provide the same or substantially the same function. DNA sequences that are functionally equivalent include any substitution, deletion or addition within the sequence. With reference to the present invention functionally equivalent sequences will preferably direct the expression of an exogenous gene constitutively.

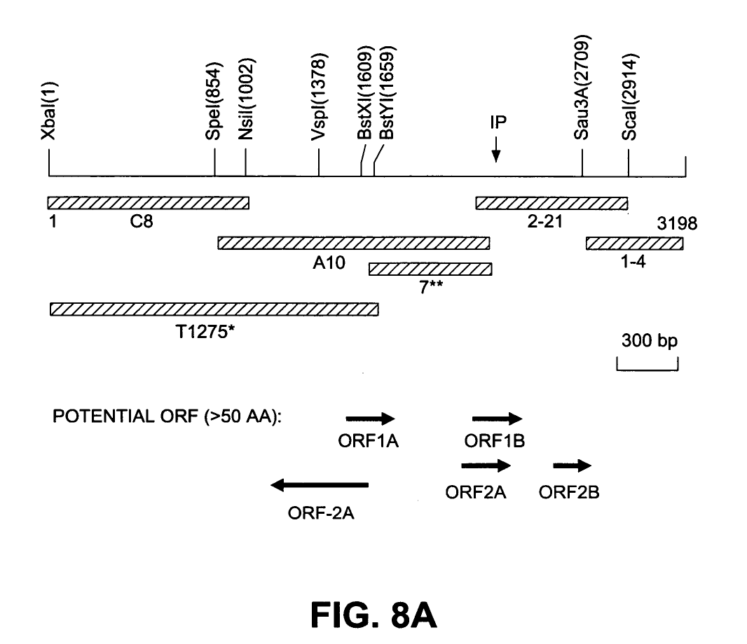

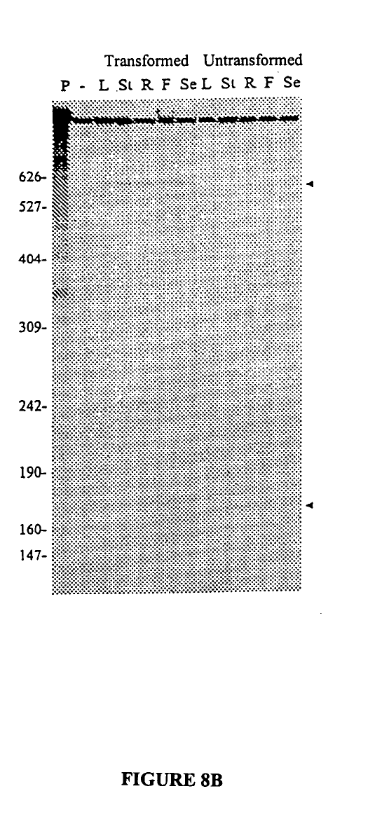

The results presented in the examples indicate that the constitutive expression of GUS activity in the plant T1275 is regulated by a cryptic regulatory element. Similarly, other experiments indicate that homologs of the cryptic regulatory element (for example members of the RENT family) are also effective in obtaining constitutive expression of a coding region of interest under their control. RNase protection assays performed on the region spanning the regulatory element and downstream region did not reveal a transcript for the sense strand (see FIG. 8, Table 2). RNase protection assays were performed using RNA from organs of untransformed tobacco and probes that spanned the T1275 sequence from about −2055 bp to +1200 bp relative to the transcriptional start site. In all tissues tested (leaf, stem, root, flower bud, petal, ovary and developing seed) protected fragments were not detected, in the sense orientation relative to the GUS coding region, with all probes (FIG. 8), and indicates that the site was the same in each organ. Furthermore, GenBank searches revealed no significant sequence similarity with the T1275 sequence. An amino acid identity of about 66% with two open reading frames on the antisense strand of the genomic sequence of T1275 (between about −1418 and −1308; nucleotides 636-746 of SEQ ID NO:1; and between about −541 and −395; nucleotides 1513-1659 of SEQ ID NO:1 relative to the transcriptional start) and an open reading frame of a partial Arabidopsis expressed sequence (GenBank Accession No. W43439) was identified. The sequence which lies downstream of sequences at the T-DNA insertion point in untransformed tobacco shows no significant similarity in GenBank searches. These data suggest that this region is silent in untransformed plants and that the insertion of the T-DNA activated a cryptic regulatory element.

Similar RNase protection assays using probes from tCUP (TI275) against members of the RENT family of sequences (SEQ ID NO's: 5 to 9) indicates that these sequences are also silent in untransformed plants.

Southern analysis indicates that the 2.2 kb regulatory region of T1275 does not hybridize with DNA isolated from soybean, potato, sunflower, Arabidopsis, B. napus, B. oleracea, corn, wheat or black spruce. However, transient assays indicate that this regulatory region can direct expression of the GUS coding region in all plant species tested including canola, tobacco, soybean, alfalfa, potato, Ginseng, peach, pea, Arabidopsis, B. napus, white spruce, corn, wheat, oat and barley (Table 3), indicating that this regulatory element is useful for directing gene expression in both dicot and monocot plants. A fragment of the T1275 (tCUP) regulatory region that exhibits substantial homology with a segment of the RENT family of repetitive elements, and the corresponding fragments from the RENT nucleotide sequences, for example, but not limited to SEQ ID NO's: 5 to 9, and 21 are also active in other species, for example but not limited to pea and Arabidopsis (see FIG. 14).

The following fragments of the members of the RENT family (see SEQ ID NO:21), and there corresponding fragments of SEQ ID NO: 1, have been characterized, and their utility demonstrated in the present invention. For example, the fragment comprising nucleotides from SEQ ID NO: 1 or 22 of:
1724-2224 and nucleotide sequences that are characterized as having greater than 75% sequence identify with nucleotides 1724-2224 of SEQ ID NO: 1 or 22 (see FIG. 13C) exhibit regulatory element activity (e.g. FIG. 14(A));
1875-2086 (DraI-NdeI fragment; core promoter element), see FIG. 5C and Table 6 (−197-GUS-nos);
1875-1992 (DraI −62 fragment)—this fragment enhances expression of the −46 minimal promoter of 35S, and a fragment of T1275, as shown in FIG. 5D (see Dra1-GUS; Dra2-GUS; Dra1-35S; Dra2-35S), and FIGS. 5E-G (Dra1-35S; Dra2-35S), and functions as a transcriptional enhancer;
2084-2224 (NdeI-SmaI fragment, or “N”; Tables 10-12, FIG. 5B (+30-GUS-nos), FIG. 7 (T1275-GUS-nos; 35S-GUS-nos), and FIG. 11 (35S+N-GUS-nos) a translational enhancer; and
2091-2170 (ΔN) see Tables 10-12; a translational enhancer.

The transcriptional start site of T1275 (tCUP) was delimited by RNase protection assay to a single position about 220 bp upstream of the translational initiation codon of the GUS coding region in the T-DNA. The sequence around the transcriptional start site exhibits similarity with sequences favored at the transcriptional start site compiled from available dicot plant genes (T/A T/C A₊₁ A C/A C/A A/C/T A A A/T). Sequence similarity is not detected about 30 bp upstream of the transcriptional start site with the TATA-box consensus compiled from available dicot plant genes (C T A T A A/T A T/A A).

Deletions in the upstream region indicate that negative regulatory elements and enhancer sequences exist within the full length regulatory region. For example deletion of the 5′ region to BstYI (−394 relative to the transcriptional start site; position 1660 of SEQ ID NO: 1 or 22) resulted in a 3 to 8 fold increase in expression of the gene associated therewith (see Table 6 in Examples, and FIG. 5 (C)), indicating the occurrence of at least one negative regulatory element within the XbaI-BstYI portion of the full length regulatory element. Other negative regulatory elements also exist within the XbaI-BstYI fragment of T1275 as removal of an XbaI-PstI fragment also resulted in increased activity (−1304-GUS-nos; Table 6, Examples, and FIG. 5, comprising a deletion of nucleotides 1-1040 of SEQ ID NO: 1 or 22).

An enhancer is also localized within the BstYI-DraI fragment of tCUP as removal of this region results in a 4 fold loss in activity of the remaining regulatory region (−197-GUS-nos; Table 6, Examples, and FIG. 5, comprising a deletion of nucleotides 1-1875 of SEQ ID NO:1 or 22). In addition to the −197 to −62 region (corresponding to nucleotides 1875 to 1992 of SEQ ID NO: 1) exhibiting enhancer-like properties, the region spanning −394 to −62 (corresponding to nucleotides 1660 to 1992 of SEQ ID NO:1) also exhibit similar properties. When the −197 to −62 (nucleotides 1875-1992 of SEQ ID NO:1 or 22) and −394 to −62 (nucleotides 1660-1992 of SEQ ID NO:1 or 22) fragments of T1275 construct are fused with the −46 minimal promoter of 35S, the promoter activities were enhanced to about 150 fold (Dra1-35S FIG. 5(D)). Duplication of the −197 to −62 (Dra2-GUS; FIG. 5(D)), or the −394 to −197 (labelled as Bst1 in FIG. 5(D)) fragments, or a combination of these two fragments, resulted increased regulatory element activity when placed in association with a regulatory element fragment, for example, T1275 (Bst2-GUS; FIG. 5(D)) or 35S (Bst2-35S).

5′ deletions of the regulatory element (see FIGS. 5(A) and (B) and analysis by transient expression using biolistics showed that the regulatory element was active within a fragment 62 bp from the transcriptional start site indicating that the core promoter has a basal level of expression (see Table 5, Examples; and FIGS. 5(H) and (I)). Deletion of a fragment containing the transcriptional start site (see—62(-tsr)-GUS-nos in FIGS. 5(B), (H) and (I); Table 5, Examples) reduced expression dramatically in transgenic tomato, however deletions to +30 did eliminate expression indicating that the region defined from about −12 to about +30 bp contained the core promoter. Deletion of sequences surrounding the transcriptional start site, reduced activity to about 2% of the activity associated with the −62-GUS construct, indicating that the transcriptional start site sequence is required for tCUP regulatory element activity. DNA sequence searches did not reveal conventional core promoter motifs found in plant genes such as the TATA box.

Substitution of nucleotides at −30 to −24, of −62-GUS-nos, with the TATA-box sequence TATATAA (FIGS. 5(D) and (H), increased core promoter activity about 3 fold (FIG. 5(I). Addition of a GCC-box sequence (Hart et al.,1993; Ohme-Takagi and Shinshi, 1995) to −62-GUS-nos resulted in about a four fold increase in activity (see FIG. 5(I)). The results presented in FIGS. 5(D) and (I) demonstrate that the regulatory elements of the present invention may be modulated through a variety of modifications including duplication of fragments that exhibit enhancer or silencer activity, or by substituting, inserting, or adding regulatory elements to enhance or silence tCUP regulatory element activity.

A number of the 5′ regulatory element deletion clones (FIG. 5(C)) were transferred into tobacco by Agrobacterium-mediated transformation using the vector pRD400. Analysis of GUS specific activity in leaves of transgenic plants (see Table 6, Examples) confirmed the transient expression data down to the −197 fragment (nucleotides 1857-2224 of SEQ ID NO: 1). Histochemical analysis of tobacco organs sampled from the transgenic plants indicated GUS expression in leaf, seeds and flowers. Histochemical analysis of Arabidopsis organs revealed GUS activity in leaf, stem flowers and silques when the promoter was deleted to the −394 and −197 fragments (see FIGS. 5(E) to (G)).

As indicated above, a fragment of the regulatory element tCUP (T1275) exhibits substantial homology with a large family of repetitive elements within N. tabacum. These homologous sequences (SEQ ID NO's: 5 to 9; RENT 1, 2, 3, 5 and 7) also exhibit regulatory activity as determined by an increase in the expression of GUS in pea protoplast assays (FIG. 14(A)). This region (−394 tCUP-GUS) was also found to drive the constitutive expression of a coding region of interest in transgenic Arabidopsis (FIG. 14(B)). Therefore, the present invention also describes the regulatory elements associated with members of the RENT family of repetitive elements including tCUP (T1275). The consensus sequence for members of the RENT family is provided in FIGS. 13(C) and 13(D).

Expression of GUS, under the control of T1275 or a fragment thereof, or the modulation of GUS expression arising from T1275 or a fragment thereof, has been observed in a range of species including corn, wheat, barley, oat, tobacco, Brassica, soybean, alfalfa, pea, potato, Ginseng, Arabidopsis, peach, spruce, yeast, fungi, insects and bacterial cells (Table 3, Examples, and FIGS. 14(A), and (B)).

Occurrence of a Post-Transcriptional Regulatory Element in the T1275 Nucleotide Sequence

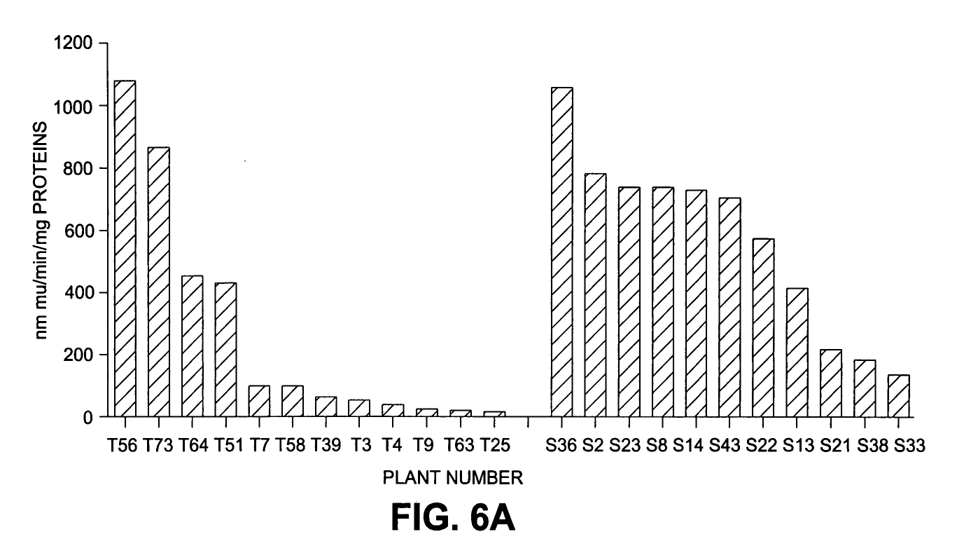

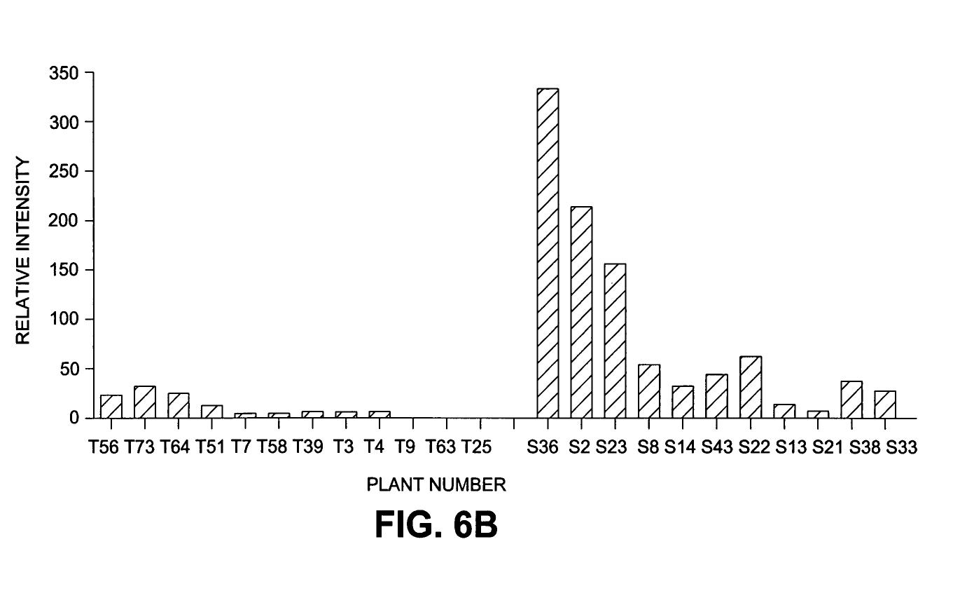

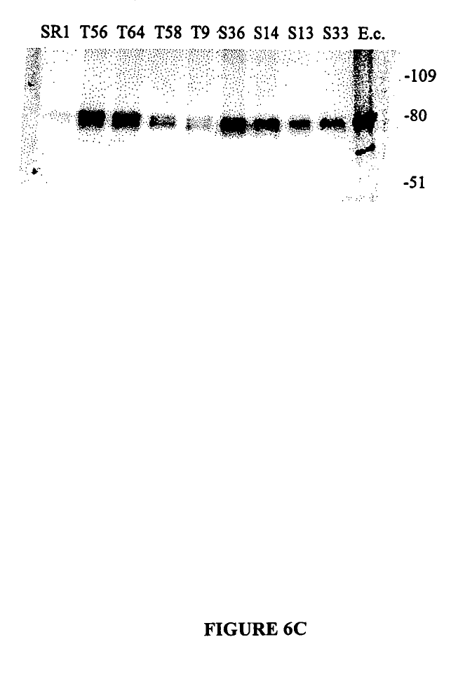

A comparison of GUS specific activities in the leaves of transgenic tobacco SRI transformed with the T1275-GUS-nos gene and the 35S-GUS-nos genes revealed a similar range of values (FIG. 6(A)). Furthermore, the GUS protein levels detected by Western blotting were similar between plants transformed with either gene when the GUS specific activities were similar (FIG. 6(C)). Analysis of GUS mRNA levels by RNase protection however revealed that the levels of mRNA were about 60 fold (mean of 13 measurements) lower in plants transformed with the T1275-GUS-nos gene (FIG. 6(B) suggesting the existence of a post-transcriptional regulatory element in the mRNA leader sequence.

Further analysis confirmed the presence of a regulatory sequence within the NdeI-SmaI fragment of the mRNA leader sequence that had a significant impact on the level of GUS specific activity expressed in all organs tested. Deletion of the NdeI-SmaI fragment (nucleotides 2084-2224 of SEQ ID NO: 1 or 22) from the T1275-GUS-nos gene (FIG. 7) resulted in about a 46-fold reduction in the amount of GUS specific activity that could be detected in leaves of transgenic tobacco cv Delgold (see Table 7). Similar results were also observed in the transgenic tobacco cultivar SR1 and transgenic alfalfa (Table 7). Addition of the same fragment to a 35S-GUS-nos gene construct (FIG. 7) increased the amount of GUS specific activity by about 5-fold in transgenic tobacco and a higher amount in transgenic alfalfa (see Table 7). Increased GUS activity was observed in organs of tobacco and alfalfa plants tranformed with constructs containing NdeI-SmaI fragment (Table 8 and 9). This data is consistent with the presence of a post-transcriptional regulatory element in this fragment.

A modulation of GUS activity was noted in a variety of species that were transformed with a regulatory element of the present invention. For example but not necessarily limited to, the NdeI-SmaI fragment of T1275 (also referred to as “N”) and derivatives or analogues thereof, produced an increase in activity within a variety of organisms tested including a range of plants (Tables 3 and 10, and FIG. 11), white spruce (a conifer; Table 11) and yeast (Table 12).

A shortened fragment of the NdeI-SmaI fragment, (referred to as “ΔN”, “dN”, or “deltaN”) was produced that lacks the out-of frame upstream ATG at nucleotides 2087-2089 of SEQ ID NO: 1 (see FIGS. 10(A) and (B)). Constructs comprising T1275(ΔN)-GUS-nos yielded 5 fold greater levels of GUS activity in leaves of transgenic tobacco compared to plants expressing T1275-GUS-nos. Furthermore, in corn callus and yeast, ΔN significantly increased GUS expression driven by the 35 S promoter (FIG. 119 and Table 10).

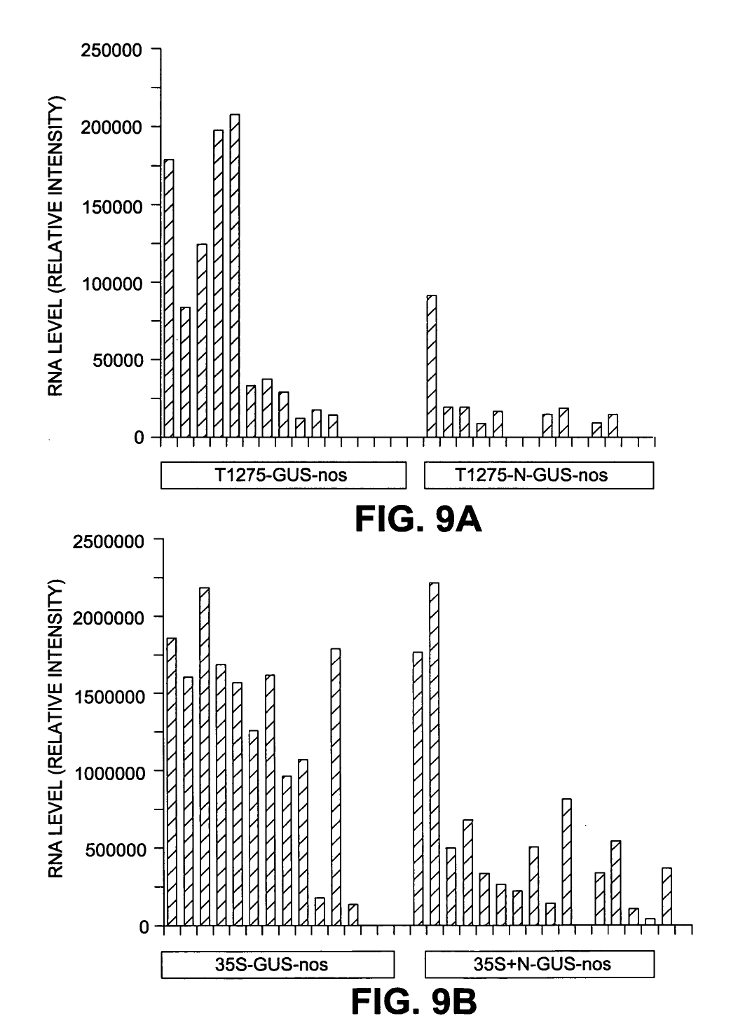

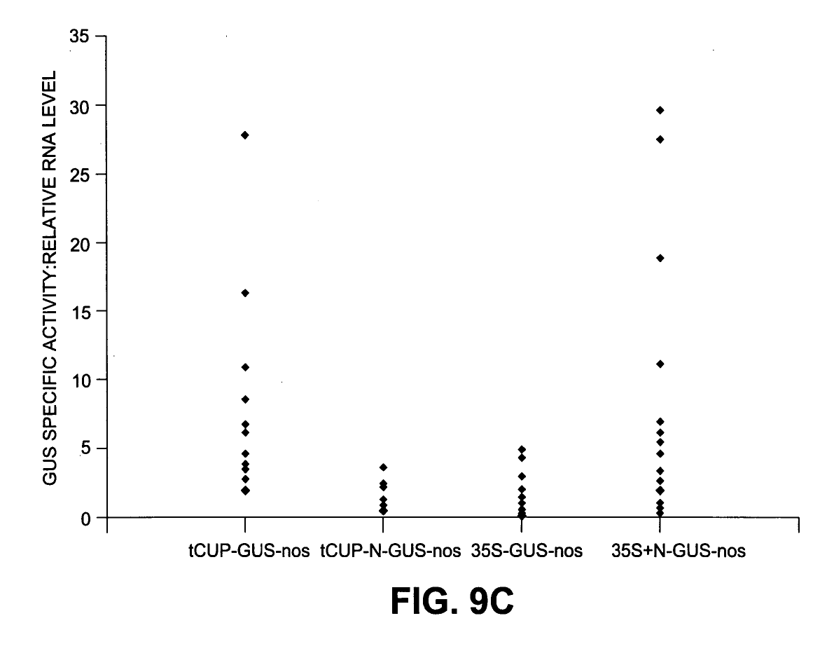

The NdeI-SmaI regulatory elements situated downstream of the transcriptional start site functions both at a transcriptional, and post-transcriptional level. The levels of mRNA examined from transgenic tobacco plants transformed with either T1275-GUS-nos, T1275-N-GUS-nos, 35S-GUS-nos, or 35S+N-GUS-nos, are higher in transgenic plants comprising the NdeI-SmaI fragment under the control of the T1275 regulatory element but lower in those under control of the 35S promoter, than in plants comprising constructs that lack this region (FIGS. 9(A) and (B)). This indicates that this region functions by either modulating transcriptional rates, or the stability of the transcript, or both.

The NdeI-SmaI region also functions post-transcriptionally. The ratio of GUS specific activity to relative RNA level in individual transgenic tobacco plants that lack the NdeI-SmaI fragment is lower, and when averaged indicates an eight fold reduction in GUS activity per RNA, than in plants comprising this region (FIG. 9(C)). Similarly, an increase, by an average of six fold, in GUS specific activity is observed when the NdeI-SmaI region is added within the 35S untranslated region (FIG. 9(C)). The GUS specific activity:relative RNA levels are similar in constructs containing the NdeI-SmaI fragment (tCUP-GUS-nos and 35S+N-GUS-nos). These results indicate that the NdeI-SmaI fragment (nucleotides 2084-2224 of SEQ ID NO: 1 or 22) modulates gene expression post-transcriptionally. Further experiments suggest that this region is a novel translational enhancer. Translation of transcripts in vitro demonstrate an increase in translational efficiency of RNA containing the NdeI to SmaI fragment (see Table 13). Furthermore, the levels of protein produced using mRNAs comprising the NdeI-SmaI fragment are greater than those produced using the known translational enhancer of Alfalfa Mosaic Virus RNA4. These results indicate that this region functions post-transcriptionally, as a translational enhancer.

One or more of the constitutive regulatory elements described herein may be used to drive the expression within all organs or tissues, or both of a plant of a coding region of interest, and such uses are well established in the literature. For example, fragments of specific elements within the 35S CaMV promoter have been duplicated or combined with other regulatory element fragments to produce chimeric regulatory elements with desired properties (e.g. U.S. Pat. No. 5,491,288; U.S. Pat. No. 5,424,200; U.S. Pat. No. 5,322,938; U.S. Pat. No. 5,196,525; U.S. Pat. No. 5,164,316). As indicated above, the constitutive regulatory element or a fragment thereof, as defined herein, may also be used along with other regulatory element, enhancer elements, or fragments thereof, translational enhancer elements or fragments thereof in order to control gene expression. Furthermore, oligonucleotides of 18 bps or longer are useful as probes, for example to identify other members of the RENT family of repetitive sequences, or as PCR primers in identifying or amplifying related DNA or RNA sequences in other tissues or organisms.

Thus this invention is directed to a constitutive regulatory element, associated regulatory elements identified within the tCUP nucleotide sequence (SEQ ID NO: 1 or 22), and combinations comprising one or more than one of these regulatory elements. Further this invention is directed to such regulatory elements and combinations thereof, in a cloning vector, wherein the coding region of interest is under the control of the regulatory element and is capable of being expressed in a plant cell transformed with the vector. This invention further relates to transformed plant cells, transgenic plants regenerated from such plant cells, and seeds produced from these plants. The regulatory element, and regulatory element-gene combination of the present invention can be used to transform any plant cell for the production of any transgenic plant. The present invention is not limited to any plant species.

Therefore, the regulatory elements of the present invention may be used to control the expression of a coding region of interest within desired host expression system, for example, but not limited to:
plants, both monocots and dicots, for example, corn, tobacco, Brassica, soybean, pea, alfalfa, potato, ginseng, wheat, oat, barley, Arabidopsis;
trees, for example peach, spruce;
yeast, fungi, insects, and bacteria.

Furthermore, the regulatory elements as described herein may be used in conjunction with other regulatory elements, such as tissue specific, inducible or constitutive promoters, enhancers, or fragments thereof, and the like. For example, the regulatory region or a fragment thereof as defined herein may be used to regulate gene expression of a coding region of interest spatially and developmentally within a plant of interest or within a heterologous expression system, for example yeast, insects, or fungi expression systems. Regulatory regions or fragments thereof, including enhancer fragments of the present invention, may be operatively associated with a heterologous nucleotide sequence including heterologous regulatory regions to increase, decrease, or otherwise modulate, the expression of a coding region of interest within a host organism. A coding region of interest may include, but is not limited to, a gene that encodes a pharmaceutically active protein, for example growth factors, growth regulators, antibodies, antigens, their derivatives useful for immunization or vaccination and the like. Such proteins include, but are not limited to, interleukins, insulin, G-CSF, GM-CSF, hPG-CSF, M-CSF or combinations thereof, interferons, for example, interferon-α, interferon-β, interferon-τ, blood clotting factors, for example, Factor VIII, Factor IX, or tPA or combinations thereof. A coding region of interest may also encode an industrial enzyme, protein supplement, nutraceutical, or a value-added product for feed, food, or both feed and food use. Examples of such proteins include, but are not limited to proteases, oxidases, phytases chitinases, invertases, lipases, cellulases, xylanases, enzymes involved in oil metabolic and biosynthetic pathways etc. A coding region of interest may also encode a protein imparting or enhancing herbicide resistance or insect resistance of a plant transformed with a construct comprising a constitutive regulatory element as described herein.

A list of the nucleotide sequences provided in the present invention is provided in Table 1b.

TABLE 1b
Nucleotide Sequence Summary

| SEQ ID NO: | Name of sequence |
|---|---|
| 1 | T1275 (tCUP) |
| 2 | Nde-Sma |
| 3 | ΔN |
| 4 | ΔN^{m} |
| 5 | RENT 1 |
| 6 | RENT 2 |
| 7 | RENT 3 |
| 8 | RENT 5 |
| 9 | RENT 7 |
| 10 | pr-1S (primer) |
| 11 | pr-3A (primer) |
| 12 | pr-2S (primer) |
| 13 | pr-4S (primer) |
| 14 | pr-5A (primer) |
| 15 | pr-6S (primer) |
| 16 | pr-7S (primer) |
| 17 | pr-8A (primer) |
| 18 | GCC-62-GUS fragment |
| 19 | HindIII primer |
| 20 | BglII primer |
| 21 | RENT consensus sequence |
| 22 | tCUP consensus sequence |

The present invention will be further illustrated in the following examples.

### EXAMPLES

### Characterization of a Constitutive Regulatory Element—GUS Fusion

Transfer of binary constructs to Agrobacterium and leaf disc transformation of N. tabacum SR1 were performed as described by Fobert et al. (1991, Plant Mol. Biol. 17, 837-851). Plant tissue was maintained on 100 μg/ml kanamycin sulfate (Sigma) throughout in vitro culture.

From the transgenic plants produced, one of these, T1275, was chosen for detailed study because of its high level and constitutive expression of GUS.

Fluorogenic and histological GUS assays were performed according to Jefferson (Plant Mol. Biol. Rep., 1987, 5, 387-405), as modified by Fobert et al. (Plant Mol. Biol., 1991, 17, 837-851). For initial screening, leaves were harvested from in vitro grown plantlets. Later nine different tissues: leaf (L), stem (S), root (R), anther (A), petal (P), ovary (O), sepal (Se), seeds 10 days post anthesis (S1) and seeds 20 days post-anthesis (S2), were collected from plants grown in the greenhouse and analyzed. For detailed, quantitative analysis of GUS activity, leaf, stem and root tissues were collected from kanamycin resistant F1 progeny grown in vitro. Floral tissues were harvested at developmental stages 8-10 (Koltunow et al., 1990, Plant Cell 2, 1201-1224) from the original transgenic plants. Flowers were also tagged and developing seeds were collected from capsules at 10 and 20 dpa. In all cases, tissue was weighed, immediately frozen in liquid nitrogen, and stored at −80° C.

Tissues analyzed by histological assay were at the same developmental stages as those listed above. Different hand-cut sections were analyzed for each organ. For each plant, histological assays were performed on at least two different occasions to ensure reproducibility. Except for floral organs, all tissues were assayed in phosphate buffer according to Jefferson (1987, Plant Mol. Biol. Rep. 5, 387-405), with 1 mM X-Gluc (Sigma) as substrate. Flowers were assayed in the same buffer containing 20% (v/v) methanol (Kosugi et al., 1990, Plant Sci. 70, 133-140).

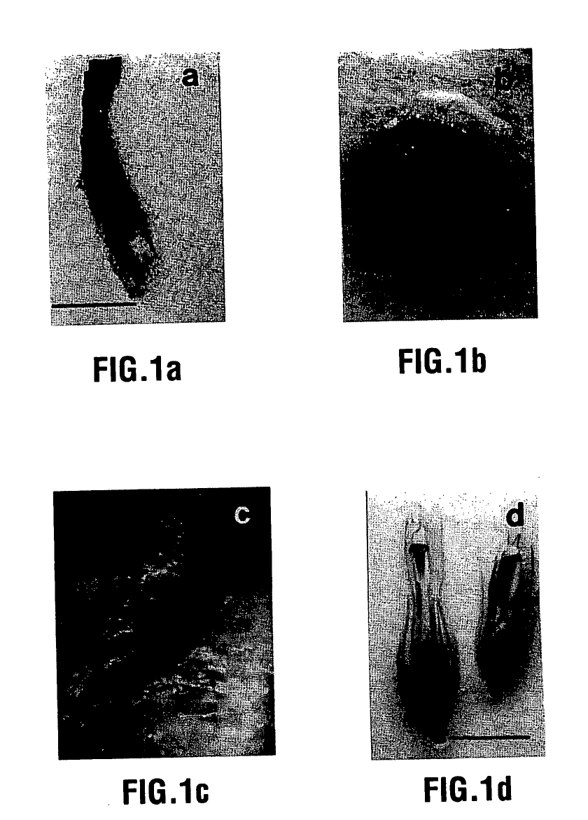

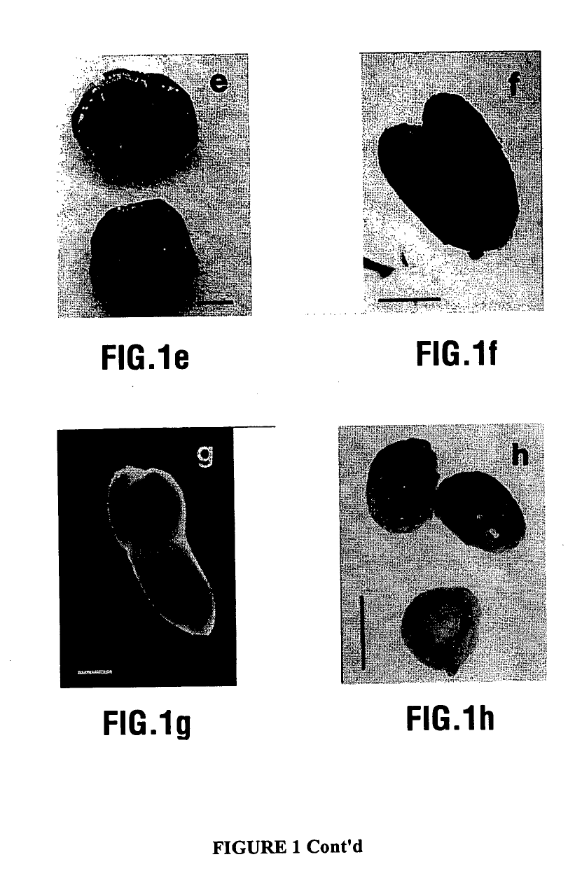

GUS activity in plant T1275 was found in all tissues. FIG. 1 shows the constitutive expression of GUS by histochemical staining with X-Gluc of T1275, including leaf (a), stem (b), root (c), flower (d), ovary (e), embryos (f and g), and seed (h).

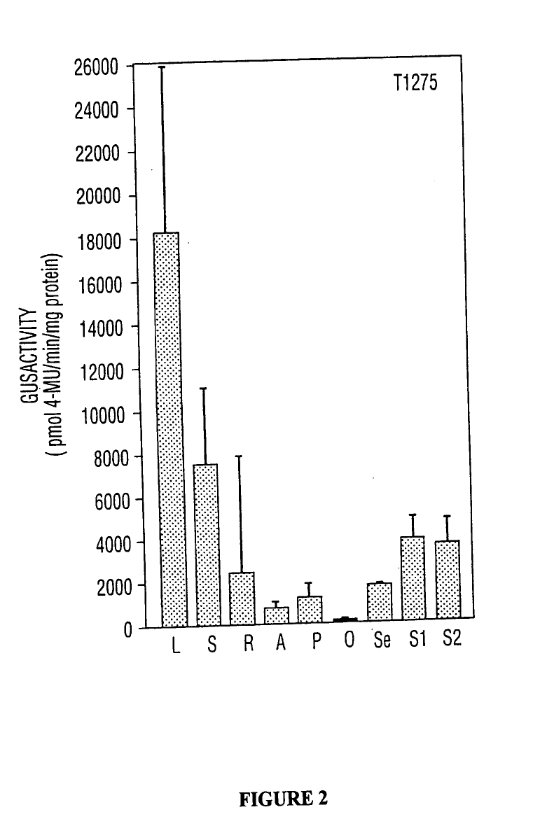

Constitutive GUS expression was confirmed with the more sensitive fluorogenic assay of plant tissue from transformed plant T1275. These results are shown in FIG. 2. GUS expression was evident in all tissue types including leaf (L), stem (S), root (R), anther (A), pistil (P), ovary (O), sepal (Se), seeds at 10 dpa (S1) and 20 dpa (S2). Furthermore, the level of GUS expression is comparable to the level of expression in transformed plants containing the constitutive promoter CaMV 35S in a GUS-nos fusion. As reported by Fobert et al. (1991, Plant Molecular Biology, 17: 837-851) GUS activity in transformed plants containing pBI121 (Clontech), which contains a CaMV 35S-GUS-nos chimeric gene, was as high as 18,770±2450 (pmole MU per minute per mg protein).

Genetic Analysis of Transgenic Plant T1275

The T-DNA contains a kanamycin resistance gene. Seeds from self-pollinated transgenic plants were surface-sterilized in 70% ethanol for 1 min and in undiluted Javex bleach (6% sodium hypochloride) for 25 min. Seeds were then washed several times with sterile distilled water, dried under laminar flow, and placed in Petri dishes containing MS0 medium supplemented with 100 μg/ml kanamycin as described in Miki et al. (1993, Methods in Plant Molecular Biology and Biotechnology, Eds., B. R. Glick and J. E. Tompson, CRC Press, Boca Raton, 67-88). At least 90 plantlets were counted for each transformant. The number of green (kanamycin-resistant) and bleached (kanamycin-sensitive) plantlets were counted after 4-6 weeks, and analyzed using the Chi² test at a significance level of P<0.05.

The genetic analysis results are shown below in Table 1c, which demonstrates that the T-DNA loci segregated as a single locus of insertion.

TABLE 1c
Genetic Analysis of Transgenic Plant T1275

| No. of | No. of | | | |
|---|---|---|---|---|
| Progeny | Progeny | Observed | Expected | |
| Km^{r} | Km^{s} | Ratio | Ratio | Chi² |
| 262 | 88 | 3:1* | 3:1 | 0 |

*Consistent with a single dominant gene

Southern Blot Analysis

The T-DNA in the transgenic plant T1275 was analyzed using either a GUS gene coding region probe or a nptII gene coding region probe.

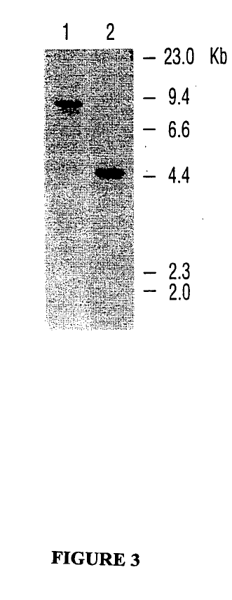

Genomic DNA was isolated from freeze-dried leaves using the protocol of Sanders et al. (1987, Nucleic Acid Res. 15, 1543-1558). Ten micrograms of T1275 DNA was digested for several hours with EcoRI using the appropriate manufacturer-supplied buffer supplemented with 2.5 mM spermidine. After electrophoresis through a 0.8% TAE agarose gel, Southern blot analysis was conducted using standard protocols. As the T-DNA from the construct containing the constitutive regulatory element—GUS-nos construct contains only a single Eco RI recognition site the hybridizing fragments are composed of both T-DNA and flanking tobacco DNA sequences. The length of the fragment will vary depending on the location of the nearest Eco RI site. Using the GUS gene as a probe (FIG. 3—lane 1), the fragment to the nearest Eco RI site in the plant DNA will be detected. With T1275, one such fragment was located. Using the nptII coding region as a probe (FIG. 3—lane 2), which hybridizes to sequences on the opposite side of the Eco RI site, again only one hybridization band was evident. As can also be seen in FIG. 3, no major rearrangements occurred within the T-DNA.

Cloning and Analysis of the Constitutive Regulatory element—GUS Fusion

Genomic DNA was isolated from leaves according to Hattori et al. (1987, Anal. Biochem. 165, 70-74). Ten μg of T1275 total DNA was digested with EcoRI and XbaI according to the manufacturer's instructions. The digested DNA was size-fractionated on a 0.7% agarose gel. The DNA fragments of about 4 to 6 kb were isolated from the gel using the Elu-Quick kit (Schleicher and Schuell) and ligated to lambdaGEM-2 arms previously digested with EcoRI and XbaI and phosphatase-treated. About 40,000 plaques were transferred to a nylon membrane (Hybond, Amersham) and screened with the ³²P-labelled 2 kb GUS insert isolated form pBI121, essentially as described in Rutledge et al. (1991, Mol. Gen Genet. 229, 31-40). The positive clones were isolated. The XbaI-EcoRI fragment (see restriction map FIG. 4) was isolated from the lambda phage and cloned into pTZ19R previously digested with XbaI and EcoRI and treated with intestinal calf phosphatase.

The plant DNA sequence within the clone SEQID NO: 1 has not been previously reported in sequence data bases. It is not observed among diverse species as Southern blots did not reveal bands hybridizing with the fragment in soybean, potato, sunflower, Arabidopsis, B. napus, B. oleracea, corn, wheat or black spruce (data not shown). In tobacco, Southern blots did not reveal evidence for gross rearrangements at or upstream of the T-DNA insertion site (data not shown).

The T1275 Regulatory Element is Cryptic

The 4.2 kb fragment containing about 2.2 kb of the T1275 regulatory element fused to the GUS gene and the nos 3′ was isolated by digesting pTZ-T1275 with HindIII and EcoRI. The isolated fragment was ligated into the pRD400 vector (Datla et al., 1992, Gene, 211:383-384) previously digested with HindIII and EcoRI and treated with calf intestinal phosphatase. Transfer of the binary vector to Agrobacterium tumefaciens and leaf disc transformation of N. tabacum SR1 were performed as described above. GUS activity was examined in several organs of many independent transgenic lines. GUS mRNA was also examined in the same organ by RNase protection assay (Melton et al, 1984, Nucleic Acids Res. 121: 7035-7056) using a probe that mapped the mRNA 5′ end in both untransformed and transgenic tissues. RNA was isolated from frozen-ground tissues using the TRIZOL Reagent (Life Technologies) as described by the manufacturer. For each assay 10-30 ug of total RNA was hybridized to an antisense RNA probe as described in FIG. 8(A). Assays were performed using the RPAII kit (Ambion CA) as described by the manufacturer. The protected fragments were separated on a 5% Long Ranger acrylamide (J. J. Baker, N.J.) denaturing gel which was dried and exposed to Kodak X-RP film.

RNase protection assays performed with RNA from leaves, stem, root, developing seeds and flowers of transgenic tobacco revealed a single protected fragment in all organs indicating a single transcription start site that was the same in each organ, whereas RNA from untransformed tobacco tissues did not reveal a protected fragment (FIG. 8(B)). The insertion site, including 1200 bp downstream, was cloned from untransformed tobacco as a PCR fragment and sequenced. A composite restriction map of the insertion site was assembled as shown in FIG. 8(A). RNA probes were prepared that spanned the entire region as shown in FIG. 8(A). RNase protection assays did not reveal transcripts from the sense strand as summarized in Table 2. These data suggest that the insertion site is transcriptionally silent in untransformed tobacco and is activated by T-DNA insertion. The region upstream of the insertion site is therefore another example of a plant cryptic regulatory element.

TABLE 2
Summary of the RNase Protection Assays of the insertion site in
untransformed tobacco. See FIG. 8 (A) for probe positions.

| Probe | Rnase Protection Assay result |
|---|---|
| Looking for “sense” RNAs | (relative to the T1275 regulatory element) |
| C8-EcoRI | many bands, all in tRNA (negative control) |
| A10-HindIII | no bands |
| 2-21-HindIII | no bands |
| 1-4 SmaI | many bands, all in tRNA |
| 7-EcoRI | faint bands, all in tRNA |

Constitutive Activity of the T1275 Regulatory Element

For analysis of transient expression of GUS activity mediated by biolistics (Sandford et al, 1983, Methods Enzymol, 217: 483-509), the XbaI-EcoRI fragment was subcloned in pUC19 and GUS activity was detected by staining with X-Gluc as described above. Leaf tissue of greenhouse-grown plants or cell suspension cultures were examined for the number of blue spots that stained. As shown in Table 3, the T1275—GUS nos gene was active in each of the diverse species examined and can direct expression of a coding region of interest in all plant species tested. Leaf tissue of canola, tobacco, soybean, alfalfa, pea, Arabidopsis, potato, Ginseng, peach, and cell suspensions of oat, corn, wheat, barley and white spruce exhibited GUS-positive blue spots after transient bombardment-mediated assays and histochemical GUS activity staining. This suggests that the T1275 regulatory element may be useful for directing gene expression in both dicot and monocot plants.

TABLE 3
Transient Expression of GUS Activity in Tissues of Diverse Plant
Species

| Tissue Source | Species | GUS Activity* |
|---|---|---|
| Leaf | Soybean | +++ |
| | Alfalfa | ++ |
| | Arabidopsis | + |
| | Potato | ++ |
| | Ginseng | ++ |
| | Peach | + |
| Leaf disc | Tobacco | ++ |
| | B. napus | + |
| | Pea | + |
| Cell Cultures | Oat | + |
| | Corn | + |
| | Wheat | + |
| | Barley | ++ |
| | White spruce | ++ |

*Numbers of blue spots: 1-10 (+), 10-100 (++), 100-400 (+++)

For analysis of GUS expression in different organs, lines derived from progeny of the above lines were examined in detail. Table 4 shows the GUS specific activities in one of these plants. It is expressed in leaf, stem, root, developing seeds and the floral organs, sepals, petals, anthers, pistils and ovaries at varying levels, confirming constitutive expression. Introduction of the same vector into B. napus also revealed expression of GUS activity in these organs (data not shown) indicating that constitutive expression was not specific to tobacco. Examination of GUS mRNA in the tobacco organs showed that the transcription start sites were similar (FIG. 8(B)), and the level of mRNA was similar except in flower buds where it was lower (Table 4).

TABLE 4
GUS Specific Activity and Relative RNA Levels in the Organs of
Progeny of Transgenic Line T64

| | Relative GUS RNA | GUS Specific Activity | |
|---|---|---|---|
| | Levels in T64 | (picomol/MU/min/mg protein) | |
| | Progeny (grey scale | Transformed | Untransformed |
| Organ | units) | Tobacco T64 | Tobacco |
| Leaf | 1774 | 988.32 | 3.02 |
| Stem | 1820 | 826.48 | 7.58 |
| Root | 1636 | 4078.45 | 22.18 |
| 14 day post | 1790 | 253.21 | 10.03 |
| anthesis Seeds | | | |
| Flower - buds | 715 | 2.59 | ND* |
| Petals | ND* | 28.24 | 1.29 |
| Anthers | ND* | 4.64 | 0.35 |
| Pistils | ND* | 9.76 | 1.72 |
| Sepals | ND* | 110.02 | 2.48 |
| Ovary | ND* | 4.42 | 2.71 |

*Not Done

T1275 Sequence Comparison

The present invention provides an isolated nucleotide sequence selected from the group consisting of SEQ ID NO: 1, and a nucleotide sequence that hybridizes to SEQ ID NO: 1 under a condition selected from the group consisting of:
hybridizing overnight (16-20 hrs) in a solution comprising 7% SDS, 0.5M NaPO4 buffer at pH 7.2, and 10 mM EDTA at 65° C. and washing for one hour at 60° C. in a solution comprising 0.1×SSC and 0.1% SDS;
hybridizing overnight (16-20 hrs) in a solution comprising 7% SDS, 0.5M NaPO4 buffer at pH 7.2, and 10 mM EDTA at 65° C. and washing for one hour at 65° C. in a solution comprising 2×SSC and 0.1% SDS; and
hybridizing overnight (16-20 hrs) in a solution comprising 4×SSC at 65° C. and washing for one hour in 0.1×SSC at 65° C.;
   wherein the nucleotide sequence confers constitutive expression of a coding region of interest linked in operative association therewith.

The Tₘ of T1275 is compared to the closest homologue identified in a sequence similarity search, an Arabidopsis phytochelatin synthase gene (Genbank Accession No. AF085230) that exhibits 52% similarity with T1275. The following analysis indicates that the T1275 sequence and nucleic acid sequences that hybridize to T1275 under stringent conditions defined herein are unique.

The Tₘ° C., under the hybridization conditions stated above for T1275 and AF085230, are provided in table 4A below, where,
% similarity was calculated using NCBI Blast 2 program (available through the NIH at: ncbi.nlm.nih.gov/cgi-bin/BLAST/; parameters for alignments were set at: match 5; mismatch −4; gap open 5; gap extension 2; x_dropoff 50; expect 10; worksize 11 and filter ON).
Conditions for A, B and C listed in Table 4A are:
   A=7%SDS, 0.5M NaPO4, 10 mM EDTA, at 65° C. (hybridization)
   B=2×SSC, 0.1%SDS, at 65° C. (washing)
   C=0.1×SSC, 0.1%SDS, at 60° or 65° C. (washing)
Tₘ (perfect match) is calculated using the formula described in Baldino et al (Baldino, Chesselet and Lewis 1989. High-resolution in situ hybridization histochemistry. Methods in Enzymology 168: 761-777) using the following formula:
   Tm=81.5+16.6(log[Na+])+0.41(%G+C)−675/(PL)−0.65(%formamide).
PL is the probe length in bases;
Tm (heterologous match) was calculated by the method as described in Baldino et al (Baldino, Chesselet and Lewis 1989. High-resolution in situ hybridization histochemistry. Methods in Enzymology 168: 761-777) and Bonner et al (Bonner, Brenner, Neufeld and Britten 1973. Reduction in the rate of DNA reassociation by sequence divergence. J. Mol. Biol. 81: 123-135), using the following formula:
   Tm(heterologous match)=Tm(perfect match)−1.0(%mismatches, including gaps).

TABLE 4A

| | |
|---|---|
| Tₘ ° C. | Tₘ ° C. |

| | | |
|---|---|---|
| nucleotide sequence of % similarity with | (perfect match) | (heterologous match) |

| SEQ ID NO:1 | AF085230 | A | B | C | A | B | C |
|---|---|---|---|---|---|---|---|
| 1-2224 | 52 | 79 | 73 | 53 | 31 | 25 | 5 |

These results of the above calculations show that there is about 1 degree C. of decrease in Tm for each % mismatch between two DNA sequences. Assuming a perfect match (100% similarity, which is not the case) between the sequence disclosed in AF085230 and that of SEQ ID NO: 1, the results shown in Table 4A demonstrate that a Tₘ of less than 79°, 73°, and 53° C. is required to detect hybridization between nucleotides 1-2224 of SEQ ID NO: 1 and the sequence of AF085230 under the hybridization and washing conditions stated above. Furthermore, taking into account the % similarity between nucleotides 1-2224 of SEQ ID NO: 1 and the sequence of AF085230, the results in Table 4A demonstrate that a hybridization temperature of greater than 31 ° C. (Tₘ heterologous match) will not result in hybridization between nucleotides 1-2224 of SEQ ID NO: 1 and AF085230.

As the temperatures stated for hybridization above are from 60° to 65° C., and are well above the calculated Tm's indicated in Table 4A, above, the hybridization conditions stated for T1275 do not detect the nucleotide sequence comprising AF085230. Therefore, the T1275 sequence and nucleic acid sequences that hybridize to T1275 under stringent conditions defined herein are unique.

Identification of Regulatory Elements within the Full Length T1275 Regulatory Element

An array of deletions of the full length regulatory region of T1275 were prepared, as identified in FIGS. 5(A) and (B), for further analysis of the cryptic regulatory element.

Plasmid Construction

Deletion and replacement constructs were created in the vector pBI221 (Clontech), which contains the GUS (uidA) coding region driven by the CaMV 35S promoter and the NOS terminator. Independent constructs representing 5′ deletions of the tCUP were generated at convenient restriction sites within the tCUP sequence. The CaMV 35S promoter of pBI221 was replaced with the deletion fragments of tCUP to generate −1304-GUS, −684-GUS, −394-GUS, −197-GUS and −62-GUS. The numbers represent the nucleotide numbers relative to the transcription initiation site.

Fragments to test the enhancer elements between the fragments −394 to −62 (1660-1992 of SEQ ID NO:1 or 22) and −197 to −62 (1875-1992 of SEQ ID NO:1 or 22) relative to the transcription start site of the tCUP were amplified by PCR with Taq DNA polymerase. The fragment from −394 to −62 was amplified with pr-1 and pr-3 primers:

| | | |
|---|---|---|
| pr-1 S: | TTGCCTGCAGGGGATCTTCTGCAAGCATC; | (SEQ ID NO:10) |
| and | | |
| pr-3 A: | TCAAATGCATGGATCAAAAGGGGAAAC, | (SEQ ID NO:11) |

and the fragment from −197 to −62 was amplified with pr-2
pr-2 S: GGAGCTGCAGGCTATTTAAATACTAGCC (SEQ ID NO: 12) and
pr-3 primers. All primers had additional nucleotides at the 5′ ends to give the PstI restriction sites for subcloning PCR products. The PCR products were ligated into the PstI sites located upstream of the −394-GUS and −197-GUS to generate −394(2×)-GUS and −197(2×)-GUS constructs.

A −46 minimal 35S promoter (−46-35S) was generated by PCR using the pr-4 and pr-5 primers:

| | | |
|---|---|---|
| pr-4 S: | CACTCTGCAGGCAAGACCCTTCCTCTATA; | (SEQ ID NO:13) |
| pr-5 A: | ATATAAGCTTTGGGGTTTCTACAGGACG | (SEQ ID NO:14)) |

and pBI221 DNA as a template. The PCR product was digested with PstI and BamHI, and the resulting fragment was used to replace the PstII and BamHI fragment in pBI221. The fragment from −197 to −62 of tCUP (nucleotides 1875-1992 of SEQ ID NO: 1 or 22) was subcloned into the the PstI sites located upstream of the −46-35S-GUS to generate −197-35S-GUS, −197R-35S-GUS and −197(2×)-35S-GUS constructs.

The −12-GUS construct was generated by PCR using the pr-6 and pr-5 primers:

| | | |
|---|---|---|
| pr-5 A: | ATATAAGCTTTGGGGTTTCTACAGGACG; | (SEQ ID NO:14) |
| pr-6 S: | GAGAAGATCTCCAAACACCCCTAACTCTATC. | (SEQ ID NO:15) |

The PCR product was digested with XbaI and KpnI, and the resulting fragment was used to replace the XbaI and KpnI fragment in tCUP-GUS. To generate the −62-tsr-GUS construct, the DNA sequence between −62 and −12 of tCUP was amplified with the pr-7 and pr-8 primers:

| | | |
|---|---|---|
| pr-7 S: | TTGATCATT TTGATCAACGCCCAG; | (SEQ ID NO:16) |
| pr-8 A: | AGGGGGTGCATATGAATTAAAAAAGGAAAAG. | (SEQ ID NO:17) |

The PCR product was digested with XbaI and NdeI, and the resulting fragment was used to replace the XbaI and NdeI fragment in tCUP-GUS. The TA30-GUS construct was generated using pr-9 and pr-5 primers. To generate GCC-62-GUS construct, a 51-bp fragment:

| | | |
|---|---|---|
| GCC-62-GUS fragment: | GCATAAGAGCCGCCACTAAAATAAGACCGATCAAATAAGAGCCGCCATGCA | (SEQ ID NO:18) |

containing two GCC boxes (GCCGCC; Ohme-Takagi and Shinshi, 1995, Ethylene-inducible DNA binding proteins that interact with an ethylene-responsive element. Plant Cell 7: 173-182) was ligated into the PstI site located upstream of the −62-GUS construct.
Plant Transformation and Selection

Arabidopsis thaliana (ecotype Columbia) was grown in a growth chamber (16 hr of light and 8 hr of darkness at 23° C.) after a 2-4 day vernalization period. For growth under sterile conditions, seeds were surface sterilized (15 min incubation in 5% [v/v] sodium hypochlorite, and a three-time rinse in sterile distilled water) and sown on half-strength Murashige and Skoog salts (Sigma) supplemented with 1% sucrose, pH 5.7, and 0.8% (w/v) agar in Petri dishes.

All the consticuts and GUS fusion were subcloned into the pRD400 (Datla R S, Hammerlindl J K, Panchuk B, Pelcher L E, Keller W: Modified binary plant transformation vectors with the wild-type gene encoding NPTII. Gene 211: 383-384, 1992) or pCAMBIA2300 (Cambia, Canberra, Australia) binary vectors for plant transformation. Plant transformation plasmids were electroporated into Agrobacterium tumefaciens GV3101 (Van Larebeke, N, Engler, G, Holsters, M, Van den Elscker, S, Zainen, I, Schilperoort, R A, and Schell, J: Large plasmid in Agrobacterium tumefaciens essential for crown gall-inducing ability. Nature 252,169-170, 1974) as described by Shaw (Shaw C H: Introduction of cloning plasmids into Agrobacterium tumefaciens. Meth Mol Biol 49, 33-37, 1995). The Agrobacterium-mediated transformation of Arabidopsis thaliana was performed as described (Clough S J, Bent A F: Floral dip: a simplified method for Agrobacterium-mediated transformation of Arabidopsis thaliana. Plant J. 16: 735-743, 1998), with the following modifications. Plants with immature floral buds and few siliques were dipped into a solution containing Agrobacterium tumefaciens, 2.3 g/L MS salts (Sigma), 5% (w/v) sucrose and 0.03% Silwet L-77 (Lehle Seeds, Round Rock, Tex.) for 0.5 min. T1 seeds were collected, dried at 25° C., and sown on sterile media containing 40 μg/mL kanamycin to select the transformants. Surviving T1 plantlets were transferred to soil. 15 to 30 independent transgenic lines for each construct were selected and used for the analysis of GUS activity.

Regulatory Element Activity in Tomato: Protoplast Isolation and Electroporation

Young and fully expanded leaves were excised from about 4 weeks old tomato plants and surface sterilized in 5% commercial bleach (Javex) (1% NaOCl). The abaxial surface of leaves were gently rubbed with carborandum powder and rinsed three times with sterile water. After removing midribs, the remaining leaf blades were cut by sharp razor into small pieces and floated on enzyme mixture containing 0.3% Cellulase Onozuka R-10 (Yakult Honsha), 0.15% macerozyme R-10 (Yakult Honsha) and 0.4 M sucrose.

After overnight incubation in dark at 30° C., protoplasts were collected by filtration through a 100 μm nylon mesh filter followed by centrifrigation at 500 rpm for 5 min. The floated protoplasts were gently collected by a wide bore pipette and washed twice with electroporation buffer (150 mM KCl and 0.4 M mannitol) for 5 min at 400 rpm and finally suspended at approximately 1×10⁶/ml in 0.5 M mannitol containing 150 μM MgCl₂.

The viability of protoplasts was confirmed by fluorescin diacetate and alanine blue staining and protoplasts were kept on ice for 30 minutes prior to electroporation. A 25-30 μg plasmid DNA (see FIG. 5(H) for added constructs) was added to 500 μl protoplast syspension, mixed gently and electroporated at 100 μF and 200 Volts using Gene Pulser II (BioRad). To normalize for transfection efficiency, the CaMV 35 S promoter-luciferase plasmid was cotransfected in each experiment. The electroporated protoplasts were kept on ice for 15-30 min, centrifuged for 5 min at 500 rpm and mixed with 0.5 ml Murashige and Skoog medium (containing 3% sucrose, 9% mannitol, 0.1 mM MgSO₄, 2 mg/L naphthylacetic acid and 0.5 mg/L benzyladenine). The cultures were kept in dark at 25° C. for 24 hr, and cells were collected in microcentrifuge tubes. To each 500 μl of protoplast suspension 200 μl of buffer solution (100 mM sodium phosphate, pH 7.8, 1 mM EDTA, 0.5% Triton X-100, 70 mM 2-mercaptoethanol and 10% glycerol) was added and protoplasts were lysed for lucerifase and GUS assay.

Deletion Analysis of tCUP

In order to delineate functional regions of the tCUP regulatory, a series of 5′ deletion constructs were made (FIG. 5(J)), and activities were examined in leaves of transgenic Arabidopsis plants. As shown in FIG. 5(K) and Table 5 below, all sequences from about −2054 to −684 (nucleotides about 290 to 1370 of SEQ ID NO: 1 or 22) relative to the transcription initiation site of the tCUP promoter could be deleted with no significant effect on promoter activity. Deletion of sequences to −394 and −197 (nucleotides 1660-1875 of SEQ ID NO: 1 or 22) decreased expression about 40% and 60%, respectively. The −62 deletion construct reduced GUS activity to a level slightly over background. These results indicated that the −62 fragment contained the minimal promoter and positive cis-regulatory elements were potentially located in the regions from:
−684 to −394 (nucleotides 1370-1660 of SEQ ID NO:1 or 22);
−394 to −197 (nucleotides 1660-1875 of SEQ ID NO:1 or 22); and
−197 to −62 (nucleotides 1875-1992 of SEQ ID NO:1 or 22).
   Identification of Enhancer Elements

To locate enhancer activities within the fragments −394 to −62 (nucleotides 1660-1992 of SEQ ID NO:1 or 22), and −197 to −62 (nucleotides 1875-1992 of SEQ ID NO: 1 or 22), these fragments were duplicated in the promoter constructs, −394(2X)-GUS and −197(2X)-GUS (Figure (J)), and GUS activity was analyzed in transgenic Arabidopsis plants. As shown in FIG. 5(K), insertion of two copies of −197 to −62 and −394 to −62 fragments (nucleotides 1660-1992 and 1875-1992, respectively, of SEQ ID NO: 1 or 22) increased promoter activity about 1.5 to 2-fold compared with the constructs with only one copy of these fragments.

To evaluate whether the enhancers within fragment −197 to −62 (nucleotides 1875-1992 of SEQ ID NO: 1 or 22) could function with other core promoters, the fragment was also fused to the −46 minimal promoter of CaMV 35S (FIG. 5(L)). As shown in FIG. 5(M), insertion of one copy the fragment in both the forward and reverse orientation increased GUS activity by about 15-fold in leaves of transgenic Arabisopsis. Insertion of two copies further enhanced GUS activity by 40-fold. This suggests that the fragment −197 to −62 (nucleotides 1875-1992 of SEQ ID NO: 1 or 22) may function as a transcriptional enhancer element.

Analysis of Core Promoter Region

To analyze the tCUP core promoter, a series of deletions or modifications surrounding the transcriptional start site were made (FIG. 5(H)). Promoter activities were examined using a transient assay in tomato protoplasts (FIG. 5(I)):
deletion of the core promoter to −12 (position 2042 of SEQ IDNO: 1 or 24) decreased GUS activity by 40%;
deletion of the sequence surroundings the transcription start site reduced it to 2% of the −62-GUS construct activity, suggesting that the transcription start site sequence was essential for tCUP promoter activity;
substitution of the sequence −30 to −24 with a TATA-box (TATATAA) in the −62-GUS construct increased the promoter activity about 3-fold;
addition of GCC-box sequences (Hart C M, Nagy F and Meins Jr F: A 61 bp enhancer element of the tobacco beta-1,3-glucanase B gene interacts with one or more regulated nuclear proteins. Plant Mol Biol 21, 121-131,1993; Ohme-Takagi M, Shinshi H: Ethylene-inducible DNA binding proteins that interact with an ethylene-responsive element. Plant Cell 7: 173-182,1995) further increased the core promoter activity to about 4-fold.

5′ deletions of the regulatory element (see FIGS. 5(A) and (B) and analysis by transient expression using biolistics showed that the regulatory element was active within a fragment 62 bp from the transcriptional start site (position 1992 of SEQ ID NO: 1 or 22) indicating that the core promoter has a basal level of expression (see Table 5: FIGS. 5(D) and (I)).

TABLE 5
Transient GUS activity detected in soybean leaves by staining
with X-gluc after particle bombardment. Vectors illustrated
in FIGS. 5 (A) and (B).

| | (nucleotides) | |
|---|---|---|
| Genes | SEQ ID NO: 1 or 22 | GUS staining |
| 1. T1275-GUS-nos | (1-2224) | + |
| 2. −1639-GUS-nos | (705-2224) | + |
| 3. −1304-GUS-nos | (1040-2224) | + |
| 4. −684-GUS-nos | (1370-2224) | + |
| 5. −394-GUS-nos | (1660-2224) | + |
| 6. −197-GUS-nos | (1875-2224) | + |
| 7. −62-GUS-nos | (1992-2224) | + |
| 8. −62(−tsr)-GUS-nos | | + |
| 9. −12-GUS-nos | (2042-2224) | + |
| 10. +30-GUS-nos | — | − |

Deletion of a fragment containing the transcriptional start site (see—62(-tsr)/GUS/nos in FIG. 6(B), Table 5) did not eliminate expression, however deletions to +30 (+30-GUS_nos) reduced expression dramatically. Similar results were obsereved in transgenic tomato (see below; FIGS. 5(H) and (I)) indicating that the region defined from about −12 to about +30 contained the core promoter. DNA sequence searches did not reveal conventional core promoter motifs within this region as are typically found in plant genes, such as the TATA box.

Deletion of a fragment containing the transcriptional start site (see—62(-tsr)-GUS-nos in FIGS. 5(B), (H) and (I); Table 5, Examples) reduced expression dramatically in transgenic tomato, however deletions to +30 did eliminate expression indicating that the region defined from about −12 to about +30 bp contained the core promoter. Deletion of sequences surrounding the transcriptional start site, reduced activity to about 2% of the activity associated with the −62-GUS construct, indicating that the transcriptional start site sequence is required for tCUP regulatory element activity.

A number of the 5′ regulatory element deletion clones (FIG. 5(A)) were transferred into tobacco by Agrobacterium-mediated transformation using the vector pRD400. Analysis of GUS specific activity in leaves of transgenic plants (see Table 6) confirmed the transient expression data down to the −197 fragment (nucleotide 1857 of SEQ ID NO: 1).

TABLE 6
GUS specific activities in leaves of greenhouse-grown transgenic
tobacco, SR1, transformed with the T1275-GUS-nos gene fusion
and 5′ deletion clones (see FIG. 5 C). Mean ± SE(n)

| | nucleotides | GUS specific activities |
|---|---|---|
| Genes | SEQ ID NO: 1 or 22 | pmoles MU/min/mg protein |
| 1. T1275-GUS-nos | (1-2224) | 283 ± 171 (27) |
| 2. −1639-GUS-nos | (705-2224) | 587 ± 188 (26) |
| 3. −1304-GUS-nos | (1040-2224) | 632 ± 217 (10) |
| 4. −684-GUS-nos | (1370-2224) | not determined |
| 5. −394-GUS-nos | (1660-2224) | 1627 ± 340 (13) |
| 6. −197-GUS-nos | (1875-2224) | 475 ± 74 (27) |

Histochemical analysis of organs sampled from the transgenic plants indicated GUS expression in leaf, seeds and flowers.

Deletions in the upstream region indicate that negative regulatory elements and enhancer sequences exist within the full length regulatory region. Deletion of the 5′ region to BstYI (−394 relative to the transcriptional start site) resulted in a 3 to 8 fold increase in expression of the gene associated therewith (Table 6), indicating the occurrence of at least one negative regulatory element within the XbaI-BstYI portion of the full length regulatory element. Other negative regulatory elements also exist within the XbaI-BstYI fragment as removal of an XbaI-PstI fragment also resulted in increased activity (−1304-GUS-nos; Table 6).

To determine if enhancer elements exist, fragments −394 to −62 (nucleotides 1660 to 1992 of SEQ ID NO:1) and −197 to −62 (nucleotides 1875 to 1992 of SEQ ID NO: 1) were fused to the −46 35S core promoter. Both fragments raised the expression of the core promoter about 150 fold (FIG. 5(D), constructs DRA1-35S and BST1-35S). Doubling of the −394 to −62 region (nucleotides 1660 to 1992 of SEQ ID NO:1) resulted in a 1.8 fold increase in GUS activity when fused to T1275 core promoter (BST1-GUS (−394-GUS) v. BST2-GUS; FIG. 5(D)), a similar effect is observed when the −394 to −62 region is double and fused to the 35S core promoter (BST1-35S v. BST2-35S). Doubling of the −197 to −62 fragment (nucleotides 1875 to 1992 of SEQ ID NO: 1) also produced increased GUS activity when fused to the T1275 core promoter (DRA2-GUS).

The −197 to −62 fragment (nucleotides 1875 to 1992 of SEQ ID NO:1; DRA 1-35S), the −197 to −62 fragment in reverse orientation, or inverted (DRA1R-35S), and a repeat of the −197 to −62 fragment (DRA2-35S) were also fused with the 35S minimal promoter (FIG. 5(E) and used to transform Arabidopsis.

Arabidopsis plants with immature floral buds and few silques were transformed with the above constructs by dipping the plant into a solution containing Agrobacterium tumefaciens, 2.3 g/L MS, 5% (w/v) sucrose and 0.03% Silwet L-77 (Lehle Seeds, Round Rock, Tex.) for 1-2 min, and allowing the plants to grow and set seed. Seeds from mature plants were collected, dried at 25° C., and sown on sterile media containing 40 μg/mL kanamycin to select transformants. Surviving plantlets were transferred to soil, grown and seed collected.

Constructs comprising the −197 to −62 fragment (nucleotides 1875 to 1992 of SEQ ID NO: 1) in regular or inverted orientation exhibited increased transcriptional enhancer activity, over that of the minimal promoter (FIG. 5(F). A further increase in activity was observed when plants were transformed with constructs comprising repeated regions of this regulatory element (FIG. 5(F). Tissue staining of transformed plants expressing DRA 1-35S indicated that this construct was expressed constitutively as it was detected in all tested organs, including flower, silque and seedling (FIG. 5(G)).

RENT (Repetitive Element from N. tabacum) Family of Repetitive Elements

An amplified N. tabacum line SRI custom library (Stratagene), which contained MboI partially digested genomic DNA in the 8-DashII vector, was screened by hybridization with ³²P-labelled probe fragment 5 (probe 5 is a BstYI-SmaI fragment of T1275, nucleotides 1660-2224 of SEQ ID NO:1, see FIG. 5(C)) at 65° C. over night (16-20 hours in Churches buffer: 7% SDS; 0.5M NaPO₄; 10 mM EDTA) and washing at 50° C. in 0.1×SSC, 0.1% SDS for 60 minutes, or two washes of 20-30 minutes each. Approximately 70 clones were identified in this manner. The restriction fragment of each 8 insert which hybridized with probe fragment 5 on a Southern blot, hybridized at 65° C. (overnight; 16-20 hours) and washed at 60° C., 0.1×SSC, 0.1% SDS (for 60 minutes, or two washes of 20-30 minutes each; stringent hybridization conditions), was gel purified with EluQuick (Schleicher and Schuell) and subcloned into pGEM4Z (Promega). Both strands of each subclone were sequenced with universal or custom designed primers, as appropriate. From this screen, 5 clones were obtained for further analysis. Approximately 2 to 3 kb of each genomic clone was subcloned and overlapping sequences obtained. These clones are called RENT 1, 2, 3, 5 and 7.

Two primers, approximately 30 basepairs in length were synthesized (Synthaid Biotechnologies Inc.), one in the forward direction at position 1707 of the T1275 nucleotide sequence and the other in the reverse direction at position 2092. Each incorporated a convenient restriction site, the first a HindIII site:

| HindIII primer: | TTA AGA TTT AAT Taa gct tAT AAT TAC AAA | (SEQ ID NO:19) |
|---|---|---|
| and the second a BglII site: | | |
| BglII primer: | ATT Cag atc tGG CGG TTGGTG AGA AA. | (SEQ ID NO:20) |

The primers were then used for PCR amplification of each of the five RENT fragments with attached restriction sites using Taq DNA polymerase (from MBI Fermentas Inc). The protocol accompanying the modifying enzyme was followed, with a reduction to 0.2 ul in the amount of Taq DNA polymerase used, in a total reaction mix of 50 μl. The fragment from the original T1275 sequence was also amplified.

All PCR products were electrophoresed on a 1% TAE agarose gel and visualized by staining with ethidium bromide. The 400 basepair band representing the PCR product was excised and purified. Each DNA sample was then digested with Hind III/Bgl II and concentrated in an overnight precipitation with one half volume of 7.5M ammonium acetate and 2 volumes of 95% ethanol.

A plasmid containing the vector, pTZ19R, containing the tCUP delta regulatory element, with a Kozak sequence was also digested with Hind III/Bgl II, electrophoresed on a 1% agarose gel and gel purified. Briefly, tCUP delta (see below, description relating to Table 10 and FIG. 10) was created by replacing the NdeI site (FIG. 10(A)) within the leader sequence to a BglII site thereby eliminating the upstream ATG at position 2087 of SEQ ID NO: 1. A Kozak consensus sequence was also constructed at the initiator MET codon and a NcoI site was added to facilitate construction with other coding regions (see FIG. 10(B)). Nucleotides 1-86 of SEQ ID NO:3 (i.e. tCUP delta with Kozack sequence) are derived from T1275 (nucleotides 2086-2170 of SEQ ID NO:1), and a Kozack sequence from nucleotides 87 to 97 of SEQ ID NO:3. Nucleotides 98 to 126 of SEQ ID NO:3 comprise the vector sequence between the enhancer fragment and the GUS ATG. The GUS ATG is located at nucleotides 127-129 of SEQ ID NO:3.

Each of the five RENT PCR fragments, as well as the T1275 control PCR fragment was ligated into the digested plasmid, in a 4 to 1, insert to vector ratio. These were transformed into Top10 competent cells (Invitrogen Corp.) via electroporation using an Invitrogen electroporator and their supplied protocol. The transformed cells were plated on ampicillin containing LB plates and allowed to grow overnight. The colonies were then grown overnight in liquid LB plus ampicillin to be used for plasmid isolation using the Wizard Plasmid Miniprep Kit (Promega Corp.) or the Qiaprep Spin Miniprep Kit (Qiagen Inc.). Isolated plasmids were restricted with Hind III, Bgl II and Hind III/Xba I to verify restriction patterns. Once these were ascertained to be correct, the insert containing plasmids were sequenced. Therefore, the regulatory elements used for the analysis in FIG. 14(A), including tCUP-RENT, consist of the amplified PCR fragment fused to tCUP delta comprising a Kozak sequence. The 35S-46 construct used for the analysis presented in FIG. 14(A) was prepared by generating a −46 minimal 35S promoter (−46-35S) was generated by PCR using the primer pair:

| | | |
|---|---|---|
| 46-35S-1 primer: | CACTCTGCAGGCAAGACCCTTCCTCTATA, | (SEQ ID NO:13) |
| and | | |
| | ATATAAGCTTTGGGGTTTCTACAGGACG, | (SEQ ID NO:14) |

and pBI221 (Clontech) DNA as a template. The PCR product was digested with PstI and BamHI, and the resulting fragment was used to replace the PstII and BamHI fragment in pBI221.

Approximately 2 to 3 kb region of each genomic clone, which on Southern blots hybridized with probe 5 (a BstYI-SmaI fragment) was subcloned and overlapping sequence reads were obtained on both DNA strands of each subclone. Sequence analysis indicated the presence of sequence similarity, but not identity, along the 3′ ends of these subclones, with divergence at the 5′ ends. The 5′ ends of the clones all diverged at the same position. These data suggest that each independent clone represented a different member of the RENT repetitive element family interrupting different regions of the genome. Moreover, all five subclones studied were similar to the tCUP sequence in the region which delimits maximal regulatory element activity and is situated towards one end of RENT. The five subclones exhibited 77 to 92% (FIGS. 13(A)-(C)) with sequence similarity with the tCUP sequence in the probe 5 region (1724-2224 of SEQ ID NO: 1) which confers regulatory element activity. The repetitive elements also do not appear to be present in close tandem locations as probe five hybridized only once with each genomic clone.

Therefore, t-CUP is a member of a large family of repetitive elements in Nicotiana tabacum (RENT) in which the regions essential for regulatory element activity have been conserved. All RENT sequences, including tCUP share a common sequence of ca. 525 bp from transcriptional start site of t-CUP (1724-2224 of SEQ ID NO: 1). RENT sequences 1, 2, 3, 5 and 7 had high homology among themselves, outside of this 525 bp region (FIGS. 13(A) and (B)).

The following fragments of the members of the RENT family, including the SEQ ID NO: 1, have been characterized, and their utility demonstrated herein. For example, the fragment comprising nucleotides:
1660-1992 (−394 to −62 fragment) enhances expression of the -46 minimal promoter of 35S, and a fragment of T1275 (see Bst1-GUS; Bst1-35S, Bst2-GUS, Bst2-35S, of FIG. 5D);
1660-1875 (BstYI-DraI fragment; see FIG. 5C; −394 GUS-nos; and Table 6) The data in Table 6 indicates that this fragment acts as an enhancer;
1660-2224 (BstYI-SmaI fragment; see FIG. 5C; −394-GUS-nos) The activity of this fragment is described in Tables 5 and 6;
1724-2224 (FIG. 13C, and FIG. 14A, tCUP RENT);
1875-2086 (DraI-NdeI fragment; core promoter element), see FIG. 5C and Table 6 (−197-GUS-nos);
1875-1992 (DraI-62 fragment) This fragment is shown to enhance expression of the −46 minimal promoter of 35S, and a fragment of T1275, as shown in FIG. 5D (see DraI-GUS; Dra2-GUS; Dra1-35S; Dra2-35S), and FIGS. 5E-G (Dra1-35S; Dra2-35S), and functions as a transcriptional enhancer;
2084-2224 (NdeI-SmaI fragment, or “N”; Tables 10-12, FIG. 5B (+30-GUS-nos), FIG. 7 (T1275-GUS-nos; 35S-GUS-nos), and FIG. 11 (35S+N-GUS-nos);
2091-2170 (AN) see Tables 10-12.

Based on sequence similarity using NCBI Blast 2 analysis (default parameters: blastn matrix, Lambda=1.37, K=0.71 1, H=1.31), the fragments identified in above, exhibit from about 90 to 98% identity to similar length fragments of the RENT sequences (SEQ ID NO's: 5-9).

To verify the number of repetitive elements in the region giving rise to regulatory element activity, more precise measurements were performed using slot blot hybridization. Slot blots were probed under conditions of high stringency level as used for the Southern blot (data not presented). These results indicate that a range of approximately 10 to 43 copies of similar repetitive elements were estimated per haploid genome of N. tabacum. When the same slot blots were washed at lower stringency, the same stringency as used during library screening, a range of approximately 62 to 199 copies of similar repetitive elements were estimated per haploid genome.

RNase protection assays and probes spanning both strands of the combined tCUP and downstream sequence region, in the areas encompassing probes 5 to 8 (probe 5 was a 578 bp BstYI-SmaI fragment; probe 6 was a 574 bp RsaI-RsaI fragment; probe 7 was a 244 bp RsaI-RsaI fragment; and probe 8 was a 321 bp Rsa1-XbaI fragment) did not result in any protection in the repetitive region. RNase protection assays performed under these conditions has previously been shown to tolerate single mismatches by protection of non-identical sequences. This suggests that protected fragments may be detectable if members of the RENT family were transcribed, at least for those elements that exhibit high sequence similarity. Examples of those elements which may be detectable are those hybridizing at high stringency on blots or those from which the downstream PCR clones originated. A lack of open reading frames was observed within the RENT sequences. Together, this suggests a lack of coding capacity within the sequenced region.

Thus the tCUP cryptic, constitutive regulatory element is contained within a moderately repeated repetitive element, which is the first known member of a new repetitive element family.

Protoplast Isolation, Electroporation and Culture

Plasmids, prepared as described above were amplified and isolated to produce a sufficient amount of DNA necessary for transient expression in pea protoplasts, using the Qiagen Plasmid MidiKit (Qiagen Inc.).

Pea (Pisum sativum L. var. Laxton Progress) seedlings were grown in soil at 18° C. (16 hr light, 8 hr dark; 15-20 μmol m⁻² s⁻¹) provided by Philips (USA) F20 T12 ‘cool white’ flourescent tubes and young fully expanded leaves were harvested from 2-3 weeks old plants. Leaves surface sterilized 5 minutes in 5% commercial bleach (Javex) (1% NaOCl). The abaxial surface of leaves were gently rubbed with carborundum powder, rinsed three times with sterile water, midribs removed and remaining leaf blade was cut by sharp razor into ca 1 cubic cm pieces and floated rubbed surface facing first enzyme solution containing 0. 1% (w/v) pectolyase Y-23 (Seishin Pharmaceutical, Japan), 0.5% potassium dextran sulphate (Calbiochem, USA) and 0.5 M mannitol (pH 5.5) and vacuum infiltrated for 15 minutes. The leaf tissues were then incubated at 26 ° C. for another 15 minutes on a shaker at 60 excursions/min. The solution was then decanted by filtration through a 100 mesh nylon filter and the remaining tissue was incubated for 1-1.5 hr in a second enzyme solution containing 1.0% (w/v) Cellulase Onozuka R-10 (Yakult Honsha, Japan), Pectolyase Y-23 0.05% (w/v) (Seishin Pharmaceutical, Japan and 0.5 M mannitol, pH 6.0 at 26 C with 60 excursions/min.

The protoplasts were collected by filtration through a 100 μm nylon mesh filter followed by centrifugation at 500 rpm for 5 min. The protoplasts were gently collected by a wide bore pippet and washed twice with W5 electroporation buffer (4.5 g NaCl, 0.5 g glucose, 9.2 g CaCl₂, 2.0 g KC in 500 ml) for 5 min at 500 rpm and finally suspended at approximately 1×16⁶/ml in 0.5 M mannitol containing 150 μM MgCl₂.

The viability of protoplasts was confirmed by FDA (Fluorescein diactate) and alanine blue staining and protoplasts were kept on ice for 30 minutes prior to electroporation. A 25-30 μg luciferin and desired DNA was added to 500 μl protoplast suspension, mixed gently and electroporated at 100 μF and 200 v using Gene Pulser II (BioRad). The electroporated protoplasts were kept on ice for 15-30 min, centrifuged for 5 min at 500 rpm and mixed with 0.5 ml growth medium. The cultures were kept in dark at 25° C. for 24 hr.

To each 500 μl of protoplast suspension 200 μl of buffer solution containing 100 mM KPO₄, 1 mM EDTA, 10% glycerol, 0.5% triton x-100, 7 mM β-merceptoethanol was added and protoplasts were lysed and luciferase and GUS activities were measured as described in Jefferson 1987 and Mathews et al., 1995 (Jefferson, R. A. 1987. Assaying chimeric genes in plants: the GUS fusion system. Plant Mol. Biol. Reporter 5:387-405; Mathews, F. B., Saunders J. A., Gebhardt J. S., Lin J-J., and Koehler M. 1995. Reporter genes and transient assays for plants. In “Methods in Molecular Biology, Vol 55: Plant Cell Electroporation and Electrofusion Protocols” ed. J. A. Nickoloff Humana Press Inc., Totowa, N.J. pp.147-162). All GUS activities were normalized with respect to luciferase activities to account for variation caused by electroporation.

When RENT sequences were cloned and tested for GUS transient gene expression, all RENT sequences demonstrated high regulatory element activity (FIG. 14(A)).

FIG. 14(A) shows that each of the regulatory elements isolated from the 5 RENT sequences (RENT 1, 2, 3, 5, 7 and tCUP-RENT) is capable of driving the expression of a coding region of interest (in this case GUS) with which they are in operative association. The RENT regulatory elements resulted in more GUS activity than that observed with the 35S minimal promoter-GUS construct (35S-46; FIG. 14 (A)). Furthermore, these results demonstrate that the RENT regulatory sequences are active in a heterologous species (pea).

Constitutive Gene Expression by −394t-CUP Sequence in Transgenic Arabidopsis thaliana L.

Arabidopsis thaliana (ecotype Columbia) was grown in a growth chamber (16 hr of light and 8 hr of darkness at 23° C.). Plants with immature floral buds and few siliques were dipped into a solution containing Agrobacterium tumefaciens, 2.3 g/L MS salts (Sigma), 5% (w/v) sucrose and 0.03% Silwet L-77 (Lehle Seeds, Round Rock, Tex.) for 0.5 min. T1 seeds were collected, dried at 25° C., and sown on sterile media containing 40 μg/mL kanamycin to select the transformants. Surviving T1 plantlets were transferred to soil and used for the analysis of GUS activity. For histochemical GUS assay, tissue was incubated in a 0.5 mg/ml solution of 5-bromo-4-chloro-indolyl β-D-glucuronide in 100 mM sodium phosphate buffer, pH 7.0, infiltrated in a vacuum for half a hour and incubated at 37° C. overnight. Following the incubation, tissue was washed in 70% ethanol to clear off chlorophyll.

Arabidopsis plants were transformed with −394t-CUP-GUS fusion gene. This fragment of tCUP exhibits substantial homology with the other identified RENT sequences (FIG. 13(B). The result, presented in FIG. 14(B), demonstrates that the −394t-CUP sequence drive constitutively GUS reporter gene expression in all organs such as leaves, stem, roots, and floral organs in transgenic Arabidopsis. Since this region is common to the characterized RENT sequences these results indicate that all RENT sequences contain regulatory elements capable of regulating constitutive gene expression.

Activity of the T1275 Regulatory Element

Analysis of leaves of randomly-selected, greenhouse-grown plants regenerated from culture revealed a wide range of GUS specific activities (FIG. 6(A); T plants). Plants transformed with pBI 121 (CLONETECH) which contains the 35S-GUS-nos gene yielded comparable specific activity levels (FIG. 6(A); S plants). Furthermore, the GUS protein levels detected by Western blotting were similar between plants transformed with either gene when the GUS specific activities were similar (FIG. 6(C)).

Generally, the level of GUS mRNA in the leaves as determined by RNase protection (FIG. 6(B)) correlated with the GUS specific activities, however, the level of GUS mRNA was about 60 fold (mean of 13 measurements) lower in plants transformed with the T1275-GUS-nos gene (FIG. 6(B)) when compared with plants transformed with 35S-GUS-nos.

Since the levels of protein and the activity of extractable protein were similar in plants transformed with T1275-GUS-nos or 35S-GUS-nos, yet the mRNA levels were dramatically different, these results suggested the existence of a regulatory element downstream of the transcriptional start site in the sequence of T1275-derived transcript.

Post-Transcriptional Regulatory Elements within T1275

An experiment was performed to determine the presence of a post-transcriptional regulatory element within the T1275 leader sequence. A portion of the sequence downstream from the transcriptional initiation site was deleted in order to examine whether this region may have an effect on translational efficiency (determined by GUS extractable activity), mRNA stability or transcription.

Deletion of the Nde1-Sma1 fragment (“N”; SEQ ID NO:2) from the T1275-GUS-nos gene (FIG. 15; T1275-N-GUS-nos; includes nucleotides 2084-2224 of SEQ ID NO: 1) resulted in at least about 46-fold reduction in the amount of GUS specific activity that could be detected in leaves of transgenic tobacco cv Delgold (see Table 7). Similar results, of about at least a 40 fold reduction in GUS activity due to the deletion of the Nde1-Sma1 fragment, were observed in transgenic tobacco cv SR1 and transgenic alfalfa (Table 7). Addition of the same fragment (Nde1-Sma1) to a 35S-GUS-nos gene (FIG. 7; 35S+N-GUS-nos) construct increased the amount of GUS specific activity by about 5-fold in tobacco, and by a much higher amount in alfalfa (see Table 7).

TABLE 7
GUS specific activity in leaves of greenhouse-grown transgenic
tobacco cv Delgold, SR1 and transgenic alfalfa transformed
with vectors designed to assess the presence of cryptic
regulatory sequences within the transcribed sequence derived
from the T1275 GUS gene fusion (see FIG. 7). Mean ± SE(n).

| | GUS specific activity | | | |
|---|---|---|---|---|
| | pmoles MU/min/mg protein | | | |
| Construct | Delgold (1) | Delgold (2) | SR1 | Alfalfa |
| T1275-GUS- | 557 ± 183 | 493 ± 157 | 805 ± 253 | 187 ± 64 |
| nos | (21) | (25) | (22) | (24) |
| T1275−N- | 12 ± 3 | 12 ± 3 | 6 ± 2 | 4 ± 0.5 |
| GUS-nos | (22) | (27) | (25) | (25) |
| 35S-GUS-nos | 1848 ± 692 | 1347 ± 415 | 1383 ± 263 | 17 ± 11 |
| | (15) | (26) | (25) | (24) |
| 35S+N-GUS- | 6990 ± 3148 | 6624 ± 2791 | 6192 ± 1923 | 1428 ± 601 |
| nos | (23) | (26) | (24) | (24) |

A similar effect was noted in organs tested from transformed tobacco (Table 8) and alfalfa plants (Table 9)

TABLE 8
Expression of T1275-GUS-nos (+N) compared with
T1275-(−N)-GUS-nos (−N) in organs of transgenic
tobacco cv. Delgol and SR1. Mean ± SE(n = 5).

GUS specific Activity (pmol MU/min/mg/protein)

| | Delgold | | SR1 | |
|---|---|---|---|---|
| Organ | +N | −N | +N | −N |
| Leaf | 1513 ± 222 | 35 ± 4 | 904 ± 138 | 4 ± 1 |
| Flower | 360 ± 47 | 38 ± 8 | 175 ± 44 | 28 ± 3 |
| Seed | 402 ± 65 | 69 ± 7 | 370 ± 87 | 33 ± 5 |

TABLE 9
Expression of T1275-GUS-nos, T1275-(−N)-GUS-nos, 35S-GUS-nos,
35S-GUS(+N)-GUS-nos in organs of transgenic alfalfa. Mean ± SE(n = 5).

| | GUS Specific Activity (pmol Mu/min/mg protein) | | | |
|---|---|---|---|---|
| Construct | Leaf | Petiole | Stem | Flower |
| T1275-GUS | 756 ± 73.6 | 1126 ± 72.7 | 1366.7 ± 260 | 456.1 ± 160.9 |
| T1275(−N)GUS | 5.4 ± 1.4 | 7.6 ± 1.2 | 8.1 ± 2.0 | 7.25 ± 1.7 |
| 35S-GUS | 67.5 ± 50.3 | 48.9 ± 23.2 | 56.8 ± 28.7 | 23.2 ± 7.3 |
| 35S(+N)GUS | 5545 ± 2015 | 10791 ± 6194 | 9931 ± 5496 | 1039 ± 476.7 |
| Control | 3.7 | 13.2 | 11.8 | 18.7 |

In transient expression assays using particle bombardment of tobacco leaves, the Nde1-Sma1 fragment fused to the minimal −46 35S promoter enhanced basal level of 35S promoter activity by about 80 fold (28.67±2.91 v. 0.33±0.33 relative units; No.blue units/leaf).

SEQ ID NO:2 comprises nucleotides 2084 to 2224 of SEQ ID NO: 1. Nucleotides 1-141 of SEQ ID NO:2 comprise nucleotides obtained from the plant portion of T1275 (nucleotides 2084 to 2224 of SEQ ID NO: 1). Nucleotides 142-183 of SEQ ID NO:2 comprise vector sequence between the enhancer fragment and the GUS ATG. The GUS ATG is located at nucleotides 186-188 of SEQ ID NO:2.

A shortened fragment of the NdeI-SmaI fragment (see SEQ ID NO:3), referred to as “ΔN”, “dN”, “deltaN” or “tCUP delta” and lacking the out-of frame upstream ATG at nucleotide 2087-2089 of SEQ ID NO: 1, was also constructed and tested in a variety of species. ΔN was created by replacing the NdeI site (FIG. 10(A)) within the leader sequence to a BglII site thereby eliminating the upstream ATG at position 2087 of SEQ ID NO: 1. A Kozak consensus sequence was also constructed at the initiator MET codon and a NcoI site was added to facilitate construction with other coding regions (see FIG. 10(B)). Nucleotides 1-86 of SEQ ID NO:3 (i.e. ΔN with Kozack sequence) are derived from T1275 (nucleotides 2086-2170 of SEQ ID NO:1). ΔN also includes a Kozack sequence from nucleotides 87 to 97 of SEQ ID NO:3, and nucleotides 98 to 126 of SEQ ID NO:3 comprise the vector sequence between the enhancer fragment and the GUS ATG. The GUS ATG is located at nucleotides 127-129 of SEQ ID NO:3.

Constructs comprising ΔN, for example T1275(ΔN)-GUS-nos, when introduced into tobacco yielded 5 fold greater levels of GUS activity in leaves of transgenic tobacco (5291±986 pmolMU/min/mg protein; (n=29) compared to plants expressing T1275-GUS-nos (1115±299 pmol MU/min/mg protein; n=29).

Activity of NdeI-SmaI, N, and ΔN in other Species

In monocots, transient expression in corn callus indicated that the NdeI-SmaI fragment (SEQ ID NO:2), or a shortened NdeI-SmaI fragment, ΔN (SEQ ID NO:3), significantly increases GUS expression driven by the 35 S promoter, but not to the higher level of expression generated in the presence of the ADH1 intron (“i”; FIG. 11 and Table 10).

TABLE 10
Transient expression analysis of GUS activity in bombarded corn
calli. Luciferase activity was used to normalize the data.
Mean ± se (n = 5).

| Construct | Ratio GUS:Luciferase activity |
|---|---|
| 35S GUS-nos | 7.4 ± 4 |
| 35S(+N)-GUS-nos | 19 ± 5 |
| 35S(ΔN)-GUS-nos | 18 ± 10 |
| 35S-i-GUS-nos | 66 ± 27 |

The functionality of the NdeI-SmaI fragment (SEQ ID NO:2) was also determined in non-plant species. In conifers, for example white spruce, transient bombardment of cell culture exhibited an increase in expression (Table 11).

TABLE 11
Expression of T1275-GUS-nos, T1275(−N)-GUS-nos,
35S-GUS-nos, 35S (+N)-GUS-nos in white spruce embryonal
masses following bombardment (n = 3).

| | Average GUS expression per leaf |
|---|---|
| Construct | (Number of blue spots) |
| T1275-GUS-nos | 72.67 ± 9.33 |
| T1275(−N)-GUS-nos | 21.33 ± 4.49 |
| 35S-GUS-nos | 113.67 ± 17.32 |
| 35S(+N)-GUS-nos | 126.33 ± 19.41* |

*average spot much greater in size and strength.

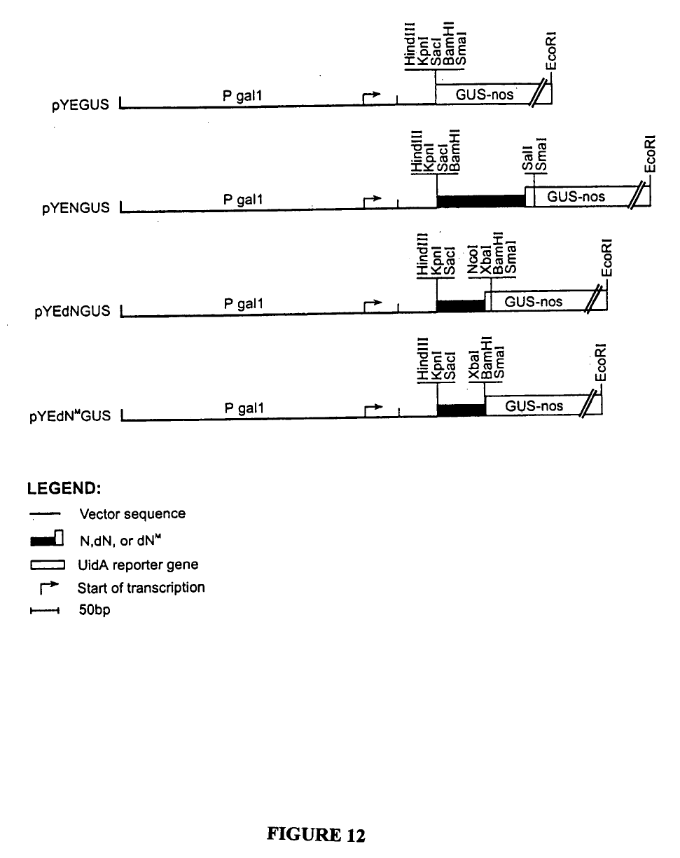

In yeast, the presence of the NdeI-SmaI fragment (SEQ ID NO:2) or ΔN (SEQ DI NO:3) exhibited strong increase in expression of the marker gene. A series of constructs comprising a galactose inducible promoter P_{gal1}, various forms of the Nde1-Sma1 fragment, and GUS (UidA) were made within the yeast plasmid pYES2. A full length Nde1-Sma1 fragment N (pYENGUS), ΔN (containing a Kozak consensus sequence; pYEdNGUS), and ΔN without a Kozak consensus sequence (pYEdN^{M}GUS; or ΔN^{M}) were prepared (see FIG. 12, and SEQ ID NO:4).

Nucleotides 1-86 of SEQ ID NO:4 (ΔN^{M}) comprise a portion of the enhancer regulatory region obtained from T1275 (nucleotide 2086-2170 of SEQ ID NO:1), while nucleotides 87-116 comprise a vector sequence between the enhancer fragment and the GUS ATG which is located at nucleotides 117-119 of SEQ ID NO:4.

These constructs were tested in yeast strain INVSC1 using known transformation protocols (Agatep R. et al. 1998; biomednet.com/db/tto). The yeast were grown in non-inducible medium comprising raffinose as a carbon source for 48 hr at 30° C. and then transferred onto inducible medium (galactose as a carbon source). Yeast cells were harvested after 4 hr post induction and GUS activity determined quantitatively. Up to about a 12 fold increase in activity was observed with constructs comprising ΔN. Constructs comprising ΔN^{M} exhibited even higher levels of reporter activity. The results indicate that the Nde1-Sma1 fragment (SEQ ID NO:2), ΔN (SEQ ID NO:3) and ΔN^{M} (SEQ ID NO:4) are functional in yeast (Table 12).

TABLE 12
Expression of pYEGUS, pYENGUS, pYEdNGUS, and
pYEdN^{M}GUS (ΔN, without a Kozak consensus sequence)
in transformed yeast (n = 5).

| | Expt. 1 | Expt. 2 |
|---|---|---|
| Construct | Activity | Activity |
| pYES-GUS-nos | 93 ± 15 | 407 ± 8 |
| pYES(+N)-GUS-nos | 753 ± 86 | 1771 ± 191 |
| pYES(ΔN)-GUS-nos | 1119 ± 85 | 2129 ± 166 |
| pYES(ΔN^{M})-GUS-nos | 1731 ± 45 | 6897 ± 536 |

Constructs containing ΔN^{M} (i.e. ΔN lacking the Kozack sequence; SEQ ID NO:4) were also tested in insect cells. These constructs comprised the insect virus promoter ie2 (Theilmann D. A and Stewart S., 1992, Virology 187: pp. 84-96) in the present or absence of ΔN^{M} and CAT (chloramphenicol acetyl-transferase) as the reporter gene. The insect line, Ld652Y, derived from gypsy moth (Lymantria dispar) was transiently transformed with the above constructs using liposomes (Campbell M. J. 1995, Biotechniques 18: pp. 1027-1032; Forsythe I. J. et al 1998, Virology 252: pp. 65-81). Cells were harvested 48 hours after transformation and CAT activity quanitatively measured using tritiated acetyl-CoA (Leahy P. et al. 1995 Biotechniques 19: pp. 894-898). The presence of the translational enhancer was found to significantly modulate the activity of the insect promoter-reporter gene construct in insect cells.

Bacteria were transformed with either pBI221, comprising 35S promoter and GUS, or 35S-N-GUS, comprising the full length Nde1-Sma1 fragment (SEQ ID NO:3). Since uidA (GUS) is native to E.coli, two uidA mutants, uid1 and uidA2, that do not express uidA, were used for these experiments (mutants obtained from E.coli Genetic Center 335 Osborn Memorial Laboratories, Department of Biology, Box 208104, Yale University, New Haven Conn. 06520-8104). These bacteria were transformed using standard protocols, and transformants were assessed by assaying GUS activity from a 50 μl aliquot of an overnight culture. The “N” fragment (35s-N-GUS) was observed to modulate the activity of the reporter gene in bacterial cells.

These data are consistent with the presence of a post-transcriptional regulatory sequence in the NdeI-SmaI fragment.

The NdeI-SmaI Fragment Functions as a Transcriptional Enhancer or mRNA Stability Determinant

The levels of mRNA were determined in leaves obtained from plants transformed with either T1275-GUS-nos, T1275-N-GUS-nos, 35S-GUS-nos, or 35S+N-GUS-nos (FIG. 9(A)). Relative RNA levels were determined by ribonuclease protection assay (Ambion RPAII Kit) in the presence of α-³²P-CTP labeled in vitro transcribed probe and autoradiographic quantification using Kodak Digital Science 1D Image Analysis Software. Hybridization conditions used during RNase protection assay were overnight at 42-45 degrees in 80% formamide, 100 mM sodium citrate pH 6.4, 300 mM sodium acetate pH 6.4, 1 mM EDTA.

The levels of mRNA examined from transgenic tobacco plants transformed with either T1275-GUS-nos, T1275-N-GUS-nos, 35S-GUS-nos, or 35S+N-GUS-nos, were higher in transgenic plants comprising the NdeI-SmaI fragment under the control of the T1275 regulatory element but lower in those under the control of the 35S promoter, than in plants comprising constructs that lack this region (FIG. 7(A)). This indicates that this region functions by either modulating transcriptional rates, or the stability of the transcript, or both.

The NdeI-SmaI Fragment Functions as a Translational Enhancer

Analysis were performed in order to determine whether the NdeI-SmaI region functions post-transcriptionally. The GUS specific activity:relative RNA level was determined from the GUS specific activity measurements, and relative RNA levels in greenhouse grown transgenic plants (FIG. 9(B)). The ratio of GUS specific activity to relative RNA level in individual transgenic tobacco plants comprising the NdeI-SmaI fragment is higher than in plants that do not comprise this region (FIG. 9(B)). Similar results are obtained when the data are averaged, indicating an eight fold reduction in GUS activity per RNA. Similarly, an increase, by an average of six fold, in GUS specific activity is observed when the NdeI-SmaI region is added within the 35S untranslated region (FIG. 9(B)). The GUS specific activity:relative RNA levels are similar in constructs containing the NdeI-SmaI fragment (T1275-GUS-nos and 35S+N-GUS-nos). These results indicate that the NdeI-SmaI fragment (SEQ ID NO:2) modulates gene expression post-transcriptionally.

Further experiments, involving in vitro translation, suggest that this region is a novel translational enhancer. For these experiments, fragments, from approximately 3′ of the transcriptional start site to the end of the terminator, were excised from the constructs depicted in FIG. 7 using appropriate restriction endonucleases and ligated to pGEM4Z at an approximately similar distance from the transcriptional start site used by the prokaryotic T7 RNA polymerase. Another construct containing the AMV enhancer in the 5′ UTR of a GUS-nos fusion was similarly prepared. This AMV-GUS-nos construct was created by restriction endonuclease digestion of an AMV-GUS-nos fusion, with BglII and EcoRI, from pBI525 (Datla et al., 1993, Plant Science 94: 139-149) and ligation with pGEM4Z (Promega) digested with BamHI and EcoRI. Transcripts were prepared in vitro in the presence of m⁷G(5′)ppp(5′)G Cap Analog (Ambion). Transcripts were translated in vitro in Wheat Germ Extract (Promega) in the presence of 35S-Methionine and fold enhancement calculated from TCA precipitable cpms.

Translation of transcripts in vitro demonstrate an increase in translational efficiency of RNA containing the NdeI to SmaI fragment (see Table 13).

TABLE 13
In vitro translation of mRNA obtained from transgenic tobacco
plants transformed with vectors with or without a NdeI-SmaI
fragment obtained from the T1275 GUS gene fusion
(see FIG. 7) using wheat germ extract.

| | in vitro translation |
|---|---|
| in vitro transcript | fold enhancement |
| T1275-GUS-nos | 3.7 |
| T1275-N-GUS-nos | 1 |
| AMV-GUS-nos | 1.9 |

The levels of protein produced using mRNAs comprising the NdeI-SmaI fragment are also greater than those produced using the known translational enhancer of Alfalfa Mosaic Virus RNA4 (Jobling S. A. and Gehrke L. 1987, Nature, vol 325 pp. 622-625; Datla R. S. S. et al 1993 Plant Sci. vol 94, pp. 139-149). These results indicate that this region functions post-transcriptionally, as a translational enhancer.

All citations are hereby incorporated by reference. The nucleic acid sequences listed in the Sequence Listing filed herewith are incorporated by reference into this application in their entireties.

The present invention has been described with regard to one or more embodiments. However, it will be apparent to persons skilled in the art that a number of variations and modifications can be made without departing from the scope of the invention as defined in the claims.

1. An isolated nucleotide sequence comprising the nucleic acid sequence defined by SEQ ID NO:22, a nucleotide sequence that hybridizes to the nucleic acid sequence of SEQ ID NO:22, or a nucleotide sequence that hybridizes to a compliment of the nucleotide sequence of SEQ ID NO:22, wherein hybridization condition is selected from the group consisting of
hybridizing overnight in a solution comprising 7% SDS, 0.5M NaPO4 buffer at pH 7.2, and 10 mM EDTA at 65° C. and washing for one hour at 60° C. in a solution comprising 0.1×SSC and 0.1% SDS;
hybridizing overnight in a solution comprising 7% SDS, 0.5M NaPO4 buffer at pH 7.2, and 10 mM EDTA at 65° C. and washing for one hour at 65° C. in a solution comprising 2×SSC and 0.1% SDS; and
hybridizing overnight in a solution comprising 4×SSC at 65° C. and washing one hour in 0.1×SSC at 65° C., and
wherein the nucleotide sequence exhibits regulatory element activity.

2. The isolated nucleotide sequence of claim 1, wherein the nucleotide sequence is defined by SEQ ID NO: 1, a nucleic acid sequence that hybridizes to the nucleotide sequence of SEQ ID NO: 1, or a nucleic acid sequence that hybridizes to a compliment of the nucleotide sequence of SEQ ID NO: 1.

3. The isolated nucleotide sequence of claim 1, wherein the nucleotide sequence is defined by SEQ ID NO:21, a nucleic acid sequence that hybridizes to the nucleotide sequence of SEQ ID NO :21, or a nucleic acid sequence that hybridizes to a compliment of the nucleotide sequence of SEQ ID NO:21.

4. The isolated nucleotide sequence of claim 1, wherein the nucleotide sequence is selected from the group consisting of SEQ ID NO:1, SEQ ID NO:5, SEQ ID NO:6, SEQ ID NO:7, SEQ ID NO:8, SEQ ID NO:9 and SEQ ID NO:21.

5. An isolated nucleotide sequence comprising the nucleic acid sequence defined by nucleotides 1660-1875 of SEQ ID NO: 1, a nucleotide sequence that hybridizes to nucleotides 1660-1875 of SEQ ID NO:1, or a nucleotide sequence that hybridizes to a compliment of nucleotides 1660-1875 of SEQ ID NO: 1, wherein hybridization condition is 65° C. over night in 7% SDS; 0.5M NaPO₄; 10 mM EDTA, followed by two washes at 50° C. in 0.1×SSC, 0.1% SDS for 30 minutes each, wherein the nucleotide sequence exhibits regulatory element activity.

6. The isolated nucleotide sequence of claim 5, wherein the nucleotide sequence is defined by nucleotides 1660-1992 of SEQ ID NO:1.

7. An isolated nucleotide sequence comprising the nucleic acid sequence defined by nucleotides 2091-2170 of SEQ ID NO:1, a nucleotide sequence that hybridizes to nucleotides 2091-2170 of SEQ ID NO: 1, or a nucleotide sequence that hybridizes to a compliment of nucleotides. 2091-2170 of SEQ ID NO: 1, wherein hybridization condition is 65° C. over night in 7% SDS; 0.5M NaPO₄; 10 mM EDTA, followed by two washes at 50° C. in 0.1×SSC, 0.1% SDS for 30 minutes each, wherein the nucleotide sequence exhibits regulatory element activity.

8. The isolated nucleotide sequence of claim 7, wherein the nucleotide sequence is defined by nucleotides 1660-2224 of SEQ ID NO: 1.

9. The isolated nucleotide sequence of claim 7, wherein the nucleotide sequence is defined by nucleotides 1723-2224 of SEQ ID NO: 1.

10. The isolated nucleotide sequence of claim 7, wherein the nucleotide sequence is defined by nucleotides 415-2224 of SEQ ID NO:1.

11. The isolated nucleotide sequence of claim 7, wherein the nucleotide sequence is defined by nucleotides 1040-2224 of SEQ ID NO: 1.

12. The isolated nucleotide sequence of claim 7, wherein the nucleotide sequence is defined by nucleotides 1370-2224 of SEQ ID NO: 1.

13. The isolated nucleotide sequence of claim 7, wherein the nucleotide sequence is defined by nucleotides 2084-2224 of SEQ ID NO: 1.

14. The isolated nucleotide sequence of claim 7, wherein the nucleotide sequence is defined by nucleotides 2042-2224 of SEQ ID NO: 1.

15. An isolated nucleotide sequence comprising the nucleic acid sequence defined by nucleotides 1875-1992 of SEQ ID NO: 1, a nucleotide sequence that hybridizes to nucleotides 1875-1992 of SEQ ID NO: 1, or a nucleotide sequence that hybridizes to a compliment of nucleotides 1875-1992 of SEQ ID NO: 1, wherein hybridization condition is 65° C. over night in 7% SDS; 0.5M NaPO₄; 10 mM EDTA, followed by two washes at 50° C. in 0.1×SSC, 0.1% SDS for 30 minutes each, wherein the nucleotide sequence exhibits regulatory element activity.

16. The isolated nucleotide sequence of claim 15, wherein the nucleotide sequence is defined by nucleotides 1875-2084 of SEQ ID NO: 1.

17. The isolated nucleotide sequence of claim 15, wherein the nucleotide sequence is present in tandem.

18. An isolated nucleotide sequence comprising the nucleic acid sequence defined by nucleotides 1-1660 of SEQ ID NO: 1, a nucleotide sequence that hybridizes to nucleotides 1875-1660 of SEQ ID NO: 1, or a nucleotide sequence that hybridizes to a compliment of nucleotides 1-1660 of SEQ ID NO: 1, wherein hybridization condition is 65° C. over night in 7% SDS; 0.5M NaPO₄; 10 mM EDTA, followed by two washes at 50° C. in 0.1×SSC, 0.1% SDS for 30 minutes each, wherein the nucleotide sequence is exhibits regulatory element activity.

19. A chimeric construct comprising the isolated nucleotide sequence of claim 1 operatively linked with a coding region of interest.

20. A method of expressing a coding region of interest within a plant comprising introducing the chimeric construct of claim 19 into a plant and expressing the coding region of interest.

21. A plant comprising the chimeric construct of claim 19.

22. A seed comprising the chimeric construct of claim 19.

23. A plant cell comprising the chimeric construct of claim 19.

24. The plant of claim 21, wherein the plant is selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

25. The seed of claim 22, wherein the plant is selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

26. The plant cell of claim 23, wherein the plant is selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

27. An isolated nucleotide sequence comprising the following nucleic acid sequence
TTATAATTAC AAAATTGATT MTAGTWYYTT TAATTTAATR YTTWTACATT
ATTAATTAAY TTAGHWSTTT YAATTYDTTT TCARAAAYYA TTTTACTATK
KTT(T/-)RT AAAAWMAAAR GGRRAAARTG GYTATTTAAA TACYAAC(M/-)
CTATTTYATT TCAATTWTAR CCTAAAATCA R(M/-)CCC(C/-) ARTTARCCCC
(W/-)(A/-) (T/-)(T/-) (Y/-)(C/-) (A/-)(A/-) (A/-)(T/-)
(T/-)(C/-) AAAYGGBMYA KCCCARTTCC TAAA(A/-)Y RACYCDCYCC
TAACCC(K/-) (C/-)(T/-) (T/-)(W/-) (T/-)(C/-) (C/-)(A/-)
(A/-)(C/-) (C/-)(C/-) RCCCKRTTYC CYCTTTTGAT CCAGGYYGTT
GATCATTTTG ATCAACGVCC ARAATTTCCC CYTTYC(Y/-) (K/-)TTTT
TMATTCCCAA ACACC(S/-) CCYAAMYYTA TCCCRTTTCT CACCAACCGC
CAGATMT(R/-) (W/-)(A/-) (T/-)CCTCT TATCTCTCAA ACTCTCTCGA
ACCTTCCCCT AACCCTAGCA GCCTCTCATC ATCCTCACCT CAAAACCCAC
CGGMMWMCAT GGCYTCTMRA G(S/-)(M/-) (K/-)(Y/-) (G/-)(R/-)
(W/-)(M/-) (M/-)(C/-) (C/-)(K/-) (K/-)(R/-) (T/-)(R/-)
(S/-)(T/-) (C/-)(A/-) (S/-)(Y/-) YCCYYD(T/-) (G/-)(Y/-)
(N/-)(M/-) (T/-)(T/-) (A/-),
a nucleotide sequence that hybridizes to the nucleic acid sequence, or a nucleotide sequence that hybridizes to a compliment of the nucleotide sequence, where R is G or A; Y is T or C; M is A or C; K is G or T; S is G or C; W is A or T; B is G or C or T; D is A or G or T; H is A or C or T; and N is A or C or T or G, and wherein hybridization is selected from the group consisting of:
hybridizing overnight in a solution comprising 7% SDS, 0.5M NaPO4 buffer at pH 7.2, and 10 mM EDTA at 65° C. and washing for one hour at 60° C. in a solution comprising 0.1×SSC and 0.1% SDS;
hybridizing overnight in a solution comprising 7% SDS, 0.5M NaPO4 buffer at pH 7.2, and 10 mM EDTA at 65° C. and washing for one hour at 65° C. in a solution comprising 2×SSC and 0.1% SDS; and
hybridizing overnight in a solution comprising 4×SSC at 65° C. and washing one hour in 0.1×SSC at 65° C., and
wherein the nucleotide sequence exhibits regulatory element activity.

28. A chimeric construct comprising the isolated nucleotide sequence of claim 27 operatively linked with a coding region of interest.

29. A method of expressing a coding region of interest within a plant comprising introducing the chimeric construct of claim 28 into a plant and expressing the coding region of interest.

30. A plant comprising the chimeric construct of claim 28.

31. A seed comprising the chimeric construct of claim 28.

32. A plant cell comprising the chimeric construct of claim 28.

33. The plant of claim 30, wherein the plant is selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

34. The seed of claim 31, wherein the plant is selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.

35. The plant cell of claim 32, wherein the plant is selected from the group consisting of: a monocot plant, a dicot plant, a gymnosperm, an angiosperm, a hardwood tree, a softwood tree, a cereal plant, wheat, barley, oat, corn, tobacco, Brassica, soybean, pea, alfalfa, potato, ginseng, Arabidopsis, a peach, a plum and a spruce.


SEQUENCE LISTING (22 sequences)

SEQ ID NO: 1 - DNA, 2224 bp, Nicotiana tabacum
tctagacttacagaaagtctctaacacgtgagggaatgatccctttccttacctccctgtagagatattggcttttcaacaactagtacataaatatgcgactttgaccgtgtatccccagtcaaaagggaacttcaccctcctagttctttatttccaacatacatggggagtaatgctaaatttacatagaagaataataaaatgaactgtaactaatgatgtactgttccaaagagatgaggacgtcaacatatttattccttcagcccttttcagaataataccataagtagaagaaatggcacataaaatgaagtcctcggcaagtcaaatgtaaatctgaacccacccagctaacccagtgaactcaactttcctggatagatcagcactccttcatgacattgcatgccttctctttaaagagccgcttgatctctgaaaaccaaatgaatctccacagagagatttcgagctccatgagacgccttttggttcttgatttactaaacctataaaaatgaaaggaagtaggacaactgcattttgccgcttaagatgcttcggcgctttgtgaattttaagtcatgagaaagtacaatgttggaatctcacattagaacaatgtatttgtaataacctaggaaagcaaagctagaagggaggtgcagctaaatcttcttctaccttgttatccttgcatttcttgaggaggaggaactgtcctcgcaggtgcaaaatctgcagtcgcccaaaaggatattcagaagtatattacaacatgtttaatggttaaccaagtgaaagatcaaaatagtcattagaacaaaatgcgtgctcagagcgtatctactagttcatcaacccagtacacatctctgaatttcatctcttgccgttgaactaagtcaattggtcaaagacgcataacatgagagacactcataaaacggctgaataacatgcagaagacgtcatgcgccttaggtctcattatgcatgagattattagttatatgctccttcagtttgactagaaatgaaaaatcagttaagcctgtaacgaaatgataacctgcttcaagaagattagactatttttcataaaatatgcagtgccgtgaaatagatacttaatcttaggcaggaaaaatcttctattgggccataataagaactaccaattagaaaggaggtagaaagctccgatactgttatgaaggccattctaagtgctgatgtgaatttcccaatacaaaatgacaacaaaaacaaaagcctcaatcctaagctagttggggtcgctatataaatcctcgacatccatttaactccacttggactcctttctttccaatattttaatattgttagattaatcataaaattgcttagctttctactggcacttaacctactgcaaccctcctcttctgggattccaacacaaacaactaagaggaatttgaaaaaaagaaagcaaatgtgagaagagacaaaatgtacaatgatacctcttcttgcagcaaaggaggcaggttctctgctgagacaaggttctctatttcctgcaagaccttcgtatcttttattcgagaccatgtatgtggaggtaacgccagcaatagtgctgtcagcacatcgttgcttgcaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccaggccgttgatcattttgatcaacgcccagaatttccccttttccttttttaattcccaaacacccctaactctatcccatttctcaccaaccgccacatatgaatcctcttatctctcaaactctctcgaaccttcccctaaccctagcagcctctcatcatcctcacctcaaaacccaccggaatacatggcttctcaagccgtggaaaccttatactcacctccctttgctctta

SEQ ID NO: 2 - DNA, 188 bp, artificial
  Nde-Sma fragment of T1275
catatgaatcctcttatctctcaaactctctcgaaccttcccctaaccctagcagcctctcatcatcctcacctcaaaacccaccggaatacatggcttctcaagccgtggaaaccttatactcacctccctttgctcttacagtactcggccgtcgaccgcggtacccgggtggtcagtcccttatg

SEQ ID NO: 3 - DNA, 129 bp, artificial
  delta N, with Kozak sequence
agatctatcctcttatctctcaaactctctcgaaccttcccctaaccctagcagcctctcatcatcctcacctcaaaacccaccggccaccatggcctctagaggaccccgggtggtcagtcccttatg

SEQ ID NO: 4 - DNA, 119 bp, artificial
  delta N, without Kozak sequence
agatctatcctcttatctctcaaactctctcgaaccttcccctaaccctagcagcctctcatcatcctcacctcaaaacccaccggtctagaggatccccgggtggtcagtcccttatg

SEQ ID NO: 5 - DNA, 602 bp, Nicotiana tabacum
  misc_feature (600)..(600): where “n” is a or g or c or t/u, unknown or other
aattgtaagcgggataacaatttcacacaggaaacagctatgaccatgattacgccaagcttttaatacgactcactatagggaaagcttataattacaaaattgattctagtatttttaatttaatatttttacattattaattaatttagaagttttaattttttttcagaaatcattttactatttttataaaaacaaaagggaaaagtggttatttaaatactagccctatttcatttcaattatagcctaaaatcagccccaattaaccccaattccaaattcaaacgggccagcccaattcctaaaatgacccgctcctaacccgcttttccaacccgcccggtttccccttttgatccaggctgttgatcattttgatcaacggccagaatttcccctttcctttttaattcccaaacaccccccaaccttatcccgtttctcaccaaccgccagatctatcctcttatctctcaaactctctcgaaccttcccctaaccctagcagcctctcatcatcctcacctcaaaacccaccggccaccatggcctctagaggatccccgggtggtcagtcccttatgttacgtcctnaa

SEQ ID NO: 6 - DNA, 610 bp, Nicotiana tabacum
atgttgtgtggaattgtgagcgggataacaatttcacacaggaaacagctatgaccatgattacgccaagctctaatacgactcactatagggaaagcttataattacaaaattgattctagtatttttaatttaatatttatacattattaattaacttagtactttcaattcgttttcaaaaattattttactattttttgtaaaataaaagggagaaaatggctatttaaatactagccctattttatttcaattttagcctaaaatcagcccccaattaaccccaatttcaaattcaaatgggacagcccaattcctaaaataacccgcccctaaccctcttatccaacccacccgatttccccttttgatccaggttgttgatcattttgatcaacgaccagaatttcccccttcctgtttttaattcccaaacaccccccaaccctatcccatttctcaccaaccgccagatctatcctcttatctctcaaactctctcgaaccttcccctaaccctagcagcctctcatcatcctcacctcaaaacccaccggccaccatggcctctagaggatccccgggtggtcagtcccttatgtgcgtc

SEQ ID NO: 7 - DNA, 507 bp, Nicotiana tabacum
  misc_feature (478)..(478): where “n” is a or g or c or t/u, unknown or other
ttgattctagtttttttaatttaatatttatacattattaattaatttagttctttcaatttgttttcagaaattattttactattttttataaaataaaagggagaaaatggctatttaaataccagccctattttatttcaattttaacctaaaatcagccccagttagccccaaacggcccatcccaattcctaaaataactcgcccctaacccgcttatccaacccgcccggttccccttttgatccaggccgttgatcattttgatcaacgaccagaatttcccctttccttttttaattcccaaacaccgccaaacctatcccatttctcaccaaccgccagatctatcctcttatctctcaaactctctcgaaccttcccctaaccctagcagcctctcatcatcctcacctcaaaacccaccggccaccatggcctctagaggatccccgggtggtcagtcccttatgtnacgncctaaatgnccgncctgnnnnnnnc

SEQ ID NO: 8 - DNA, 599 bp, Nicotiana tabacum
  misc_feature (69)..(69): where “n” is a or g or c or t/u, unknown or other
ggaattgtgagcggataacaatttcacacaggaaacagctatgaccatgattacgccaagctctaatangactcactatagggaaagcttataattacaaatttgattctagtatttttaatttaatatttatacattattaattaatttagtactttcaatttgttttcagaaatcattttactatggtttataaaataaaagggagaaaatggctatttaaatactagccctattttatttcaattttagcctaaaatcagccccaattaacccctatttcaaattcaaacgggctagcccagttcctaaaataaccctcccctaacccgcttatccaacccgccctgtttccccttttgatccaggccgttgatcattttgatcaacgaccaaaatttcccctttccttttttaattcccaaacacccccaaccctatcccatttctcaccaaccgccagatctatcctcttatctctcaaactctctcgaaccttcccctaaccctagcagcctctcatcatcctcacctcaaaacccaccggccaccatggcctctagaggatccccgggtggtcagtcccttatgttacgtcc

SEQ ID NO: 9 - DNA, 616 bp, Nicotiana tabacum
  misc_feature (616)..(616): where “n” is a or g or c or t/u, unknown or other
tttatgcttccggctcgtatgttgtgtggaattgtgagcggataacaatttcacacaggaaacagctatgaccatgattacgccaagctctaatacgactcactatagggaaagcttataattacaaaattgattatagtacttttaatttaatatttatacattattaattaatttagcactttcaatttattttcagaaaccattttactattttttataaaataaaagggacaaaatggctatttaaataccaacactattttatttcaattttagcctaaaatcaaacccaattaaccccaaacgggccagcccaattcctaaaacaacccgcccctaacccgcttatccaacccgcccgatttcctcttttgatccaggccgttgatcattttgatcaacggccagaatttcccctttccttttttcattcccaaacacccccaaacctatcccatttctcaccaaccgccagatctatcctcttatctctcaaactctctcgaaccttcccctaaccctagcagcctctcatcatcctcacctcaaaacccaccggccaccatggcctctagaggatccccgggtggtcagtcccttatgttacgtcctn

SEQ ID NO: 10 - DNA, 29 bp, artificial
  primer
ttgcctgcaggggatcttctgcaagcatc

SEQ ID NO: 11 - DNA, 27 bp, artificial
  primer
tcaaatgcatggatcaaaaggggaaac

SEQ ID NO: 12 - DNA, 28 bp, artificial
  primer
ggagctgcaggctatttaaatactagcc

SEQ ID NO: 13 - DNA, 29 bp, artificial
  primer
cactctgcaggcaagacccttcctctata

SEQ ID NO: 14 - DNA, 28 bp, artificial
  primer
atataagctttggggtttctacaggacg

SEQ ID NO: 15 - DNA, 31 bp, artificial
  primer
gagaagatctccaaacacccctaactctatc

SEQ ID NO: 16 - DNA, 24 bp, artificial
  primer
ttgatcattttgatcaacgcccag

SEQ ID NO: 17 - DNA, 31 bp, artificial
  primer
agggggtgcatatgaattaaaaaaggaaaag

SEQ ID NO: 18 - DNA, 51 bp, primer
gcataagagccgccactaaaataagaccgatcaaataagagccgccatgca

SEQ ID NO: 19 - DNA, 30 bp, artificial
  primer
ttaagatttaattaagcttataattacaaa

SEQ ID NO: 20 - DNA, 26 bp, artificial
  primer
attcagatctggcggttggtgagaaa

SEQ ID NO: 21 - DNA, 518 bp, Nicotiana tabacum
  misc_feature (104)..(104): where “n” is t or other (blank)
ttataattacaaaattgattmtagtwyytttaatttaatryttwtacattattaattaayttaghwstttyaattydttttcaraaayyattttactatkkttnrtaaaawmaaarggrraaartggytatttaaatacyaacnctatttyatttcaattwtarcctaaaatcarncccnarttarccccnnnnnnnnnnnnaaayggbmyakcccarttcctaaanyracycdcycctaacccnnnnnnnnnnnnnrccckrttyccycttttgatccaggyygttgatcattttgatcaacgvccaraatttccccyttycnntttttmattcccaaacaccnccyaamyytatcccrtttctcaccaaccgccagatmtnnnncctcttatctctcaaactctctcgaaccttcccctaaccctagcagcctctcatcatcctcacctcaaaacccaccggmmwmcatggcytctmragnnnnnnnnnnnnnnnnnnnnnnyccyydnnnnnnnn

SEQ ID NO: 22 - DNA, 2241 bp, Nicotiana tabacum
  misc_feature (1827)..(1827): where “n” is t or other (blank)
tctagacttacagaaagtctctaacacgtgagggaatgatccctttccttacctccctgtagagatattggcttttcaacaactagtacataaatatgcgactttgaccgtgtatccccagtcaaaagggaacttcaccctcctagttctttatttccaacatacatggggagtaatgctaaatttacatagaagaataataaaatgaactgtaactaatgatgtactgttccaaagagatgaggacgtcaacatatttattccttcagcccttttcagaataataccataagtagaagaaatggcacataaaatgaagtcctcggcaagtcaaatgtaaatctgaacccacccagctaacccagtgaactcaactttcctggatagatcagcactccttcatgacattgcatgccttctctttaaagagccgcttgatctctgaaaaccaaatgaatctccacagagagatttcgagctccatgagacgccttttggttcttgatttactaaacctataaaaatgaaaggaagtaggacaactgcattttgccgcttaagatgcttcggcgctttgtgaattttaagtcatgagaaagtacaatgttggaatctcacattagaacaatgtatttgtaataacctaggaaagcaaagctagaagggaggtgcagctaaatcttcttctaccttgttatccttgcatttcttgaggaggaggaactgtcctcgcaggtgcaaaatctgcagtcgcccaaaaggatattcagaagtatattacaacatgtttaatggttaaccaagtgaaagatcaaaatagtcattagaacaaaatgcgtgctcagagcgtatctactagttcatcaacccagtacacatctctgaatttcatctcttgccgttgaactaagtcaattggtcaaagacgcataacatgagagacactcataaaacggctgaataacatgcagaagacgtcatgcgccttaggtctcattatgcatgagattattagttatatgctccttcagtttgactagaaatgaaaaatcagttaagcctgtaacgaaatgataacctgcttcaagaagattagactatttttcataaaatatgcagtgccgtgaaatagatacttaatcttaggcaggaaaaatcttctattgggccataataagaactaccaattagaaaggaggtagaaagctccgatactgttatgaaggccattctaagtgctgatgtgaatttcccaatacaaaatgacaacaaaaacaaaagcctcaatcctaagctagttggggtcgctatataaatcctcgacatccatttaactccacttggactcctttctttccaatattttaatattgttagattaatcataaaattgcttagctttctactggcacttaacctactgcaaccctcctcttctgggattccaacacaaacaactaagaggaatttgaaaaaaagaaagcaaatgtgagaagagacaaaatgtacaatgatacctcttcttgcagcaaaggaggcaggttctctgctgagacaaggttctctatttcctgcaagaccttcgtatcttttattcgagaccatgtatgtggaggtaacgccagcaatagtgctgtcagcacatcgttgcttgcaggggatcttctgcaagcatctctatttcctgaaggtctaacctcgaagatttaagatttaattacgtttataattacaaaattgattmtagtwyytttaatttaatryttwtacattattaattaayttaghwstttyaattydttttcaraaayyattttactatkkttnrtaaaawmaaarggrraaartggytatttaaatacyaacnctatttyatttcaattwtarcctaaaatcarncccnarttarccccnnnnnnnnnnnnaaayggbmyakcccarttcctaaanyracycdcycctaacccnnnnnnnnnnnnnrccckrttyccycttttgatccaggyygttgatcattttgatcaacgvccaraatttccccyttycnntttttmattcccaaacaccnccyaamyytatcccrtttctcaccaaccgccagatmtnnnncctcttatctctcaaactctctcgaaccttcccctaaccctagcagcctctcatcatcctcacctcaaaacccaccggmmwmcatggcytctmragnnnnnnnnnnnnnnnnnnnnnnyccyydnnnnnnnn